(19) United States
(12) Patent Application Publication
(10) Pub. No.: US 2005/0171132 A1
(43) Pub. Date: Aug. 4, 2005

(54) Diaminopyrimidine derivatives as selective growth hormone secrectgogue receptor (GHS-R) antagonists

(76) Inventors: Zhili Xin, Lake Bluff, IL (US); Bo Liu, Waukegan, IL (US); Gang Liu, Gurnee, IL (US); Mei Liu, Gurnee, IL (US); Michael D. Serby, Mundelein, IL (US); Hongyu Zhao, Buffalo Grove, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/948,042

(22) Filed: Sep. 23, 2004

Related U.S. Application Data
(60) Provisional application No. 60/506,663, filed on Sep. 26, 2003.

(51) Int. Cl. A61K 31/506; C07D 43/02

(52) U.S. Cl. 514/275; 544/234

(57) ABSTRACT

The present invention is related to compounds of formula (I), 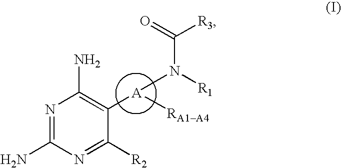 or a therapeutically suitable salt or prodrug thereof, the preparation of the compounds, compositions containing the compounds and the use of the compounds in the prevention or treatment of disorders regulated by the action of ghrelin receptor, including Prader-Willi syndrome, eating disorder, weight gain, weight-loss maintainance following diet and exercise, obesity and disorders associated with obesity such as non-insulin dependent diabetes mellitus.

This application claims priority from U.S. Provisional Patent Application Ser. No. 60/506,663, filed Sep. 26, 2003, which is incorporated herein by reference.

### TECHNICAL FIELD

The present invention is directed to compounds that are selective antagonists of the growth hormone secrectgogue receptor (GHS-R), the preparation of the compounds, compositions containing the compounds and the use of the compounds in the prevention or treatment of disorders regulated by the activation of GHS-R, including Prader-Willi syndrome, eating disorder, weight gain, weight-loss maintainance following diet and exercise, obesity and disorders associated with obesity such as non-insulin dependent diabetes mellitus.

### BACKGROUND OF THE INVENTION

Obesity is a common and very serious public health problem as it increases a person's risk for a number of serious conditions, including diabetes, heart disease, stroke, high blood pressure and some types of cancers. Considerable increase in the number of obese individuals over the past two decades has created profound public health implications. Although studies have demonstrated that reduction in obesity by diet and exercise reduces the associated risk factors dramatically, these treatments are largely unsuccessful considering obesity is strongly associated with genetically inherited factors that contribute to increased appetite, preferences for highly caloric foods, reduced physical activity and increased lipogenic metabolism.

Growth hormone (GH) is not only of importance for linear body growth but is also of major importance for the maintenance of body composition, metabolism and heart function in adult life. GH release from the anterior pituitary is regulated by the stimulatory peptide GH-releasing hormone (GHRH) and the inhibitory peptide somatostatin, Frohman, L., Jansson, J.-O., Endocr. Rev. (1986) 7:223-253. Early research identified small GH-releasing peptides (GHRPs) derived from the pentapeptide met-enkephalin, Momany, F., et. al., Endocrinology (1981) 108:31-39. Further efforts led to the development of a number of peptidyl and non-peptidyl growth hormone secrectgogues (GHSs), including the orally-active, non-peptidyl GH secretagogue MK677, Svensson, J., et. al., J. Clin. Endocrinol. Metab. (1998) 83:362-369. Later efforts cloned a seven-transmembrane G-protein coupled receptor (GPCR) that was a target for the GHSs, Howard, A., et. al., Science (1996) 273:974-977.

This GHS-receptor (GHS-R) is localized in the hypothalamus and in the pituitary, but also in other brain areas such as the hippocampus as well as the pancreas. Recently, an endogenous ligand for the GHS-R, ghrelin, an acylated peptide consisting of 28 amino acids was isolated, Kojima, M., et. al., Nature (1999) 402:656-660. Since then, ghrelin has been found to be localized in the hypothalamic-pituitary area where it stimulates the release of GH to the circulation, but is also found in the highest concentration in the stomach.

Biological evidence indicates that ghrelin has an important role in the regulation of metabolism and energy expenditure. Ghrelin was found to stimulate food intake and weight gain when administered either systemically or intraventricularly in rodents, Nakazato M, et. al., Nature 2001;409:194-198; Asakawa A, et. al., Gastroenterology (2001) 120:337-345. Ghrelin was also found to be more potent than any other orexigenic peptide except neuropeptide Y (NPY). The orexigenic activity of centrally administered ghrelin is thought to be mediated by brain NPY and AGRP, two neuropeptides with potent orexigenic actions, Kamegai, J., et. al., Endocrinology (2000) 141 :4797-4800. It was also recognized that the appetite activity of centrally administered ghrelin can be blocked by co-administration of a NPY-Y1 receptor antagonist. In addition, ghrelin was found to reverse leptin-induced inhibition of food intake, Shintani, M., et. al., Diabetes (2001) 50:227-232. Ghrelin exerts its actions in the arcuate nucleus and paraventricular nucleus to influence the interplay of NPY, AGRP and a-MSH circuits. Ghrelin may also act via afferent vagal pathways that terminate in the hypothalamus. In obese patients, the increase in the plasma ghrelin level with diet-induced weight loss is consistent with the hypothesis that ghrelin has a role in the long-term regulation of body weight. Gastric bypass in obese patients is associated with markedly suppressed ghrelin levels, possibly contributing to the weight-reducing effect of the procedure, Cummings, D. E., et. al., N Engl J Med (2002) 346:1623-30.

Intracerebroventricular treatment with the anti-ghrelin antiserum against the N-terminal region twice a day for 5 days in rats decreased significantly both daily food intake and body weight, Murakami, N., et. al., Journal of Endocrinology (2002) 174, 283-288. Transgenic (Tg) rats expressing an antisense ghrelin receptor mRNA under the control of the promoter for tyrosine hydroxylase (TH) selectively attenuated ghrelin receptor protein expression in the arcuate nucleus (Arc). Tg rats had lower body weight and less adipose tissue than did control rats. Daily food intake was reduced and the stimulatory effect of GHS treatment on feeding was abolished in Tg rats, Shuto, Y., et. al., J. Clin. Invest. (2002) 109:1429-1436. More recently, a peptide-based GHS-R antagonist, [D-Lys-3]-GHRP, was found to decrease energy intake in lean mice, in mice with diet induced obesity and in ob/ob obese mice. It also reduced the rate of gastric emptying. Repeated aministration of this GHS-R antagonist decreased body weight and improved glycemic control in ob/ob mice, Asakawa, A. et. al., Gut, (2003), 52:947-952. These data suggest that GHS-R antagonists may be beneficial in the treatment of Prader-Willi syndrome, eating disorder, weight gain, weight-loss maintainance following diet and exercise, obesity and disorders associated with obesity such as non-insulin dependent diabetes mellitus.

Diaminopyrimidines have been investigated as dihydrofolate reductase (DHFR) inhibitors for the indication of anti-malaria, anti-imflammation and anti-neoplasty. The present invention provides novel diaminopyrimidine-based GHS-R antagonists which antagonize potently the action of ghrelin and do not inhibit the function of DHFR at 10μM concentration or higher, making them suitable for drug development as anti-obesity therapeutical agents with much improved safety profiles.

### BRIEF DESCRIPTION OF THE FIGURES

FIG. 1: In vivo results depicting the effects of Example B on the body weight of mice.

FIG. 2: In vivo results depicting the effects of Example B on fat pat weight in mice.

FIG. 3: In vivo results depicting the effects of Example B on plasma insulin levels in mice.

### SUMMARY OF THE INVENTION

The principle embodiment of the present invention is directed to a compound of formula 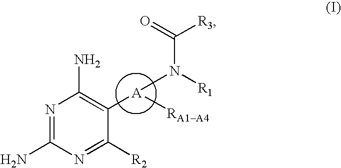
or a therapeutically suitable salt or prodrug thereof, wherein
A is a member selected from the group consisting of aryl, heteroaryl and heterocycle;
R₁ is a member selected from the group consisting of hydrogen, alkoxyalkyl, alkyl, aryl, arylalkyl, cycloalkyl, cycloalkylalkyl, haloalkyl, heteroaryl, heteroarylalkyl, heterocycle and heterocyclealkyl;
R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylsulfinyl, alkylsulfinylalkyl, alkylsulfonyl, alkylsulfonylalkyl, alkylthio, alkylthioalkyl, alkynyl, alkynylalkoxyalkyl, aryl, arylalkenyl, arylalkenyloxyalkyl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, haloalkoxy, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, (R_{C}R_{D}N)carbonylalkenyl, (R_{C}R_{D}N)carbonylalkyl, (R_{C}R_{D}N)sulfonyl and (R_{C}R_{D}N)sulfonylalkyl;
R₃ is a member selected from the group consisting of hydrogen, alkenyl, alkenyloxy, alkoxy, alkoxyalkoxy, alkoxyalkoxyalkyl, alkoxyalkyl, alkyl, alkylcarbonylalkyl, alkylsulfonylalkyl, alkylthioalkyl, alkynyl, aryl, arylalkoxy, arylalkoxyalkyl, arylalkyl, arylcarbonylalkyl, aryloxy, aryloxyalkyl, arylthioalkyl, cyanoalkyl, cycloalkenyl, cycloalkenylalkoxy, cycloalkenylalkoxyalkyl, cycloalkenylalkyl, cycloalkyl, cycloalkylalkoxy, cycloalkylalkoxyalkyl, cycloalkylalkyl, cycloalkylalkylthioalkyl, cycloalkyloxy, cycloalkyloxyalkyl, cycloalkylthioalkyl, haloalkoxy, haloalkyl, heteroaryl, heteroarylalkoxy, heteroarylalkoxyalkyl, heteroarylalkyl, heteroarylalkylthioalkyl, heteroaryloxy, heteroaryloxyalkyl, heteroarylthioalkyl, heterocycle, heterocyclealkoxy, heterocyclealkoxyalkyl, heterocyclealkyl, heterocyclealkylthioalkyl, heterocycleoxy, heterocycleoxyalkyl, heterocyclethioalkyl, hydroxyalkyl, R_{G}R_{H}N— and (R_{G}R_{H}N)alkyl;
R_{C} and R_{D} are each independently a member selected from the group consisting of hydrogen, alkoxyalkyl, alkoxyalkylcarbonyl, alkoxycarbonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (R_{E}R_{F}N)alkyl and (R_{E}R_{F}N)carbonyl, or R_{C} and R_{D} together with the nitrogen atom to which they are attached form a heterocycle;
R_{E} and R_{F} are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl;
R_{G} and R_{H} are each independently a member selected from the group consisting of hydrogen, alkenyl, alkenyloxy, alkoxyalkyl, alkoxyalkylcarbonyl, alkoxycarbonyl, alkoxysulfonyl, alkyl, alkylcarbonyl, alkyloxy, alkylsulfonyl, alkylthioalkyl, alkylthioalkylcarbonyl, alkylthiocarbonyl, aryl, arylalkoxy, arylalkoxyalkyl, arylalkyl, arylcarbonyl, cycloalkyl, cycloalkylalkoxyalkyl, cycloalkylalkyl, cycloalkylcarbonyl, cycloalkylalkoxy, formyl, haloalkyl, heteroaryl, heteroarylalkoxyalkyl, heteroarylalkyl, heteroarylcarbonyl, heteroarylalkoxy, heterocycle, heterocyclealkoxyalkyl, heterocyclealkyl, heterocyclecarbonyl, heterocyclealkoxy, (R_{J}R_{K}N)alkyl and (R_{J}R_{K}N)carbonyl, or R_{G} and R_{H} together with the nitrogen atom to which they are attached form a heterocycle;
R_{J} and R_{K} are each independently a member selected from the group consisting of hydrogen, alkoxycarbonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl;
R_{A1}, R_{A2},R_{A3} and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, formyl, haloalkoxy, haloalkyl, halogen, heteroaryl, heterocycle, hydroxy, hydroxyalkyl, nitro, R_{M}R_{N}N—, (R_{M}R_{N}N)alkyl, (R_{M}R_{N}N)carbonyl and (R_{M}R_{N}N)sulfonyl; and
R_{M} and R_{N} are each independently a member selected from the group consisting of hydrogen, alkoxycarbonyl, alkyl, alkylcarbonyl, alkoxysulfonyl, alkylsulfonyl, aryl, arylalkyl and formyl, or R_{M} and R_{N} together with the nitrogen atom to which they are attached form a heterocycle.

According to another embodiment, the present invention is directed to a pharmaceutical composition comprising a therapeutically effective amount of a compound of formula (I) and a pharmaceutically suitable carrier.

According to another embodiment, the present invention is directed to a method of treating a disorder regulated by GHS-Rs in a mammal, comprising administring of a compound of formula (I).

According to another embodiment, the present invention is directed to a method of treating disorders regulated by the activation of GHS-R, including Prader-Willi syndrome, eating disorder, weight gain, weight-loss maintainance following diet and exercise, obesity and disorders associated with obesity such as non-insulin dependent diabetes mellitus in a mammal comprising administrating a compound of formula (I).

### DETAILED DESCRIPTION OF THE INVENTION

The principle embodiment of the present invention is directed to a compound of formula 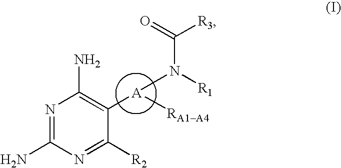
or a therapeutically suitable salt or prodrug thereof, wherein
A is a member selected from the group consisting of aryl, heteroaryl and heterocycle;
R₁ is a member selected from the group consisting of hydrogen, alkoxyalkyl, alkyl, aryl, arylalkyl, cycloalkyl, cycloalkylalkyl, haloalkyl, heteroaryl, heteroarylalkyl, heterocycle and heterocyclealkyl;
R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylsulfinyl, alkylsulfinylalkyl, alkylsulfonyl, alkylsulfonylalkyl, alkylthio, alkylthioalkyl, alkynyl, alkynylalkoxyalkyl, aryl, arylalkenyl, arylalkenyloxyalkyl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, haloalkoxy, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, (R_{C}R_{D}N)carbonylalkenyl, (R_{C}R_{D}N)carbonylalkyl, (R_{C}R_{D}N)sulfonyl and (R_{C}R_{D}N)sulfonylalkyl;
R₃ is a member selected from the group consisting of hydrogen, alkenyl, alkenyloxy, alkoxy, alkoxyalkoxy, alkoxyalkoxyalkyl, alkoxyalkyl, alkyl, alkylcarbonylalkyl, alkylsulfonylalkyl, alkylthioalkyl, alkynyl, aryl, arylalkoxy, arylalkoxyalkyl, arylalkyl, arylcarbonylalkyl, aryloxy, aryloxyalkyl, arylthioalkyl, cyanoalkyl, cycloalkenyl, cycloalkenylalkoxy, cycloalkenylalkoxyalkyl, cycloalkenylalkyl, cycloalkyl, cycloalkylalkoxy, cycloalkylalkoxyalkyl, cycloalkylalkyl, cycloalkylalkylthioalkyl, cycloalkyloxy, cycloalkyloxyalkyl, cycloalkylthioalkyl, haloalkoxy, haloalkyl, heteroaryl, heteroarylalkoxy, heteroarylalkoxyalkyl, heteroarylalkyl, heteroarylalkylthioalkyl, heteroaryloxy, heteroaryloxyalkyl, heteroarylthioalkyl, heterocycle, heterocyclealkoxy, heterocyclealkoxyalkyl, heterocyclealkyl, heterocyclealkylthioalkyl, heterocycleoxy, heterocycleoxyalkyl, heterocyclethioalkyl, hydroxyalkyl, R_{G}R_{H}N— and (R_{G}R_{H}N)alkyl;
R_{C} and R_{D} are each independently a member selected from the group consisting of hydrogen, alkoxyalkyl, alkoxyalkylcarbonyl, alkoxycarbonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (R_{E}R_{F}N)alkyl and (R_{E}R_{F}N)carbonyl, or R_{C} and R_{D} together with the nitrogen atom to which they are attached form a heterocycle;
R_{E} and R_{F} are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl;
R_{G} and R_{H} are each independently a member selected from the group consisting of hydrogen, alkenyl, alkenyloxy, alkoxyalkyl, alkoxyalkylcarbonyl, alkoxycarbonyl, alkoxysulfonyl, alkyl, alkylcarbonyl, alkyloxy, alkylsulfonyl, alkylthioalkyl, alkylthioalkylcarbonyl, alkylthiocarbonyl, aryl, arylalkoxy, arylalkoxyalkyl, arylalkyl, arylcarbonyl, cycloalkyl, cycloalkylalkoxyalkyl, cycloalkylalkyl, cycloalkylcarbonyl, cycloalkylalkoxy, formyl, haloalkyl, heteroaryl, heteroarylalkoxyalkyl, heteroarylalkyl, heteroarylcarbonyl, heteroarylalkoxy, heterocycle, heterocyclealkoxyalkyl, heterocyclealkyl, heterocyclecarbonyl, heterocyclealkoxy, (R_{J}R_{K}N)alkyl and (R_{J}R_{K}N)carbonyl, or R_{G} and R_{H} together with the nitrogen atom to which they are attached form a heterocycle;
R_{J} and R_{K} are each independently a member selected from the group consisting of hydrogen, alkoxycarbonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl;
R_{A1}, R_{A2},R_{A3} and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, formyl, haloalkoxy, haloalkyl, halogen, heteroaryl, heterocycle, hydroxy, hydroxyalkyl, nitro, R_{M}R_{N}N—, (R_{M}R_{N}N)alkyl, (R_{M}R_{N}N)carbonyl and (R_{M}R_{N}N)sulfonyl; and
R_{M} and R_{N} are each independently a member selected from the group consisting of hydrogen, alkoxycarbonyl, alkyl, alkylcarbonyl, alkoxysulfonyl, alkylsulfonyl, aryl, arylalkyl and formyl, or R_{M} and R_{N} together with the nitrogen atom to which they are attached form a heterocycle.

In another embodiment of the present invention there is disclosed a compound of formula (I), wherein A is phenyl; R₁ is a member selected from the group consisting of hydrogen, alkoxyalkyl, alkyl, aryl, arylalkyl, cycloalkyl, cycloalkylalkyl, haloalkyl, heteroaryl, heteroarylalkyl, heterocycle and heterocyclealkyl; R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylsulfinyl, alkylsulfinylalkyl, alkylsulfonyl, alkylsulfonylalkyl, alkylthio, alkylthioalkyl, alkynyl, alkynylalkoxyalkyl, aryl, arylalkenyl, arylalkenyloxyalkyl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, haloalkoxy, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, (R_{C}R_{D}N)carbonylalkenyl, (R_{C}R_{D}N)carbonylalkyl, (R_{C}R_{D}N)sulfonyl and (R_{C}R_{D}N)sulfonylalkyl; R₃ is a member selected from the group consisting of hydrogen, alkenyl, alkenyloxy, alkoxy, alkoxyalkoxy, alkoxyalkoxyalkyl, alkoxyalkyl, alkyl, alkylcarbonylalkyl, alkylsulfonylalkyl, alkylthioalkyl, alkynyl, aryl, arylalkoxy, arylalkoxyalkyl, arylalkyl, arylcarbonylalkyl, aryloxy, aryloxyalkyl, arylthioalkyl, cyanoalkyl, cycloalkenyl, cycloalkenylalkoxy, cycloalkenylalkoxyalkyl, cycloalkenylalkyl, cycloalkyl, cycloalkylalkoxy, cycloalkylalkoxyalkyl, cycloalkylalkyl, cycloalkylalkylthioalkyl, cycloalkyloxy, cycloalkyloxyalkyl, cycloalkylthioalkyl, haloalkoxy, haloalkyl, heteroaryl, heteroarylalkoxy, heteroarylalkoxyalkyl, heteroarylalkyl, heteroarylalkylthioalkyl, heteroaryloxy, heteroaryloxyalkyl, heteroarylthioalkyl, heterocycle, heterocyclealkoxy, heterocyclealkoxyalkyl, heterocyclealkyl, heterocyclealkylthioalkyl, heterocycleoxy, heterocycleoxyalkyl, heterocyclethioalkyl, hydroxyalkyl, R_{G}R_{H}N— and (R_{G}R_{H}N)alkyl; —R_{C} and R_{D} are each independently a member selected from the group consisting of hydrogen, alkoxyalkyl, alkoxyalkylcarbonyl, alkoxycarbonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (R_{E}R_{F}N)alkyl and (R_{E}R_{F}N)carbonyl, or R_{C} and R_{D} together with the nitrogen atom to which they are attached form a heterocycle; R_{E} and R_{F} are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl; R_{G} and R_{H} are each independently a member selected from the group consisting of hydrogen, alkenyl, alkenyloxy, alkoxyalkyl, alkoxyalkylcarbonyl, alkoxycarbonyl, alkoxysulfonyl, alkyl, alkylcarbonyl, alkyloxy, alkylsulfonyl, alkylthioalkyl, alkylthioalkylcarbonyl, alkylthiocarbonyl, aryl, arylalkoxy, arylalkoxyalkyl, arylalkyl, arylcarbonyl, cycloalkyl, cycloalkylalkoxyalkyl, cycloalkylalkyl, cycloalkylcarbonyl, cycloalkylalkoxy, formyl, haloalkyl, heteroaryl, heteroarylalkoxyalkyl, heteroarylalkyl, heteroarylcarbonyl, heteroarylalkoxy, heterocycle, heterocyclealkoxyalkyl, heterocyclealkyl, heterocyclecarbonyl, heterocyclealkoxy, (R_{J}R_{K}N)alkyl and (R_{J}R_{K}N)carbonyl, or R_{G} and R_{H} together with the nitrogen atom to which they are attached form a heterocycle; R_{J} and R_{K} are each independently a member selected from the group consisting of hydrogen, alkoxycarbonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl; and R_{A1}, R_{A2}, R_{A3} and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, formyl, haloalkoxy, haloalkyl, halogen, heteroaryl, heterocycle,hydroxy, hydroxyalkyl, nitro, R_{M}R_{N}N—, (R_{M}R_{N}N)alkyl, (R_{M}R_{N}N)carbonyl and (R_{M}R_{N}N)sulfonyl; and R_{M} and R_{N} are each independently a member selected from the group consisting of hydrogen, alkoxycarbonyl, alkyl, alkylcarbonyl, alkoxysulfonyl, alkylsulfonyl, aryl, arylalkyl and formyl, or R_{M} and R_{N} together with the nitrogen atom to which they are attached form a heterocycle.

In another embodiment of the present invention there is disclosed a compound of formula (I), wherein A is phenyl, R₁ is a member selected from the group consisting of hydrogen, alkoxyalkyl, alkyl, arylalkyl, cycloalkylalkyl, haloalkyl, heteroarylalkyl, heterocyclealkyl; R₂ is a member selected from the group consisting of alkenyl, alkenyloxyalkyl, alkoxyalkyl, alkoxycarbonyl, alkyl, alkylcarbonyl, aryl, arylalkoxyalkyl, arylalkyl, aryloxyalkyl, carboxy, carboxyalkyl, haloalkoxy, heteroaryl, heteroarylalkoxyalkyl, heteroarylalkyl, heteroaryloxyalkyl, heterocycle, heterocyclealkoxyalkyl, heterocyclealkyl, heterocycleoxyalkyl, hydroxyalkyl, R_{C}R_{D}N—, R_{C}R_{D}N)alkyl and (R_{C}R_{D}N)carbonylalkyl; R₃ is a member selected from the group consisting of hydrogen, alkenyl, alkenyloxy, alkoxy, alkoxyalkoxy, alkoxyalkoxyalkyl, alkoxyalkyl, alkyl, alkylcarbonylalkyl, alkylsulfonylalkyl, alkylthioalkyl, alkynyl, aryl, arylalkoxy, arylalkoxyalkyl, arylalkyl, arylcarbonylalkyl, aryloxy, aryloxyalkyl, arylthioalkyl, cyanoalkyl, cycloalkenyl, cycloalkenylalkoxy, cycloalkenylalkoxyalkyl, cycloalkenylalkyl, cycloalkyl, cycloalkylalkoxy, cycloalkylalkoxyalkyl, cycloalkylalkyl, cycloalkylalkylthioalkyl, cycloalkyloxy, cycloalkyloxyalkyl, cycloalkylthioalkyl, haloalkoxy, haloalkyl, heteroaryl, heteroarylalkoxy, heteroarylalkoxyalkyl, heteroarylalkyl, heteroarylalkylthioalkyl, heteroaryloxy, heteroaryloxyalkyl, heteroarylthioalkyl, heterocycle, heterocyclealkoxy, heterocyclealkoxyalkyl, heterocyclealkyl, heterocyclealkylthioalkyl, heterocycleoxy, heterocycleoxyalkyl, heterocyclethioalkyl, hydroxyalkyl, R_{G}R_{H}N— and (R_{G}R_{H}N)alkyl; R_{C} and R_{D} are each independently a member selected from the group consisting of hydrogen, alkoxyalkyl, alkoxyalkylcarbonyl, alkoxycarbonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (R_{E}R_{F}N)alkyl and (R_{E}R_{F}N)carbonyl, or R_{C} and R_{D} together with the nitrogen atom to which they are attached form a heterocycle; R_{E} and R_{F} are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl; R_{G} and R_{H} are each independently a member selected from the group consisting of hydrogen, alkenyl, alkenyloxy, alkoxyalkyl, alkoxyalkylcarbonyl, alkoxycarbonyl, alkoxysulfonyl, alkyl, alkylcarbonyl, alkyloxy, alkylsulfonyl, alkylthioalkyl, alkylthioalkylcarbonyl, alkylthiocarbonyl, aryl, arylalkoxy, arylalkoxyalkyl, arylalkyl, arylcarbonyl, cycloalkyl, cycloalkylalkoxyalkyl, cycloalkylalkyl, cycloalkylcarbonyl, cycloalkylalkoxy, formyl, haloalkyl, heteroaryl, heteroarylalkoxyalkyl, heteroarylalkyl, heteroarylcarbonyl, heteroarylalkoxy, heterocycle, heterocyclealkoxyalkyl, heterocyclealkyl, heterocyclecarbonyl, heterocyclealkoxy, (R_{J}R_{K}N)alkyl and (R_{J}R_{K}N)carbonyl, or R_{G} and R_{H} together with the nitrogen atom to which they are attached form a heterocycle; R_{J} and R_{K} are each independently a member selected from the group consisting of hydrogen, alkoxycarbonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl; and R_{A1}, R_{A2}, R_{A3} and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, formyl, haloalkoxy, haloalkyl, halogen, heteroaryl, heterocycle, hydroxy, hydroxyalkyl, nitro, R_{M}R_{N}N—, (R_{M}R_{N}N)alkyl, (R_{M}R_{N}N)carbonyl and (R_{M}R_{N}N)sulfonyl; and R_{M} and R_{N} are each independently a member selected from the group consisting of hydrogen, alkoxycarbonyl, alkyl, alkylcarbonyl, alkoxysulfonyl, alkylsulfonyl, aryl, arylalkyl and formyl, or R_{M} and R_{N} together with the nitrogen atom to which they are attached form a heterocycle.

In another embodiment of the present invention there is disclosed a compound of formula (I), wherein A is phenyl, R₁ is a member selected from the group consisting of hydrogen, alkoxyalkyl, alkyl, arylalkyl, cycloalkylalkyl, haloalkyl, heteroarylalkyl, heterocyclealkyl;
R₂ is a member selected from the group consisting of alkenyl, alkenyloxyalkyl, alkoxyalkyl, alkoxycarbonyl, alkyl, alkylcarbonyl, aryl, arylalkoxyalkyl, arylalkyl, aryloxyalkyl, carboxy, carboxyalkyl, haloalkoxy, heteroaryl, heteroarylalkoxyalkyl, heteroarylalkyl, heteroaryloxyalkyl, heterocycle, heterocyclealkoxyalkyl, heterocyclealkyl, heterocycleoxyalkyl, hydroxyalkyl, - R_{C}R_{D}N, (R_{C}R_{D}N)alkyl and (R_{C}R_{D}N)carbonylalkyl; and
R₃ is a member selected from the group consisting of hydrogen, alkenyl, alkenyloxy, alkoxy, alkoxyalkoxyalkyl, alkoxyalkyl, alkyl, alkylcarbonylalkyl, aryl, arylalkoxyalkyl, arylalkyl, arylcarbonylalkyl, aryloxyalkyl, cycloalkenyl, cycloalkenylalkoxyalkyl, cycloalkyl, cycloalkylalkoxyalkyl, cycloalkylalkyl, cycloalkyloxyalkyl, haloalkyl, heteroaryl, heteroarylalkoxyalkyl, heteroarylalkyl, heteroaryloxyalkyl, heterocycle, heterocyclealkoxyalkyl, heterocyclealkyl, heterocycleoxyalkyl, hydroxy, hydroxyalkyl, nitro, R_{G}R_{H}N— and (R_{G}R_{H}N)alkyl; R_{C} and R_{D} are each independently a member selected from the group consisting of hydrogen, alkoxyalkyl, alkoxyalkylcarbonyl, alkoxycarbonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (R_{E}R_{F}N)alkyl and (R_{E}R_{F}N)carbonyl, or R_{C} and R_{D} together with the nitrogen atom to which they are attached form a heterocycle; R_{E} and R_{F} are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl; R_{G} and R_{H} are each independently a member selected from the group consisting of hydrogen, alkenyl, alkenyloxy, alkoxyalkyl, alkoxyalkylcarbonyl, alkoxycarbonyl, alkoxysulfonyl, alkyl, alkylcarbonyl, alkyloxy, alkylsulfonyl, alkylthioalkyl, alkylthioalkylcarbonyl, alkylthiocarbonyl, aryl, arylalkoxy, arylalkoxyalkyl, arylalkyl, arylcarbonyl, cycloalkyl, cycloalkylalkoxyalkyl, cycloalkylalkyl, cycloalkylcarbonyl, cycloalkylalkoxy, formyl, haloalkyl, heteroaryl, heteroarylalkoxyalkyl, heteroarylalkyl, heteroarylcarbonyl, heteroarylalkoxy, heterocycle, heterocyclealkoxyalkyl, heterocyclealkyl, heterocyclecarbonyl, heterocyclealkoxy, (R_{J}R_{K}N)alkyl and (R_{J}R_{K}N)carbonyl, or R_{G} and R_{H} together with the nitrogen atom to which they are attached form a heterocycle; R_{J} and R_{K} are each independently a member selected from the group consisting of hydrogen, alkoxycarbonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl; and R_{A1}, R_{A2}, R_{A3} and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, formyl, haloalkoxy, haloalkyl, halogen, heteroaryl, heterocycle,hydroxy, hydroxyalkyl, nitro, R_{M}R_{N}N—, (R_{M}R_{N}N)alkyl, (R_{M}R_{N}N)carbonyl and (R_{M}R_{N}N)sulfonyl; and R_{M} and R_{N} are each independently a member selected from the group consisting of hydrogen, alkoxycarbonyl, alkyl, alkylcarbonyl, alkoxysulfonyl, alkylsulfonyl, aryl, arylalkyl and formyl, or R_{M} and R_{N} together with the nitrogen atom to which they are attached form a heterocycle.

In another embodiment of the present invention there is disclosed a compound of formula (I), wherein A is phenyl, R₁ is a member selected from the group consisting of hydrogen, alkoxyalkyl, alkyl, arylalkyl and haloalkyl; R₂ is a member selected from the group consisting of alkenyloxyalkyl, alkoxyalkyl, alkoxycarbonyl, alkyl, alkylcarbonyl, arylalkoxyalkyl, arylalkyl, aryloxyalkyl, carboxy, carboxyalkyl, haloalkoxy, heteroarylalkoxyalkyl, heteroarylalkyl, heteroaryloxyalkyl, heterocyclealkoxyalkyl, heterocyclealkyl, heterocycleoxyalkyl, hydroxyalkyl, R_{C}R_{D}N—, (R_{C}R_{D}N)alkyl and (R_{C}R_{D}N)carbonylalkyl; R₃ is a member selected from the group consisting of hydrogen, alkenyl, alkenyloxy, alkoxy, alkoxyalkoxyalkyl, alkoxyalkyl, alkyl, alkylcarbonylalkyl, aryl, arylalkoxyalkyl, arylalkyl, arylcarbonylalkyl, aryloxyalkyl, cycloalkenyl, cycloalkenylalkoxyalkyl, cycloalkyl, cycloalkylalkoxyalkyl, cycloalkylalkyl, cycloalkyloxyalkyl, formyl, haloalkyl, heteroaryl, heteroarylalkoxyalkyl, heteroarylalkyl, heteroaryloxyalkyl, heterocycle, heterocyclealkoxyalkyl, heterocyclealkyl, heterocycleoxyalkyl, hydroxy, hydroxyalkyl, nitro, R_{G}R_{H}N— and (R_{G}R_{H}N)alkyl; R_{C} and R_{D} are each independently a member selected from the group consisting of hydrogen, alkoxyalkyl, alkoxyalkylcarbonyl, alkoxycarbonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (R_{E}R_{F}N)alkyl and (R_{E}R_{F}N)carbonyl, or R_{C} and R_{D} together with the nitrogen atom to which they are attached form a heterocycle; R_{E} and R_{F} are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl; R_{G} and R_{H} are each independently a member selected from the group consisting of hydrogen, alkenyl, alkenyloxy, alkoxyalkyl, alkoxyalkylcarbonyl, alkoxycarbonyl, alkoxysulfonyl, alkyl, alkylcarbonyl, alkyloxy, alkylsulfonyl, alkylthioalkyl, alkylthioalkylcarbonyl, alkylthiocarbonyl, aryl, arylalkoxy, arylalkoxyalkyl, arylalkyl, arylcarbonyl, cycloalkyl, cycloalkylalkoxyalkyl, cycloalkylalkyl, cycloalkylcarbonyl, cycloalkylalkoxy, formyl, haloalkyl, heteroaryl, heteroarylalkoxyalkyl, heteroarylalkyl, heteroarylcarbonyl, heteroarylalkoxy, heterocycle, heterocyclealkoxyalkyl, heterocyclealkyl, heterocyclecarbonyl, heterocyclealkoxy, (R_{J}R_{K}N)alkyl and (R_{J}R_{K}N)carbonyl, or R_{G} and R_{H} together with the nitrogen atom to which they are attached form a heterocycle; R_{J} and R_{K} are each independently a member selected from the group consisting of hydrogen, alkoxycarbonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl; and R_{A1}, R_{A2}, R_{A3} and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, formyl, haloalkoxy, haloalkyl, halogen, heteroaryl, heterocycle,hydroxy, hydroxyalkyl, nitro, R_{M}R_{N}N—, (R_{M}R_{N}N)alkyl, (R_{M}R_{N}N)carbonyl and (R_{M}R_{N}N)sulfonyl; and R_{M} and R_{N} are each independently a member selected from the group consisting of hydrogen, alkoxycarbonyl, alkyl, alkylcarbonyl, alkoxysulfonyl, alkylsulfonyl, aryl, arylalkyl and formyl, or R_{M} and R_{N} together with the nitrogen atom to which they are attached form a heterocycle.

The compound of formula (I), wherein A is phenyl; R₁ is a member selected from the group consisting of hydrogen and arylalkyl; R₂ is a member selected from the group consisting of alkenyloxyalkyl, alkoxyalkyl, alkyl, arylalkoxyalkyl and heteroarylalkoxyalkyl; R₃ is a member selected from the group consisting of hydrogen, alkenyloxy, alkoxy, alkoxyalkyl, alkyl, aryl, arylalkoxyalkyl, arylalkyl, arylcarbonylalkyl, cycloalkenyl, cycloalkyl, cycloalkylalkyl, formyl, heteroarylalkyl, heterocycle and R_{G}R_{H}N—; and R_{G} and R_{H} are each independently a member selected from the group consisting of hydrogen, alkyl, alkylthioalkyl, aryl, arylalkyl, cycloalkyl, cycloalkylalkyl and haloalkyl.

According to another embodiment, the present invention is directed to a pharmaceutical composition comprising a therapeutically effective amount of a compound of formula (I) and a pharmaceutically suitable carrier.

According to another embodiment, the present invention is directed to a method of treating a disorder regulated by GHS-Rs in a mammal, comprising administring of a compound of formula (I).

According to another embodiment, the present invention is directed to a method of treating disorders regulated by the activation of GHS-R, including Prader-Willi syndrome, eating disorder, weight gain, weight-loss maintainance following diet and exercise, obesity and disorders associated with obesity such as non-insulin dependent diabetes mellitus in a mammal comprising administrating a compound of formula (I).

### DEFINITIONS

As used throughout this specification and the appended claims, the following terms have the following meanings:

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl and 3-decenyl.

The term “alkenyloxy” as used herein, means an alkenyl group, as defined herein, appended to the parent molecular moiety through an oxygen atom.

The term “alkenyloxyalkyl” as used herein, means an alkenyloxy group, as defined herein, appended to the parent molecular moiety through an alkyl group, as defined herein.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, n-butoxy, tert-butoxy, pentyloxy and hexyloxy.

The term “alkoxyalkoxy” as used herein, means an alkoxy group, as defined herein, appended to the parent molecular moiety through an alkoxy group. Representative example of alkoxyalkoxy include, but are not limited to, 2-(methoxy)ethoxy, 2-(ethoxy)ethoxy, 3-(methoxy)propoxy and 2-(n-butoxy)ethoxy.

The term “alkoxyalkoxyalkyl” as used herein, means an alkoxyalkoxy group, as defined herein, appended to the parent molecular moiety through an alkyl group, as defined herein. Representative examples of alkoxyalkoxyalkyl include, but are not limited to, 2-(methoxy)ethoxymethyl, 2-(ethoxy)ethoxymethyl, 3-(methoxy)propoxymethyl, 2-(n-butoxy)ethoxymethyl and 2-(tert-butoxy)ethoxymethyl.

The term “alkoxyalkyl” as used herein, means an alkoxy group, as defined herein, appended to the parent molecular moiety through an alkyl group, as defined herein. Representative examples of alkoxyalkyl include, but are not limited to, n-butoxymethyl, tert-butoxymethyl, 2-(ethoxy)ethyl, 2-methoxyethyl and methoxymethyl.

The term “alkoxyalkylcarbonyl” as used herein, means an alkoxyalkyl group, as defined herein, appended to the parent molecular moiety through a carbonyl group, as defined herein. Representative examples of alkoxyalkylcarbonyl include, but are not limited to, n-butoxymethylcarbonyl, tert-butoxymethylcarbonyl, 2-(ethoxy)ethylcarbonyl, 2-methoxyethylcarbonyl and methoxymethylcarbonyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl and tert-butoxycarbonyl.

The term “alkoxycarbonylalkyl” as used herein, means an alkoxycarbonyl group, as defined herein, appended to the parent molecular moiety through a alkyl group, as defined herein.

The term “alkoxysulfonyl” as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl and tert-butoxysulfonyl.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl and 1-oxopentyl.

The term “alkylcarbonylalkyl” as used herein, means an alkylcarbonyl group, as defined herein, appended to the parent molecular moiety through an alkyl group, as defined herein. Representative examples of alkylcarbonylalkyl include, but are not limited to, 2-oxopropyl, 3-oxobutyl, 3-oxopentyl and 4-oxopentyl.

The term “alkylcarbonyloxy” as used herein, means an alkylcarbonyl group, as defined herein, appended to the parent molecular moiety through an oxygen atom, as defined herein. Representative examples of alkylcarbonyloxy include, but are not limited to, acetyloxy, propionyloxy, 3-oxobutyl and butyryloxy.

The term “alkylsulfinyl” as used herein, means an alkyl group, as defined herein, appended to the parent molecular moiety through a sulfinyl group, as defined herein. Representative examples of alkylsulfinyl include, but are not limited to, methylsulfinyl and ethylsulfinyl.

The term “alkylsulfinylalkyl” as used herein, means an alkylsulfinyl group, as defined herein, appended to the parent molecular moiety through an alkyl group, as defined herein. Representative examples of alkylsulfinylalkyl include, but are not limited to, methylsulfinylmethyl and ethylsulfinylmethyl.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylsulfonylalkyl” as used herein, means an alkylsulfonyl group, as defined herein, appended to the parent molecular moiety through an alkyl group, as defined herein. Representative examples of alkylsulfonyl include, but are not limited to, methylsulfonylmethyl and ethylsulfonylmethyl.

The term “alkylthio” as used herein, means an alkyl group, as defined herein, appended to the parent molecular moiety through a sulfur atom. Representative examples of alkylthio include, but are not limited to, methylthio and ethylthio.

The term “alkylthioalkyl” as used herein, means an alkylthio group, as defined herein, appended to the parent molecular moiety through an alkyl group, as defined herein. Representative examples of alkylthioalkyl include, but are not limited to, methylthiomethyl and ethylthiomethyl.

The term “alkylthioalkylcarbonyl” as used herein, means an alkylthioalkyl group, as defined herein, appended to the parent molecular moiety through a carbonyl group, as defined herein. Representative examples of alkylthioalkylcarbonyl include, but are not limited to, methylthiomethylcarbonyl and ethylthiomethylcarbonyl.

The term “alkylthiocarbonyl” as used herein, means an alkylthio group, as defined herein, appended to the parent molecular moiety through a carbonyl group, as defined herein. Representative examples of alkylthiocarbonyl include, but are not limited to, methylthiocarbonyl and ethylthiocarbonyl.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl and 1-butynyl.

The term “alkynyloxy” as used herein, means an alkynyl group, as defined herein, appended to the parent molecular moiety through an oxygen atom. Representative examples of alkynyloxy include, but are not limited, to but-3-ynyloxy and hex-4-ynyloxy.

The term “alkynyloxyalkyl” as used herein, means an alkynyloxy group, as defined herein, appended to the parent molecular moiety through an alkyl group, as defined herein. Representative examples of alkynyloxyalkyl include, but are not limited, to but-3-ynyloxymethyl and hex-4-ynyloxymethyl.

The term, “alkynylalkoxy” as used herein, means an alkynyl group, as defined herein, appended to the parent molecular moiety through an alkoxy group, as defined herein.

The term, “alkynylalkoxyalkyl” as used herein, refers to an alkynylalkoxy group, as defined herein, appended to the parent molecular moiety through an alkyl group, as defined herein.

The term “aryl” as used herein, means a phenyl group, or a bicyclic or a tricyclic fused ring system wherein one or more of the fused rings is a phenyl group. Bicyclic fused ring systems are exemplified by a phenyl group appended to the parent molecular moiety, which is fused to a cycloalkyl group, as defined herein, a phenyl group, a heteroaryl, as defined herein, or a heterocycle as defined herein. Tricyclic fused ring systems are exemplified by an aryl bicyclic fused ring system fused to a cycloalkyl group, as defined herein, a phenyl group, a heteroaryl, as defined herein, or a heterocycle as defined herein. Representative examples of aryl include, but are not limited to, anthracenyl, azulenyl, fluorenyl, indanyl, indenyl, naphthyl, phenyl and tetrahydronaphthyl.

The aryl groups of this invention can be substituted with 0, 1, 2, 3, 4, or 5 substituents independently a member selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, aryl, arylcarbonyl, arylsulfonyl, carboxy, carboxyalkyl, cyano, cyanoalkyl, formyl, haloalkoxy, haloalkyl, haloalkylcarbonyl, haloalkylsulfonyl, halogen, heteroaryl, heterocycle, hydroxy, hydroxyalkyl, hydroxyhaloalkyl, mercapto, nitro, Z₅Z₆N— and (Z₅Z₆N)alkyl, wherein the substituent aryl, the aryl of arylcarbonyl, the aryl of arylsulfonyl, the heteroaryl and the heterocycle can be substituted with 0, 1, or 2 substitutents independently selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, haloalkoxy, haloalkyl, haloalkylcarbonyl, haloalkylsulfonyl, halogen, hydroxy and hydroxyalkyl. Representative examples include, but are not limited to, 2-bromophenyl, 2-chlorophenyl, 3-chlorophenyl, 4-chlorophenyl, 3-cyanophenyl, 4-cyanophenyl, 2,3-dichlorophenyl, 3,4-dichlorophenyl, 2,5-dichlorophenyl, 2,4-dimethylphenyl, 3,5-dimethylphenyl, 2-fluoro-3-methylphenyl, 2-fluorophenyl, 3-fluorophenyl, 4-fluorophenyl, 2-methoxyphenyl, 3-methoxyphenyl, 4-methoxyphenyl, 2-methylphenyl, 3-methylphenyl, 4-(methylthio)phenyl, 4-nitrophenyl, 4-(trifluoromethoxy)phenyl and 3-(trifluoromethyl)phenyl.

The term “arylalkenyl” as used herein, means an aryl group, as defined herein, appended to the parent molecular moiety through an alkenyl group, as defined herein.

The term “arylalkenyloxy” as used herein, means an arylalkenyl group, as defined herein, appended to the parent molecular moiety through an oxy group, as defined herein.

The term “arylalkenyloxyalkyl” as used herein, means an arylalkenyoxy group, as defined herein, appended to the parent molecular moiety through an alkyl group, as defined herein.

The term “arylalkoxy” as used herein, means an aryl group, as defined herein, appended to the parent molecular moiety through an alkoxy group, as defined herein. Representative examples of arylalkoxy include, but are not limited to, benzyloxy, 2-bromobenzyloxy, 2-chlorobenzyloxy, 3-chlorobenzyloxy, 4-chlorobenzyloxy, 2-(4-chlorophenyl)ethoxy, 3-cyanobenzyloxy, 4-cyanobenzyloxy, 2,3-dichlorobenzyloxy, 2,5-dichlorobenzyloxy, 2,4-dimethylbenzyloxy, 3,5-dimethylbenzyloxy, 2-fluoro-3-methylbenzyloxy, 2-fluorobenzyloxy, 4-fluorobenzyloxy, 2-methoxybenzyloxy, 3-methoxybenzyloxy, 4-methoxybenzyloxy, 2-methylbenzyloxy, 3-methylbenzyloxy, 4-(methylthio)benzyloxy, 4-nitrobenzyloxy, 4-(trifluoromethoxy)benzyloxy and 3-(trifluoromethyl)benzyloxy.

The term “arylalkoxyalkyl” as used herein, means an arylalkoxy group, as defined herein, appended to the parent molecular moiety through an alkyl group, as defined herein. Representative examples of arylalkoxyalkyl include, but are not limited to, benzyloxymethyl, 2-bromobenzyloxymethyl, 2-chlorobenzyloxymethyl, 3-chlorobenzyloxymethyl, 4-chlorobenzyloxymethyl, 4-cyanobenzyloxymethyl, 2,3-dichlorobenzyloxymethyl, 2,5-dichlorobenzyloxymethyl, 2,4-dimethylbenzyloxymethyl, 3,5-dimethylbenzyloxymethyl, 2-fluoro-3-methylbenzyloxymethyl, 2-fluorobenzyloxymethyl, 4-fluorobenzyloxymethyl, 2-methoxybenzyloxymethyl, 3-methoxybenzyloxymethyl, 4-methoxybenzyloxymethyl, 2-methylbenzyloxymethyl, 3-methylbenzyloxymethyl, 4-(methylthio)benzyloxymethyl, 4-nitrobenzyloxymethyl, 4-(trifluoromethoxy)benzyloxymethyl and 3-(trifluoromethyl)benzyloxymethyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 1-phenylethyl, 3-phenylpropyl, 4-phenylbutyl, 2-naphth-2-ylethyl, 2-bromobenzyl, 4-cyanobenzyl, 1-(4-cyanophenyl)ethyl, 2-chlorobenzyl, 3-chlorobenzyl, 4-chlorobenzyl, 1-(4-chlorophenyl)ethyl, 2-(4-chlorophenyl)ethyl, 2,3-dichlorobenzyl, 2,5-dichlorobenzyl, 2,4-dimethylbenzyl, 3,5-dimethylbenzyl, 2-fluoro-3-methylbenzyl, 2-fluorobenzyl, 4-fluorobenzyl, 2-methoxybenzyl, 3-methoxybenzyl, 4-methoxybenzyl, 2-methylbenzyl, 3-methylbenzyl, 4-(methylthio)benzyl, 4-nitrobenzyl, 1-(4-nitrophenyl)ethyl, 2-(4-chlorophenyl)ethyl, 4-(trifluoromethoxy)benzyl and 3-(trifluoromethyl)benzyl.

The term “arylalkylthio” as used herein, means an arylalkyl group, as defined herein, appended to the parent molecular moiety through a sulfur atom. Representative examples of arylalkylthio include, but are not limited to, benzylthio, 2-phenylethylthio, 1-phenylethylthio, 3-phenylpropylthio, 4-phenylbutylthio, 2-naphth-2-ylethylthio, 2-bromobenzylthio, 4-cyanobenzylthio, 1-(4-cyanophenyl)ethyl, 2-chlorobenzylthio, 3-chlorobenzylthio, 4-chlorobenzylthio, 1-(4-chlorophenyl)ethylthio, 2-(4-chlorophenyl)ethylthio, 2,3-dichlorobenzylthio, 2,5-dichlorobenzylthio, 2,4-dimethylbenzylthio, 3,5-dimethylbenzylthio, 2-fluoro-3-methylbenzylthio, 2-fluorobenzylthio, 4-fluorobenzylthio, 2-methoxybenzylthio, 3-methoxybenzylthio, 4-methoxybenzylthio, 2-methylbenzylthio, 3-methylbenzylthio, 4-(methylthio)benzylthio, 4-nitrobenzylthio, 1-(4-nitrophenyl)ethylthio, 2-(4-chlorophenyl)ethylthio, 4-(trifluoromethoxy)benzylthio and 3-(trifluoromethyl)benzylthio.

The term “arylalkylthioalkyl” as used herein, means an arylalkylthio group, as defined herein, appended to the parent molecular moiety through an alkyl group, as defined herein. Representative examples of arylalkylthio include, but are not limited to, benzylthiomethyl, 2-phenylethylthiomethyl, 1-phenylethylthiomethyl, 3-phenylpropylthiomethyl, 4-phenylbutylthiomethyl, 2-naphth-2-ylethylthiomethyl, 2-bromobenzylthiomethyl, 4-cyanobenzylthiomethyl, 1-(4-cyanophenyl)ethylmethyl, 2-chlorobenzylthiomethyl, 3-chlorobenzylthiomethyl, 4-chlorobenzylthiomethyl, 1-(4-chlorophenyl)ethylthiomethyl, 2-(4-chlorophenyl)ethylthiomethyl, 2,3-dichlorobenzylthiomethyl, 2,5-dichlorobenzylthiomethyl, 2,4-dimethylbenzylthiomethyl, 3,5-dimethylbenzylthiomethyl, 2-fluoro-3-methylbenzylthiomethyl, 2-fluorobenzylthiomethyl, 4-fluorobenzylthiomethyl, 2-methoxybenzylthiomethyl, 3-methoxybenzylthiomethyl, 4-methoxybenzylthiomethyl, 2-methylbenzylthiomethyl, 3-methylbenzylthiomethyl, 4-(methylthio)benzylthiomethyl, 4-nitrobenzylthiomethyl, 1-(4-nitrophenyl)ethylthiomethyl, 2-(4-chlorophenyl)ethylthiomethyl, 4-(trifluoromethoxy)benzylthiomethyl and 3-(trifluoromethyl)benzylthiomethyl.

The term “arylcarbonyl” as used herein, means an aryl group, as defined herein, appended to the parent molecular moiety through a carbonyl group, as defined herein. Representative examples of arylcarbonyl include, but are not limited to, benzoyl, naphthoyl, 2-bromo benzoyl, 2-chlorobenzoyl, 3-chlorobenzoyl, 4-chlorobenzoyl, 3-cyanobenzoyl, 4-cyanobenzoyl, 2,3-dichlorobenzoyl, 3,4-dichlorobenzoyl, 2,5-dichlorobenzoyl, 2,4-dimethylbenzoyl, 3,5-dimethylbenzoyl, 2-fluoro-3-methylbenzoyl, 2-fluorobenzoyl, 3-fluorobenzoyl, 4-fluorobenzoyl, 2-methoxybenzoyl, 3-methoxybenzoyl, 4-methoxybenzoyl, 2-methylbenzoyl, 3-methylbenzoyl, 4-(methylthio)benzoyl, 4-nitrobenzoyl, 4-(trifluoromethoxy)benzoyl and 3-(trifluoromethyl)benzoyl.

The term “arylcarbonylalkyl” as used herein, means an arylcarbonyl group, as defined herein, appended to the parent molecular moiety through an alkyl group, as defined herein.

The term “aryloxy” as used herein, means an aryl group, as defined herein, appended to the parent molecular moiety through an oxygen atom. Representative examples of aryloxy include, but are not limited to, 2-bromophenoxy, 2-chlorophenoxy, 3-chlorophenoxy, 4-chlorophenoxy, 4-cyanophenoxy, 2,3-dichlorophenoxy, 3,4-dichlorophenoxy, 2,5-dichlorophenoxy, 2,4-dimethylphenoxy, 3,5-dimethylphenoxy, 2-fluoro-3-methylphenoxy, 2-fluorophenoxy, 3-fluorophenoxy, 4-fluorophenoxy, 2-methoxyphenoxy, 3-methoxyphenoxy, 4-methoxyphenoxy, 2-methylphenoxy, 3-methylphenoxy, 4-(methylthio)phenoxy, 3-nitrophenoxy, 4-nitrophenoxy, 4-(trifluoromethoxy)phenoxy and 3-(trifluoromethyl)phenoxy.

The term “aryloxyalkyl” as used herein, means an aryloxy group, as defined herein, appended to the parent molecular moiety through an alkyl group, as defined herein. Representative examples of aryloxyalkyl include, but are not limited to, 2-(2-bromophenoxy)ethyl, 2-(2-chlorophenoxy)ethyl, 3-chlorophenoxymethyl, 4-chlorophenoxymethyl, 4-cyanophenoxymethyl, 2,3-dichlorophenoxymethyl, 3,4-dichlorophenoxymethyl, 2,5-dichlorophenoxymethyl, 2,4-dimethylphenoxymethyl, 3,5-dimethylphenoxymethyl, 2-fluoro-3-methylphenoxymethyl, 2-fluorophenoxymethyl, 3-fluorophenoxymethyl, 4-fluorophenoxymethyl, 2-methoxyphenoxymethyl, 3-methoxyphenoxymethyl, 4-methoxyphenoxymethyl, 2-methylphenoxymethyl, 3-methylphenoxymethyl, 4-(methylthio)phenoxymethyl, 3-nitrophenoxymethyl, 4 nitrophenoxymethyl, 4-(trifluoromethoxy)phenoxymethyl and 3-(trifluoromethyl)phenoxymethyl.

The term “arylsulfonyl” as used herein, means an aryl group, as defined herein, appended to the parent molecular moiety through a sulfonyl group, as defined herein. Representative examples of arylsulfonyl include, but are not limited to, phenylsulfonyl, naphthylsulfonyl, 2-bromophenylsulfonyl, 2-chlorophenylsulfonyl, 3-chlorophenylsulfonyl, 4-chlorophenylsulfonyl, 3-cyanophenylsulfonyl, 4-cyanophenylsulfonyl, 2,3-dichlorophenylsulfonyl, 3,4-dichlorophenylsulfonyl, 2,5-dichlorophenylsulfonyl, 2,4-dimethylphenylsulfonyl, 3,5-dimethylphenylsulfonyl, 2-fluoro-3-methylphenylsulfonyl, 2-fluorophenylsulfonyl, 3-fluorophenylsulfonyl, 4-fluorophenylsulfonyl, 2-methoxyphenylsulfonyl, 3-methoxyphenylsulfonyl, 4-methoxyphenylsulfonyl, 2-methylphenylsulfonyl, 3-methylphenylsulfonyl, 4-(methylthio)phenylsulfonyl, 4-nitrophenylsulfonyl, 4-(trifluoromethoxy)phenylsulfonyl and 3-(trifluoromethyl)phenylsulfonyl.

The term “arylthio” as used herein, means an aryl group, as defined herein, appended to the parent molecular moiety through a sulfur atom. Representative examples of arylthio include, but are not limited to, 2-bromophenylthio, 2-chlorophenylthio, 3-chlorophenylthio, 4-chlorophenylthio, 4-cyanophenylthio, 2,3-dichlorophenylthio, 3,4-dichlorophenylthio, 2,5-dichlorophenylthio, 2,4-dimethylphenylthio, 3,5-dimethylphenylthio, 2-fluoro-3-methylphenylthio, 2-fluorophenylthio, 3-fluorophenylthio, 4-fluorophenylthio, 2-methoxyphenylthio, 3-methoxyphenylthio, 4-methoxyphenylthio, 2-methylphenylthio, 3-methylphenylthio, 4-(methylthio)phenylthio, 3-nitrophenylthio, 4-nitrophenylthio, 4-(trifluoromethoxy)phenylthio and 3-(trifluoromethyl)phenylthio.

The term “arylthioalkyl” as used herein, means an arylthio group, as defined herein, appended to the parent molecular moiety through an alkyl group, as defined herein. Representative examples of arylthioalkyl include, but are not limited to, 2-bromophenylthiomethyl, 2-chlorophenylthiomethyl, 3-chlorophenylthiomethyl, 4-chlorophenylthiomethyl, 4-cyanophenylthiomethyl, 2,3-dichlorophenylthiomethyl, 3,4-dichlorophenylthiomethyl, 2,5-dichlorophenylthiomethyl, 2,4-dimethylphenylthiomethyl, 3,5-dimethylphenylthiomethyl, 2-fluoro-3-methylphenylthiomethyl, 2-fluorophenylthiomethyl, 3-fluorophenylthiomethyl, 4-fluorophenylthiomethyl, 2-methoxyphenylthiomethyl, 3-methoxyphenylthiomethyl, 4-methoxyphenylthiomethyl, 2-methylphenylthiomethyl, 3-methylphenylthiomethyl, 4-(methylthio)phenylthiomethyl, 3-nitrophenylthiomethyl, 4-nitrophenylthiomethyl, 4-(trifluoromethoxy)phenylthiomethyl and 3-(trifluoromethyl)phenylthiomethyl.

The term “carbonyl” as used herein, means a —C(═O)— group.

The term “carboxy” as used herein, means a —CO₂H group.

The term “carboxyalkyl” as used herein, means a carboxy group, as defined herein, appended to the parent molecular moiety through an alkyl group, as defined herein. Representative examples of carboxyalkyl include, but are not limited to, carboxymethyl, 2-carboxyethyl and 3-carboxypropyl.

The term “cyano” as used herein, means a —CN group.

The term “cyanoalkyl” as used herein, means a cyano group, as defined herein, appended to the parent molecular moiety through an alkyl group, as defined herein. Representative examples of cyanoalkyl include, but are not limited to, cyanomethyl, 2-cyanoethyl and 3-cyanopropyl.

The term “cycloalkenyl” as used herein, means a cycloalkyl group, as defined herein, which contains 1 or 2 double bonds. The term cycloalkenyl of the present invention may also exist as a bicyclic fused ring system. Bicyclic fused cycloalkenyl ring systems are exemplified by a cycloalkenyl group, as defined herein, appended to the parent molscular moiety, which is fused to a phenyl group. Representative examples of cycloalkenyl include, but are not limited to, cyclopropenyl, cyclobutenyl, cyclopentenyl, cyclohexenyl, cycloheptenyl and cyclooctenyl.

The cycloalkenyl groups of this invention can be substituted with 0, 1, 2, 3, or 4 substituents independently a member selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, halogen, haloalkyl, hydroxy, hydroxyalkyl, mercapto, Z₅Z₆N— and (Z₅Z₆N)alkyl.

The term “cycloalkenylalkoxy” as used herein, means a cycloalkenyl group, as defined herein, appended to the parent molecular moiety through an alkoxy group, as defined herein. Representative examples of cycloalkenylalkoxy include, but are not limited to, cyclopropenylmethoxy, cyclobutenylmethoxy, cyclopentenylmethoxy, cyclohexenylmethoxy, cycloheptenylmethoxy and cyclooctenylmethoxy.

The term “cycloalkenylalkoxyalkyl” as used herein, means a cycloalkenylalkoxy group, as defined herein, appended to the parent molecular moiety through an alkyl group, as defined herein. Representative examples of cycloalkenylalkoxyalkyl include, but are not limited to, cyclopropenylmethoxymethyl, cyclobutenylmethoxymethyl, cyclopentenylmethoxymethyl, cyclohexenylmethoxymethyl, cycloheptenylmethoxymethyl and cyclooctenylmethoxymethyl.

The term “cycloalkenylalkyl” as used herein, means a cycloalkenyl group, as defined herein, appended to the parent molecular moiety through an alkyl group, as defined herein. Representative examples of cycloalkenylalkyl include, but are not limited to, cyclopropenylmethyl, cyclobutenylmethyl, cyclopentenylmethyl, cyclohexenylmethyl, cycloheptenylmethyl and cyclooctenylmethyl.

The term “cycloalkenylalkylthio” as used herein, means a cycloalkenylalkyl group, as defined herein, appended to the parent molecular moiety through a sulfur atom. Representative examples of cycloalkenylalkylthio include, but are not limited to, cyclopropenylmethylthio, cyclobutenylmethylthio, cyclopentenylmethylthio, cyclohexenylmethylthio, cycloheptenylmethylthio and cyclooctenylmethylthio.

The term “cycloalkenylalkylthioalkyl” as used herein, means a cycloalkenylalkylthio group, as defined herein, appended to the parent molecular moiety through an alkyl group, as defined herein. Representative examples of cycloalkenylalkylthioalkyl include, but are not limited to, cyclopropenylmethylthiomethyl, cyclobutenylmethylthiomethyl, cyclopentenylmethylthiomethyl, cyclohexenylmethylthiomethyl, cycloheptenylmethylthiomethyl and cyclooctenylmethylthiomethyl.

The term “cycloalkenyloxy” as used herein, means a cycloalkenyl group, as defined herein, appended to the parent molecular moiety through an oxygen atom. Representative examples of cycloalkenyloxy include, but are not limited to, cyclopropenyloxy, cyclobutenyloxy, cyclopentenyloxy, cyclohexenyloxy, cycloheptenyloxy and cyclooctenyloxy.

The term “cycloalkenyloxyalkyl” as used herein, means a cycloalkenyloxy group, as defined herein, appended to the parent molecular moiety through an alkyl group, as defined herein. Representative examples of cycloalkenyloxyalkyl include, but are not limited to, cyclopropenyloxymethyl, cyclobutenyloxymethyl, cyclopentenyloxymethyl, cyclohexenyloxymethyl, cycloheptenyloxymethyl and cyclooctenyloxymethyl.

The term “cycloalkenylthio” as used herein, means a cycloalkenyl group, as defined herein, appended to the parent molecular moiety through a sulfur atom. Representative examples of cycloalkenylthio include, but are not limited to, cyclopropenylthio, cyclobutenylthio, cyclopentenylthio, cyclohexenylthio, cycloheptenylthio and cyclooctenylthio.

The term “cycloalkenylthioalkyl” as used herein, means a cycloalkenylthio group, as defined herein, appended to the parent molecular moiety through an alkyl group, as defined herein. Representative examples of cycloalkenylthioalkyl include, but are not limited to, cyclopropenylthiomethyl, cyclobutenylthiomethyl, cyclopentenylthiomethyl, cyclohexenylthiomethyl, cycloheptenylthiomethyl and cyclooctenylthiomethyl.

The term “cycloalkyl,” as used herein, refers to a monocyclic, bicyclic, or tricyclic ring system. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic fused ring systems are exemplified by a cycloalkyl group appended to the parent molecular moiety, which is fused to an additional cycloalkyl group, as defined herein, a phenyl group, a heteroaryl, as defined herein, or a heterocycle as defined herein. Tricyclic fused ring systems are exemplified by a cycloalkyl bicyclic fused ring system fused to an additional cycloalkyl group, as defined herein, a phenyl group, a heteroaryl, as defined herein, or a heterocycle as defined herein. The additional fused cycloalkyl group may be substituted but may not be fused to another ring. Bicyclic ring systems are exemplified by a bridged monocyclic ring system in which two non-adjacent carbon atoms of the monocyclic ring are linked by an alkylene bridge of between one and three additional carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two nonadjacent carbon atoms of the bicyclic ring are linked by a bond or an alkylene bridge of between one and three carbon atoms. Representative examples of tricyclic-ring systems include, but are not limited to, tricyclo[3.3.1.0^{3,7}]nonane and tricyclo[3.3.1.1^{3,7}]decane (adamantane).

The cycloalkyl groups of this invention can be substituted with 0, 1, 2, 3, or 4 substituents independently a member selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, halogen, haloalkyl, hydroxy, hydroxyalkyl, mercapto, oxo, Z₅Z₆N and (Z₅Z₆N)alkyl.

The term “cycloalkylalkoxy” as used herein, means a cycloalkyl group, as defined herein, appended to the parent molecular moiety through an alkoxy group, as defined herein. Representative examples of cycloalkylalkoxy include, but are not limited to, cyclopropylmethoxy, cyclobutylmethoxy, cyclopentylmethoxy, cyclohexylmethoxy, 2-cyclohexylethoxy, cycloheptylmethoxy and cyclooctylmethoxy.

The term “cycloalkylalkoxyalkyl” as used herein, means a cycloalkylalkoxy group, as defined herein appended to the parent molecular moiety through an alkyl group, as defined herein. Representative examples of cycloalkoxyalkyl include, but are not limited to, cyclopropylmethoxymethyl, cyclobutylmethoxymethyl, cyclopentylmethoxymethyl, cyclohexylmethoxymethyl, (2-cyclohexylethoxy)methyl, cycloheptylmethoxymethyl and cyclooctylmethoxymethyl.

The term “cycloalkylalkyl” as used herein, means a cycloalkyl group, as defined herein, appended to the parent molecular moiety through an alkyl group as defined herein. Representative examples of cycloalkylalkyl include, but are not limited to, cyclopropylmethyl, cyclobutylmethyl, cyclopentylmethyl, cyclohexylmethyl, 2-cyclohexylethyl, cycloheptylmethyl and cyclooctylmethyl.

The term “cycloalkylalkylthio” as used herein, means a cycloalkylalkyl group, as defined herein, appended to the parent molecular moiety through a sulfur atom. Representative examples of cycloalkylalkylthio include, but are not limited to, cyclopropylmethylthio, cyclobutylmethylthio, cyclopentylmethylthio, cyclohexylmethylthio, 2-cyclohexylethylthio, cycloheptylmethylthio and cyclooctylmethylthio.

The term “cycloalkylalkylthioalkyl” as used herein, means a cycloalkylalkylthio group, as defined herein, appended to the parent molecular moiety through an alkyl group, as defined herein. Representative examples of cycloalkylalkylthioalkyl include, but are not limited to, cyclopropylmethylthiomethyl, cyclobutylmethylthiomethyl, cyclopentylmethylthiomethyl, cyclohexylmethylthiomethyl, 2-cyclohexylethylthiomethyl, cycloheptylmethylthiomethyl and cyclooctylmethylthiomethyl.

The term “cycloalkylcarbonyl” as used herein, means a cycloalkyl group, as defined herein, appended to the parent molecular moiety through a carbonyl group as defined herein. Representative examples of cycloalkylcarbonyl include, but are not limited to, cyclopropylcarbonyl, cyclobutylcarbonyl, cyclopentylcarbonyl, cyclohexylcarbonyl, cycloheptylcarbonyl and cyclooctylcarbonyl.

The term “cycloalkyloxy” as used herein, means a cycloalkyl group, as defined herein, appended to the parent molecular moiety through an oxygen atom, examples of cycloalkyloxy include cyclopropyloxy, cyclobutyloxy, cyclopentyloxy, cyclohexyloxy, cycloheptyloxy and cyclooctyloxy.

The term “cycloalkyloxyalkyl” as used herein, means a cycloalkyloxy group, as defined herein, appended to the parent molecular moiety through an alkyl group, as defined herein. Representative examples of cycloalkyloxyalkyl include, but are not limited to, cyclopropyloxymethyl, cyclobutyloxymethyl, cyclopentyloxymethyl, cyclohexyloxymethyl, cycloheptyloxymethyl and cyclooctyloxymethyl.

The term “cycloalkylthio” as used herein, means a cycloalkyl group, as defined herein, appended to the parent molecular moiety through a sulfur atom, examples of cycloalkylthio include cyclopropylthio, cyclobutylthio, cyclopentylthio, cyclohexylthio, cycloheptylthio and cyclooctylthio.

The term “cycloalkylthioalkyl” as used herein, means a cycloalkylthio group, as defined herein, appended to the parent molecular moiety through an alkyl group, as defined herein. Representative examples of cycloalkylthioalkyl include cyclopropylthiomethyl, cyclobutylthiomethyl, cyclopentylthiomethyl, cyclohexylthiomethyl, cycloheptylthiomethyl and cyclooctylthiomethyl.

The term “formyl,” as used herein, means a —C(O)H group.

The term “formylalkyl” as used herein, means a formyl group, as defined herein, appended to the parent molecular moiety through an alkyl group as defined herein. Representative examples of formylalkyl include, but are not limited to, cyclopropylmethyl, cyclobutylmethyl, cyclopentylmethyl, cyclohexylmethyl, 2-oxoethyl, 3-oxopropyl and 4-oxobutyl.

The term “halo” or “halogen,” as used herein, means —Cl, —Br, —I or —F.

The term “haloalkoxy,” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, pentafluoroethoxy and 2-chloro-3-fluoropentoxy.

The term “haloalkyl,” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl and 2-chloro-3-fluoropentyl.

The term “haloalkylcarbonyl,” as used herein, means a haloalkyl group, as defined herein, appended to the parent molecular moiety through a carbonyl group, as defined herein. Representative examples of haloalkylcarbonyl include, but are not limited to, chloromethylcarbonyl, 2-fluoroethylcarbonyl, trifluoromethylcarbonyl, pentafluoroethylcarbonyl and 2-chloro-3-fluoropentylcarbonyl.

The term “haloalkylsulfonyl,” as used herein, means a haloalkyl group, as defined herein, appended to the parent molecular moiety through a sulfonyl group, as defined herein. Representative examples of haloalkylsulfonyl include, but are not limited to, chloromethylsulfonyl, 2-fluoroethylsulfonyl, trifluoromethylsulfonyl, pentafluoroethylsulfonyl and 2-chloro-3-fluoropentylsulfonyl.

The term “heteroaryl,” as used herein, means an aromatic monocyclic ring or an aromatic bicyclic ring. The aromatic monocyclic rings are five or six membered rings wherein 1, 2, 3, or 4 atoms are independently a member selected from the group consisting of N, O and S. The five membered aromatic monocyclic rings have two double bonds and the six membered aromatic monocyclic rings have three double bonds. The heteroaryl bicyclic rings are exemplified by a heteroaryl monocyclic ring appended to the parent molecular moiety, which is fused to a phenyl group, or another heteroaryl group as herein defined. The heteroaryl monocyclic rings and the heteroaryl bicyclic rings are connected to the parent molecular moiety through a carbon or nitrogen atom. Representative examples of heteroaryl include, but are not limited to, benzimidazole, benzothienyl, benzoxadiazolyl, cinnolinyl, dibenzofuranyl, furopyridinyl, furyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, quinolinyl, tetrazolyl, thiadiazolyl, thiazolyl, thienopyridinyl, thienyl, triazolyl and triazinyl.

The heteroaryl groups of the present invention are substituted with 0, 1, 2, 3, or 4 substituents independently a member selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, aryl, arylcarbonyl, arylsulfonyl, carboxy, carboxyalkyl, cyano, cyanoalkyl, formyl, haloalkoxy, haloalkyl, haloalkylcarbonyl, haloalkylsulfonyl, halogen, heteroaryl, heterocycle, hydroxy, hydroxyalkyl, hydroxyhaloalkyl, mercapto, nitro, Z₅Z₆N— and (Z₅Z₆N)alkyl, wherein the substituent aryl, the aryl of arylcarbonyl, the aryl of arylsulfonyl, the substituent heteroaryl and the substituent heterocycle can be substituted with 0, 1, or 2 substitutents independently selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, haloalkoxy, haloalkyl, haloalkylcarbonyl, haloalkylsulfonyl, halogen, hydroxy and hydroxyalkyl.

The term “heteroarylalkoxy” as used herein, means a heteroaryl group, as defined herein, appended to the parent molecular moiety through an alkoxy group, as defined herein. Representative examples of heteroarylalkoxy include, but are not limited to, fur-3-ylmethoxy, 1H-imidazol-2-ylmethoxy, 1H-imidazol-4-ylmethoxy, 1-(pyridin-4-yl)ethoxy, pyridin-3-ylmethoxy, 6-chloropyridin-3-ylmethoxy, pyridin-4-ylmethoxy, (6-(trifluoromethyl)pyridin-3-yl)methoxy, (6-(cyano)pyridin-3-yl)methoxy, (2-(cyano)pyridin-4-yl)methoxy, (5-(cyano)pyridin-2-yl)methoxy, (2-(chloro)pyridin-4-yl)methoxy, pyrimidin-5-ylmethoxy, 2-(pyrimidin-2-yl)propoxy, thien-2-ylmethoxy and thien-3-ylmethoxy.

The term “heteroarylalkoxyalkyl” as used herein, means a heteroarylalkoxy group, as defined herein, appended to the parent molecular moiety through an alkyl group, as defined herein. Representative examples of heteroarylalkoxyalkyl include, but are not limited to, fur-3-ylmethoxymethyl, 1H-imidazol-2-ylmethoxymethyl, 1H-imidazol-4-ylmethoxymethyl, pyridin-3-ylmethoxymethyl, 6-chloropyridin-3-ylmethoxymethyl, pyridin-4-ylmethoxymethyl, (6-(trifluoromethyl)pyridin-3-yl)methoxymethyl, (6-(cyano)pyridin-3-yl)methoxymethyl, (2-(cyano)pyridin-4-yl)methoxymethyl, (5-(cyano)pyridin-2-yl)methoxymethyl, (2-(chloro)pyridin-4-yl)methoxymethyl, pyrimidin-5-ylmethoxymethyl, 2-(pyrimidin-2-yl)propoxymethyl, thien-2-ylmethoxymethyl and thien-3-ylmethoxymethyl.

The term “heteroarylalkyl” as used herein, means a heteroaryl, as defined herein, appended to the parent molecular moiety through an alkyl group, as defined herein. Representative examples of heteroarylalkyl include, but are not limited to, fur-3-ylmethyl, 1H-imidazol-2-ylmethyl, 1H-imidazol-4-ylmethyl, 1-(pyridin-4-yl)ethyl, pyridin-3-ylmethyl, 6-chloropyridin-3-ylmethyl, pyridin-4-ylmethyl, (6-(trifluoromethyl)pyridin-3-yl)methyl, (6-(cyano)pyridin-3-yl)methyl, (2-(cyano)pyridin-4-yl)methyl, (5-(cyano)pyridin-2-yl)methyl, (2-(chloro)pyridin-4-yl)methyl, pyrimidin-5-ylmethyl, 2-(pyrimidin-2-yl)propyl, thien-2-ylmethyl and thien-3-ylmethyl.

The term “heteroarylalkylthio” as used herein, means a heteroarylalkyl group, as defined herein, appended to the parent molecular moiety through a sulfur atom. Representative examples of heteroarylalkylthio include, but are not limited to, fur-3-ylmethylthio, 1H-imidazol-2-ylmethylthio, 1H-imidazol-4-ylmethylthio, pyridin-3-ylmethylthio, 6-chloropyridin-3-ylmethylthio, pyridin-4-ylmethylthio, (6-(trifluoromethyl)pyridin-3-yl)methylthio, (6-(cyano)pyridin-3-yl)methylthio, (2-(cyano)pyridin-4-yl)methylthio, (5-(cyano)pyridin-2-yl)methylthio, (2-(chloro)pyridin-4-yl)methylthio, pyrimidin-5-ylmethylthio, 2-(pyrimidin-2-yl)propylthio, thien-2-ylmethylthio and thien-3-ylmethylthio.

The term “heteroarylalkylthioalkyl” as used herein, means a heteroarylalkylthio group, as defined herein, appended to the parent molecular moiety through an alkyl group, as defined herein. Representative examples of heteroarylalkylthioalkyl include, but are not limited to, fur-3-ylmethylthiomethyl, 1H-imidazol-2-ylmethylthiomethyl, 1H-imidazol-4-ylmethylthiomethyl, pyridin-3-ylmethylthiomethyl, 6-chloropyridin-3-ylmethylthiomethyl, pyridin-4-ylmethylthiomethyl, (6-(trifluoromethyl)pyridin-3-yl)methylthiomethyl, (6-(cyano)pyridin-3-yl)methylthiomethyl, (2-(cyano)pyridin-4-yl)methylthiomethyl, (5-(cyano)pyridin-2-yl)methylthiomethyl, (2-(chloro)pyridin-4-yl)methylthiomethyl, pyrimidin-5-ylmethylthiomethyl, 2-(pyrimidin-2-yl)propylthiomethyl, thien-2-ylmethylthiomethyl and thien-3-ylmethylthiomethyl.

The term “heteroarylcarbonyl” as used herein, means a heteroaryl group, as defined herein, appended to the parent molecular moiety through a carbonyl group, as defined herein. Representative examples of heteroarylcarbonyl include, but are not limited to, fur-3-ylcarbonyl, 1H-imidazol-2-ylcarbonyl, 1H-imidazol-4-ylcarbonyl, pyridin-3-ylcarbonyl, 6-chloropyridin-3-ylcarbonyl, pyridin-4-ylcarbonyl, (6-(trifluoromethyl)pyridin-3-yl)carbonyl, (6-(cyano)pyridin-3-yl)carbonyl, (2-(cyano)pyridin-4-yl)carbonyl, (5-(cyano)pyridin-2-yl)carbonyl, (2-(chloro)pyridin-4-yl)carbonyl, pyrimidin-5-ylcarbonyl, pyrimidin-2-ylcarbonyl, thien-2-ylcarbonyl and thien-3-ylcarbonyl.

The term “heteroaryloxy” as used herein, means a heteroaryl group, as defined herein, appended to the parent molecular moiety through an oxygen atom. Representative examples of heteroaryloxy include, but are not limited to, fur-3-yloxy, 1H-imidazol-2-yloxy, 1H-imidazol-4-yloxy, pyridin-3-yloxy, 6-chloropyridin-3-yloxy, pyridin-4-yloxy, (6-(trifluoromethyl)pyridin-3-yl)oxy, (6-(cyano)pyridin-3-yl)oxy, (2-(cyano)pyridin-4-yl)oxy, (5-(cyano)pyridin-2-yl)oxy, (2-(chloro)pyridin-4-yl)oxy, pyrimidin-5-yloxy, pyrimidin-2-yloxy, thien-2-yloxy and thien-3-yloxy.

The term “heteroaryloxyalkyl” as used herein, means a heteroaryloxy group, as defined herein, appended to the parent molecular moiety through an alkyl group, as defined herein. Representative examples of heteroaryloxyalkyl include, but are not limited to, fur-3-yloxymethyl, 1H-imidazol-2-yloxymethyl, 1H-imidazol-4-yloxymethyl, pyridin-3-yloxymethyl, 6-chloropyridin-3-yloxymethyl, pyridin-4-yloxymethyl, (6-(trifluoromethyl)pyridin-3-yl)oxymethyl, (6-(cyano)pyridin-3-yl)oxymethyl, (2-(cyano)pyridin-4-yl)oxymethyl, (5-(cyano)pyridin-2-yl)oxymethyl, (2-(chloro)pyridin-4-yl)oxymethyl, pyrimidin-5-yloxymethyl, pyrimidin-2-yloxymethyl, thien-2-yloxymethyl and thien-3-yloxymethyl.

The term “heteroarylthio” as used herein, means a heteroaryl group, as defined herein, appended to the parent molecular moiety through a sulfur atom. Representative examples of heteroarylthio include, but are not limited to, fur-3-ylthio, 1H-imidazol-2-ylthio, 1H-imidazol-4-ylthio, pyridin-3-ylthio, 6-chloropyridin-3-ylthio, pyridin-4-ylthio, (6-(trifluoromethyl)pyridin-3-yl)thio, (6-(cyano)pyridin-3-yl)thio, (2-(cyano)pyridin-4-yl)thio, (5-(cyano)pyridin-2-yl)thio, (2-(chloro)pyridin-4-yl)thio, pyrimidin-5-ylthio, pyrimidin-2-ylthio, thien-2-ylthio and thien-3-ylthio.

The term “heteroarylthioalkyl” as used herein, means a heteroarylthio group, as defined herein, appended to the parent molecular moiety through an alkyl group, as defined herein. Representative examples of heteroarylthioalkyl include, but are not limited to, fir-3-ylthiomethyl, 1H-imidazol-2-ylthiomethyl, 1H-imidazol-4-ylthiomethyl, pyridin-3-ylthiomethyl, 6-chloropyridin-3-ylthiomethyl, pyridin-4-ylthiomethyl, (6-(trifluoromethyl)pyridin-3-yl)thiomethyl, (6-(cyano)pyridin-3-yl)thiomethyl, (2-(cyano)pyridin-4-yl)thiomethyl, (5-(cyano)pyridin-2-yl)thiomethyl, (2-(chloro)pyridin-4-yl)thiomethyl, pyrimidin-5-ylthiomethyl, pyrimidin-2-ylthiomethyl, thien-2-ylthiomethyl and thien-3-ylthiomethyl.

The term “heterocycle,” as used herein, means a non-aromatic monocyclic ring or a non-aromatic bicyclic ring. The non-aromatic monocyclic ring is a three, four, five, six, seven, or eight membered ring containing 1 or 2 heteroatoms independently a member selected from the group consisting of N, O and S. The three membered rings have zero double bonds. The four and five membered rings have zero or one double bond. The six membered rings have zero, one, or two double bonds. The seven and eight membered rings have zero, one, two, or three double bonds. The bicyclic heterocycle rings are composed of a non-aromatic heterocyclic monocyclic ring appended to the parent molecular moiety, which is fused to a cycloalkyl group, as defined herein, or a phenyl group. The heterocycle groups of the present invention can be attached to the parent molecular moiety through a carbon atom or a nitrogen atom. Representative examples of heterocycle include, but are not limited to, azetidinyl, 1,3-benzodioxolyl, 1,3-benzodioxol-4-yl, hexahydro-1H-azepinyl, hexahydroazocin-(2H)-yl, morpholinyl, piperazinyl, piperidinyl, pyrrolidinyl, tetrahydrofuranyl, tetrahydrofuran-2-yl, tetrahydrofuran-3-yl, tetrahydro-2H-pyranyl, tetrahydro-2H-pyran-2-yl, tetrahydro-2H-pyran-4-yl, tetrahydrothienyl, tetrahydrothien-2-yl and tetrahydrothien-3-yl and thiomorpholinyl.

The heterocycles of the present invention are substituted with 0, 1, 2, 3, or 4 substituents independently a member selected from alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, aryl, arylcarbonyl, arylsulfonyl, carboxy, carboxyalkyl, cyano, cyanoalkyl, formyl, haloalkoxy, haloalkyl, halogen, heteroaryl, heterocycle, hydroxy, hydroxyalkyl, mercapto, nitro, oxo, Z₅Z₆N— and (Z₅Z₆N)alkyl, wherein the substituent aryl, the aryl of arylcarbonyl, the aryl of arylsulfonyl, the substituent heteroaryl and the substituent heterocycle can be substituted with 0, 1, or 2 substitutents independently selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, haloalkoxy, haloalkyl, haloalkylcarbonyl, haloalkylsulfonyl, halogen, hydroxy and hydroxyalkyl.

The term “heterocyclealkoxy” as used herein, means a heterocycle group, as defined herein, appended to the parent molecular moiety through an alkoxy group, as defined herein. Representative examples of heterocyclealkoxy include, but are not limited to, 1,3-benzodioxol-4-ylmethoxy, pyridin-3-ylmethoxy, 2-pyrimidin-2-ylpropoxy, tetrahydrofuran-2-ylmethoxy, tetrahydrofuran-3-ylmethoxy, tetrahydro-2H-pyran-2-ylmethoxy, tetrahydro-2H-pyran-4-ylmethoxy, tetrahydrothien-2-ylmethoxy and tetrahydrothien-3-ylmethoxy.

The term “heterocyclealkoxyalkyl” as used herein, means a heterocyclealkoxy group, as defined herein, appended to the parent molecular moiety through an alkyl group, as defined herein. Representative examples of heterocyclealkoxyalkyl include, but are not limited to, 1,3-benzodioxol-4-ylmethoxymethyl, pyridin-3-ylmethoxymethyl, 2-pyrimidin-2-ylpropoxymethyl, tetrahydrofuran-2-ylmethoxymethyl, tetrahydrofuran-3-ylmethoxymethyl, tetrahydro-2H-pyran-2-ylmethoxymethyl, tetrahydro-2H-pyran-4-ylmethoxymethyl, tetrahydrothien-2-ylmethoxymethyl and tetrahydrothien-3-ylmethoxymethyl.

The term “heterocyclealkyl” as used herein, means a heterocycle group, as defined herein, appended to the parent molecular moiety through an alkyl group, as defined herein. Representative examples of heterocyclealkyl include, but are not limited to, 1,3-benzodioxol-4-ylmethyl, pyridin-3-ylmethyl, 2-pyrimidin-2-ylpropyl, tetrahydrofuran-2-ylmethyl, tetrahydrofuran-3-ylmethyl, tetrahydro-2H-pyran-2-ylmethyl, tetrahydro-2H-pyran-4-ylmethyl, tetrahydrothien-2-ylmethyl and tetrahydrothien-3-ylmethyl.

The term “heterocyclealkylthio” as used herein, means a heterocyclealkyl group, as defined herein, appended to the parent molecular moiety through a sulfur atom. Representative examples of heterocyclealkylthio include, but are not limited to, 1,3-benzodioxol-4-ylmethylthio, pyridin-3-ylmethylthio, 2-pyrimidin-2-ylpropylthio, tetrahydrofuran-2-ylmethylthio, tetrahydrofuran-3-ylmethylthio, tetrahydro-2H-pyran-2-ylmethylthio, tetrahydro-2H-pyran-4-ylmethylthio, tetrahydrothien-2-ylmethylthio and tetrahydrothien-3-ylmethylthio.

The term “heterocyclealkylthioalkyl” as used herein, means a heterocyclealkylthio group, as defined herein, appended to the parent molecular moiety through an alkyl group, as defined herein. Representative examples of heterocyclealkylthioalkyl include, but are not limited to, 1,3-benzodioxol-4-ylmethylthiomethyl, pyridin-3-ylmethylthiomethyl, 2-pyrimidin-2-ylpropylthiomethyl, tetrahydrofuran-2-ylmethylthiomethyl, tetrahydrofuran-3-ylmethylthiomethyl, tetrahydro-2H-pyran-2-ylmethylthiomethyl, tetrahydro-2H-pyran-4-ylmethylthiomethyl, tetrahydrothien-2-ylmethylthiomethyl and tetrahydrothien-3-ylmethylthiomethyl.

The term “heterocyclecarbonyl” as used herein, means a heterocycle group, as defined herein, appended to the parent molecular moiety through a carbonyl group, as defined herein. Representative examples of heterocyclecarbonyl include, but are not limited to, 1,3-benzodioxol-4-ylcarbonyl, pyridin-3-ylcarbonyl, pyrimidin-2-ylcarbonyl, tetrahydrofuran-2-ylcarbonyl, tetrahydrofuran-3-ylcarbonyl, tetrahydro-2H-pyran-2-ylcarbonyl, tetrahydro-2H-pyran-4-ylcarbonyl, tetrahydrothien-2-ylcarbonyl and tetrahydrothien-3-ylcarbonyl.

The term “heterocycleoxy” as used herein, means a heterocycle group, as defined herein, appended to the parent molecular moiety through an oxygen atom. Representative examples of heterocycleoxy include, but are not limited to, 1,3-benzodioxol-4-yloxy, pyridin-3-yloxy, 2-pyrimidin-2-yloxy, tetrahydrofuran-2-yloxy, tetrahydrofuran-3-yloxy, tetrahydro-2H-pyran-2-yloxy, tetrahydro-2H-pyran-4-yloxy, tetrahydrothien-2-yloxy and tetrahydrothien-3-yloxy.

The term “heterocycleoxyalkyl” as used herein, means a heterocycleoxy group, as defined herein, appended to the parent molecular moiety through an alkyl group, as defined herein. Representative examples of heterocycleoxyalkyl include, but are not limited to, 1,3-benzodioxol-4-yloxymethyl, pyridin-3-yloxymethyl, 2-pyrimidin-2-yloxymethyl, tetrahydrofuran-2-yloxymethyl, tetrahydrofuran-3-yloxymethyl, tetrahydro-2H-pyran-2-yloxymethyl, tetrahydro-2H-pyran-4-yloxymethyl, tetrahydrothien-2-yloxymethyl and tetrahydrothien-3-yloxymethyl.

The term “heterocyclethio” as used herein, means a heterocycle group, as defined herein, appended to the parent molecular moiety through a sulfur atom. Representative examples of heterocyclethio include, but are not limited to, 1,3-benzodioxol-4-ylthio, pyridin-3-ylthio, 2-pyrimidin-2-ylthio, tetrahydrofuran-2-ylthio, tetrahydrofuran-3-ylthio, tetrahydro-2H-pyran-2-ylthio, tetrahydro-2H-pyran-4-ylthio, tetrahydrothien-2-ylthio and tetrahydrothien-3-ylthio.

The term “heterocyclethioalkyl” as used herein, means a heterocyclethio group, as defined herein, appended to the parent molecular moiety through an alkyl group, as defined herein. Representative examples of heterocyclethioalkyl include, but are not limited to, 1,3-benzodioxol-4-ylthiomethyl, pyridin-3-ylthiomethyl, 2-pyrimidin-2-ylthiomethyl, tetrahydrofuran-2-ylthiomethyl, tetrahydrofuran-3-ylthiomethyl, tetrahydro-2H-pyran-2-ylthiomethyl, tetrahydro-2H-pyran-4-ylthiomethyl, tetrahydrothien-2-ylthiomethyl and tetrahydrothien-3-ylthiomethyl.

The term “hydroxy” as used herein, means an —OH group.

The term “hydroxyalkyl” as used herein, means at least one hydroxy group, as defined herein, appended to the parent molecular moiety through an alkyl group, as defined herein. Representative examples of hydroxyalkyl include, but are not limited to, 2-hydroxyethyl, 2-hydroxypropyl, 1,2-dihydroxypropyl, 3-hydroxybutyl and the like.

The term “hydroxyhaloalkyl” as used herein, means at least one hydroxy group, as defined herein, appended to the parent molecular moiety through a haloalkyl group, as defined herein.

The term “R_{C}R_{D}N—” as used herein, means two groups, R_{C} and R_{D}, which are appended to the parent molecular moiety through a nitrogen atom. R_{C} and R_{D} are each independently a member selected from the group consisting of hydrogen, alkoxyalkyl, alkoxyalkylcarbonyl, alkoxycarbonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (NR_{E}R_{F})alkyl and (NR_{E}R_{F})carbonyl,

The term “(R_{C}R_{D}N)alkyl” as used herein, means a R_{C}R_{D}N— group, as defined herein, appended to the parent molecular moiety through an alkyl group, as defined herein. Representative examples of (R_{C}R_{D}N)alkyl include, but are not limited to, aminomethyl, methylaminomethyl, acetylaminomethyl, acetylmethylaminomethyl, benzylaminomethyl, benzyl(methyl)amino, dimethylaminomethyl, ethylaminomethyl, diethylaminomethyl, cyclohexylaminomethyl, cyclohexylmethylaminomethyl, butylaminomethyl, 3-methylphenylaminomethyl and phenylaminomethyl.

The term “(R_{C}R_{D}N)carbonyl” as used herein, means a R_{C}R_{D}N— group, as defined herein, appended to the parent molecular moiety through a carbonyl group, as defined herein.

The term “(R_{C}R_{D}N)carbonylalkenyl” as used herein, means a (R_{C}R_{D}N)carbonyl group, as defined herein, appended to the parent molecular moiety through an alkenyl group, as defined herein.

The term “(R_{C}R_{D}N)carbonylalkyl” as used herein, means a (R_{C}R_{D}N)carbonyl group, as defined herein, appended to the parent molecular moiety through an alkyl group, as defined herein.

The term “(R_{C}R_{D}N)sulfonyl” as used herein, means a (R_{C}R_{D}N) group, as defined herein, appended to the parent molecular moiety through a sulfonyl group, as defined herein.

The term “(R_{C}R_{D}N)sulfonylalkyl” as used herein, means a (R_{C}R_{D}N)sulfonyl group, as defined herein, appended to the parent molecular moiety through an alkyl group, as defined herein.

The term “R_{E}R_{F}N—” as used herein, means two groups, R_{E} and R_{F}, which are appended to the parent molecular moiety through a nitrogen atom. R_{E} and R_{F} are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl.

The term “(R_{E}R_{F}N)alkyl” as used herein, means a R_{E}R_{F}N— group, as defined herein, appended to the parent molecular moiety through an alkyl group, as defined herein. Representative examples of (R_{E}R_{F}N)alkyl include, but are not limited to, aminomethyl, methylaminomethyl, acetylaminomethyl, acetylmethylaminomethyl, benzylaminomethyl, butylaminomethyl, 3-methylphenylaminomethyl and phenylaminomethyl.

The term “(R_{E}R_{F}N)carbonyl” as used herein, means a R_{E}R_{F}N— group, as defined herein, appended to the parent molecular moiety through a carbonyl group, as defined herein. Representative examples of (R_{E}R_{F}N)carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl and phenylaminocarbonyl.

The term “(R_{E}R_{F}N)carbonylalkyl” as used herein, means a (R_{E}R_{F}N)carbonyl group, as defined herein, appended to the parent molecular moiety through an alkyl group, as defined herein. Representative examples of (R_{E}R_{F}N)carbonylalkyl include, but are not limited to, aminocarbonylmethyl, methylaminocarbonylmethyl, acetylaminocarbonylmethyl, acetylmethylaminocarbonylmethyl, 2-(benzylaminocarbonyl)ethyl, 2-(butylaminocarbonyl)ethyl, 2-(3-methylphenylaminocarbonyl)ethyl and 2-(phenylaminocarbonyl)ethyl.

The term “R_{G}R_{H}N—” as used herein, means two groups, R_{G} and R_{H}, which are appended to the parent molecular moiety through a nitrogen atom. R_{G} and R_{H} are each independently a member selected from the group consisting of hydrogen, alkenyl, alkenyloxy, akoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, haloalkyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (R_{J}R_{K}N)alkyl and (R_{J}R_{K}N)carbonyl. Representative examples of R_{G}R_{H}N— include, but are not limited to, amino, methylamino, acetylamino, acetylmethylamino, benzylamino, (2-(benzyloxy)ethyl)amino, butylamino, cyclohexylmethylamino, cycloheptylamino, dimethylamino, ethylamino, (1-ethylpropyl)amino, isobutylamino, 3-methylphenylamino, neopentylamino, 4-nitrobenzylamino, 4-nitrophenylamino, (2-(4-nitrophenyl)ethyl)amino, phenylamino, propylamino, propylaminocarbonylamino, propionylamino, (1,3-benzodioxol-4-ylmethyl)amino, (butoxyacetyl)amino, 4-chlorobenzylamino, (4-chlorobenzyl)acetylamino, (4-chlorobenzyl)formylamino, (4-chlorobenzyl)methylamino, (1-(4-chlorophenyl)ethyl)amino, (2-(4-chlorophenyl)ethyl)amino, 2-chloropyridin-4-ylmethylamino, 6-chloropyridin-3-ylmethylamino, cyclopropylmethylamino, 3,4-dichlorobenzylamino, 4-cyanobenzylamino, (4-cyanobenzyl)methylamino, 4-cyanophenylamino, (1-(4-cyanophenyl)ethyl)amino, 2-(cyano)pyridin-4-ylmethylamino, 5-(cyano)pyridin-2-ylmethylamino, 6-(cyano)pyridin-3-ylmethylamino, (2-(tert-butoxycarbonylamino)ethyl)amino, fur-3-ylmethylamino, 4-methoxybenzylamino, tetrahydrofuran-3-ylmethylamino, tetrahydro-2H-pyran-4-ylmethylamino, (4-chlorophenylcarbonyl)amino, pyridin-2-ylmethylamino, pyridin-3-ylmethylamino, pyridin-4-ylmethylamino, (1-(pyridin-4-yl) ethyl) amino, pyrimidin-5-ylmethylamino, 1H-imidazol-4-ylmethylamino, 1H-imidazol-2-ylmethylamino, thien-2-ylmethylamino, thien-3-ylmethylamino, 4-(trifluoromethoxy)benzylamino and 6-(trifluoromethyl)pyridin-3-ylmethylamino.

The term “(R_{G}R_{H}N)alkyl” as used herein, means a R_{G}R_{H}N— group, as defined herein, appended to the parent molecular moiety through an alkyl group, as defined herein. Representative examples of (R_{G}R_{H}N)alkyl include, but are not limited to, aminomethyl, methylaminomethyl, acetylaminomethyl, acetylmethylaminomethyl, benzylaminomethyl, (2-(benzyloxy)ethyl)aminomethyl, butylaminomethyl, cyclohexylmethylaminomethyl, cycloheptylaminomethyl, dimethylaminomethyl, ethylaminomethyl, (1-ethylpropyl)aminomethyl, isobutylaminomethyl, 3-methylphenylaminomethyl, neopentylaminomethyl, 4-nitrobenzylaminomethyl, 4-nitrophenylaminomethyl, (2-(4-nitrophenyl)ethyl)aminomethyl, phenylaminomethyl, propylaminomethyl, propylaminocarbonylaminomethyl, propionylaminomethyl, (1,3-benzodioxol-4-ylmethyl)aminomethyl, (butoxyacetyl)aminomethyl, 4-chlorobenzylaminomethyl, (4-chlorobenzyl)acetylaminomethyl, (4-chlorobenzyl)formylaminomethyl, (4-chlorobenzyl)methylaminomethyl, (1-(4-chlorophenyl)ethyl)aminomethyl, (2-(4-chlorophenyl)ethyl)aminomethyl, 2-chloropyridin-4-ylmethylaminomethyl, 6-chloropyridin-3-ylmethylaminomethyl, cyclopropylmethylaminomethyl, 3,4-dichlorobenzylaminomethyl, 4-cyanobenzylaminomethyl, (4-cyanobenzyl)methylaminomethyl, 4-cyanophenylaminomethyl, (1-(4-cyanophenyl)ethyl)aminomethyl, 2-(cyano)pyridin-4-ylmethylaminomethyl, 5-(cyano)pyridin-2-ylmethylaminomethyl, 6-(cyano)pyridin-3-ylmethylaminomethyl, (2-(tert-butoxycarbonylamino)ethyl)aminomethyl, fur-3-ylmethylaminomethyl, 4-methoxybenzylaminomethyl, tetrahydrofuran-3-ylmethylaminomethyl, tetrahydro-2H-pyran-4-ylmethylaminomethyl, (4-chlorophenylcarbonyl)aminomethyl, pyridin-2-ylmethylaminomethyl, pyridin-3-ylmethylaminomethyl, pyridin-4-ylmethylaminomethyl, (1-(pyridin-4-yl)ethyl)aminomethyl, pyrimidin-5-ylmethylaminomethyl, 1H-imidazol-4-ylmethylaminomethyl, 1H-imidazol-2-ylmethylaminomethyl, thien-2-ylmethylaminomethyl, thien-3-ylmethylaminomethyl, 4-(trifluoromethoxy)benzylaminomethyl and 6-(trifluoromethyl)pyridin-3-ylmethylaminomethyl.

The term “(R_{G}R_{H}N)carbonyl” as used herein, means a —NR_{G}R_{H} group, as defined herein, appended to the parent molecular moiety through a carbonyl group, as defined herein. Representative examples of (R_{G}R_{H}N)carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl and phenylaminocarbonyl.

The term “(R_{G}R_{H}N)sulfonyl” as used herein, means a —NR_{G}R_{H} group, as defined herein, appended to the parent molecular moiety through a sulfonyl group, as defined herein. Representative examples of (R_{G}R_{H}N)sulfonyl include, but are not limited to, aminosulfonyl and dimethylaminosulfonyl.

The term “R_{J}R_{K}N—” as used herein, means two groups, R_{J} and R_{K}, which are appended to the parent molecular moiety through a nitrogen atom. R_{J} and R_{K} are each independently a member selected from the group consisting of hydrogen, alkoxycarbonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl;. Representative examples of R_{J}R_{K}N— include, but are not limited to, amino, ethylamino, benzylamino, dimethylamino, methylamino, tert-butoxycarbonylamino and propylamino.

The term “(R_{J}R_{K}N)alkyl” as used herein, means a —R_{J}R_{K}N— group, as defined herein, appended to the parent molecular moiety through an alkyl group, as defined herein. Representative examples of (R_{J}R_{K}N)alkyl include, but are not limited to, 2-aminoethyl, 2-(dimethylamino)ethyl, 2-ethylaminoethyl and 2-tert-butoxycarbonylamino)ethyl.

The term “(R_{J}R_{K}N)carbonyl” as used herein, means a —R_{J}R_{K}N— group, as defined herein, appended to the parent molecular moiety through a carbonyl group, as defined herein. Representative examples of (R_{J}R_{K}N)carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl, propylaminocarbonyl and phenylaminocarbonyl.

The term “R_{M}R_{N}N—” as used herein, means two groups, R_{M} and R_{N}, which are appended to the parent molecular moiety through a nitrogen atom. R_{M} and R_{N} are each independently a member selected from the group consisting of hydrogen, alkoxycarbonyl, alkyl, alkylcarbonyl, alkoxysulfonyl, alkylsulfonyl, aryl, arylalkyl and formyl. Representative examples of R_{M}R_{N}N— include, but are not limited to, acetylamino, amino, ethylamino, dimethylamino, methylamino and propylamino.

The term “(R_{M}R_{N}N)alkyl” as used herein, means a R_{M}R_{N}N— group, as defined herein, appended to the parent molecular moiety through an alkyl group, as defined herein.

The term “(R_{M}R_{N}N)carbonyl” as used herein, means a R_{M}R_{N}N— group, as defined herein, appended to the parent molecular moiety through a carbonyl group, as defined herein. Representative examples of (R_{M}R_{N}N)carbonyl include, but are not limited to, aminocarbonyl, methylaminocarbonyl, acetylaminocarbonyl, acetylmethylaminocarbonyl, butylaminocarbonyl and propylaminocarbonyl.

The term “(R_{M}R_{N}N)sulfonyl” as used herein, means a R_{M}R_{N}N— group, as defined herein, appended to the parent molecular moiety through a sulfonyl group, as defined herein.

The term “mercapto” as used herein, means a —SH group.

The term “nitro” as used herein, means a —NO₂ group.

The term “sulfinyl” as used herein, means a —SO— group.

The term “sulfonyl” as used herein, means a —SO₂— group.

The present compounds can exist as therapeutically suitable salts. The term “therapeutically suitable salt,” refers to salts or zwitterions of the compounds which are water or oil-soluble or dispersible, suitable for treatment of disorders without undue toxicity, irritation and allergic response, commensurate with a reasonable benefit/risk ratio and effective for their intended use. The salts can be prepared during the final isolation and purification of the compounds or separately by reacting an amino group of the compounds with a suitable acid. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetic, trifluoroacetic, glutamate, para-toluenesulfonate, undecanoate, hydrochloric, hydrobromic, sulfuric, phosphoric and the like. The amino groups of the compounds can also be quaternized with alkyl chlorides, bromides and iodides such as methyl, ethyl, propyl, isopropyl, butyl, lauryl, myristyl, stearyl and the like.

Basic addition salts can be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributlyamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine and the like, are contemplated as being within the scope of the present invention.

The present compounds can also exist as therapeutically suitable prodrugs. The term “therapeutically suitable prodrug,” refers to those prodrugs or zwitterions which are suitable for use in contact with the tissues of patients without undue toxicity, irritation and allergic response, are commensurate with a reasonable benefit/risk ratio and are effective for their intended use. The term “prodrug,” refers to compounds which are rapidly transformed in vivo to the parent compounds of the present invention for example, by hydrolysis in blood.

Asymmetric centers can exist in the present compounds. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described hereinbelow and resolved by techniques well-known in the art.

Geometric isomers can exist in the present compounds. The invention contemplates the various geometric isomers and mixtures thereof resulting from the disposal of substituents around a carbon-carbon double bond, a cycloalkyl group, or a heterocycle group. Substituents around a carbon-carbon double bond are designated as being of Z or E configuration and substituents around a cycloalkyl or heterocycle are designated as being of cis or trans configuration.

Therapeutic compositions of the present compounds comprise an effective amount of the same formulated with one or more therapeutically suitable excipients. The term “therapeutically suitable excipient,” as used herein, represents a non-toxic, solid, semi-solid or liquid filler, diluent, encapsulating material, or formulation auxiliary of any type. Examples of therapeutically suitable excipients include sugars; cellulose and derivatives thereof; oils; glycols; solutions; buffering, coloring, releasing, coating, sweetening, flavoring and perfuming agents; and the like. These therapeutic compositions can be administered parenterally, intracisternally, orally, rectally, or intraperitoneally.

Liquid dosage forms for oral administration of the present compounds comprise formulations of the same as emulsions, microemulsions, solutions, suspensions, syrups and elixirs. In addition to the compounds, the liquid dosage forms can contain diluents and/or solubilizing or emulsifying agents. Besides inert diluents, the oral compositions can include wetting, emulsifying, sweetening, flavoring and perfuming agents.

Injectable preparations of the present compounds comprise sterile, injectable, aqueous and oleaginous solutions, suspensions or emulsions, any of which can be optionally formulated with parenterally suitable diluents, dispersing, wetting, or suspending agents. These injectable preparations can be sterilized by filtration through a bacterial-retaining filter or formulated with sterilizing agents which dissolve or disperse in the injectable media.

Regulation of the effects of ghrelin by the compounds of the present invention can be delayed by using a liquid suspension of crystalline or amorphous material with poor water solubility. The rate of absorption of the compounds depends upon their rate of dissolution which, in turn, depends on their crystalline form. Delayed absorption of a parenterally administered compound can be accomplished by dissolving or suspending the compound in oil. Injectable depot forms of the compounds can also be prepared by microencapsulating the same in biodegradable polymers. Depending upon the ratio of compound to polymer and the nature of the polymer employed, the rate of release can be controlled. Depot injectable formulations are also prepared by entrapping the compounds in liposomes or microemulsions which are compatible with body tissues.

Solid dosage forms for oral administration of the present compounds include capsules, tablets, pills, powders and granules. In such forms, the compound is mixed with at least one inert, therapeutically suitable excipient such as a carrier, filler, extender, disintegrating agent, solution retarding agent, wetting agent, absorbent, or lubricant. With capsules, tablets and pills, the excipient can also contain buffering agents. Suppositories for rectal administration can be prepared by mixing the compounds with a suitable nonirritating excipient which is solid at ordinary temperature but fluid in the rectum.

The present compounds can be micro-encapsulated with one or more of the excipients discussed previously. The solid dosage forms of tablets, dragees, capsules, pills and granules can be prepared with coatings and shells such as enteric and release-controlling. In these forms, the compounds can be mixed with at least one inert diluent and can optionally comprise tableting lubricants and aids. Capsules can also optionally contain opacifying agents which delay release of the compounds in a desired part of the intestinal tract.

Transdermal patches have the added advantage of providing controlled delivery of the present compounds to the body. Such dosage forms are prepared by dissolving or dispensing the compounds in the proper medium. Absorption enhancers can also be used to increase the flux of the compounds across the skin and the rate of absorption can be controlled by providing a rate controlling membrane or by dispersing the compounds in a polymer matrix or gel.

Disorders that may be regulated by ghrelin are treated or prevented in a patient by administering to the patient, a therapeutically effective amount of a compound of the present invention in such an amount and for such time as is necessary to achieve the desired result. The term “therapeutically effective amount,” refers to a sufficient amount of a compound to effectively emeliorate disorders reglulated by ghrelin at a reasonable benefit/risk ratio applicable to any medical treatment. The specific therapeutically effective dose level for any particular patient will depend upon a variety of factors including the disorder being treated and the severity of the disorder; the activity of the compound employed; the specific composition employed; the age, body weight, general health, sex and diet of the patient; the time of administration, route of administration, rate of excretion; the duration of the treatment; and drugs used in combination or coincidental therapy.

The total daily dose of the present compounds in single or divided doses can be in amounts, for example, from 0.01 to 50 mg/kg body weight or more usually from 0.1 to 25 mg/kg body weight. In general, treatment regimens comprise administration to a patient in need of such treatment from about 10 mg to about 1000 mg of the compounds per day in single or multiple doses.

### Determination of Biological Activity

Primary Radiolabeled Ligand Competition Binding Assay

Ghrelin binding assays were performed with membrane preparations. CHO-K cells expressing human ghrelin receptor (Euroscreen) were suspended in sucrose buffer (0.25 M sucrose, 10 mM hepes pH 7.4, 1 mM PMSF, 5 μg/mL pepstain-A, 3 mM EDTA and 0.025% bacitracin) and disrupted by sonication using a vibra cell (Sonics and Materials Inc.) on 70% duty cycle in 15-second pulses on ice for 2.5 min. The homogenate was centrifuged at 60,000×g for 60 minutes and pellets were suspended in tris buffer (20 mM tris pH 7.4, 5 μg/mL pepstatin-A, 0.1 mM PMSF and 3 mM EDTA ). Binding reactions contained 1 μg membrane as determined by BCA protein assay (Pierce), 0.1 nM [¹²⁵I]-ghrelin (PerkinElmer) with or without compound addition in 100 μL of binding buffer (25 mM Hepes pH 7.4, 1 mM CaCl₂, 5 mM MgSO₄ and 0.5% protease free BSA). Incubations were carried out at room temperature for 2 hr and were terminated by filtration using Filtermate Harvester (PerkinElmer) onto GF/C filter plates (Millipore) previously soaked in 0.5% polyethylenimine for 2 hours. Bound [¹²⁵I]-ghrelin was determined by scintillation counting using Top Count NXT (PerkinElmer). The effects of compound were expressed as % inhibiton of [¹²⁵I]-ghrelin binding. Sigmoidal curves were fitted by Assay Explorer (MDL) software and IC₅₀ values were determined. GHS-R antagonist [D-Lys3]-GHRP-6 (H-His-D-Trp-D-Lys-Trp-D-Phe-Lys) was purchased from Bachem and used as a positive control.

The compounds of the present invention were found to inhibit the binding of radio-labeled ghrelin to ghrelin receptor with IC₅₀ in a range of about 0.0001 μM to about 10 μM in the binding assay. In a preferred range, the compounds inhibit the binding of radio-labeled ghrelin to ghrelin receptor with IC₅₀ in a range of about 0.0001 μM to about 1.0 μM; In a more preferred range, the compounds inhibit the binding of radio-labeled ghrelin to ghrelin receptor with IC₅₀ in a range of about 0.0001 μM to about 0.1 μM.

Secondary Fluorescent Calcium Indicator Assay (FLIPR)

CHO-K cells expressing human GHS receptor (Euroscreen) were plated in black 96-well plates with clear bottom (Costar) and cultured to confluency overnight in growth media (Ultra-CHO from BioWhittaker supplemented with 1% dialyzed FCS, 1% penicillin /streptomycin/fungizone, and 400 μg/mL G418 all from Life Technologies) at 37° C. in a humidified cell incubator containing 5% CO₂. Growth media was aspirated and replaced with 100 μL of Dulbecco's phosphate-buffered saline (DPBS) containing 1,000 mg/L D-glucose, 36 mg/L sodium pyruvate, without phenol red (Life Technologies) with 1.14 mM Fluo-4 AM (Molecular Probes) and 0.25 M probenecid (Sigma) for 1 to 3 hours in the dark at room temperature. The dye solution was aspirated and the cells were washed twice with DPBS using the EL,450X cell washer (BioTech). After the last wash, 100 μL of DPBS was added to each well. Cell plates were then transferred to the FLIPR unit (Molecular Probes). Compound additions were 50 μL in duplicate of 4× final concentration in DPBS containing 0.1% BSA and 4% DMSO. Fluorescence emissions from 96 wells were measured simultaneously at excitation and emission wavelength of 488 and 520 nm, respectively for 3 minutes in 1-second intervals for the first minute and 5-second intervals thereafter. During this time agonist responses, if any, were recorded in the absence of ghrelin. Next, 50 μL in duplicate of 4×concentrated ghrelin (Bachem) solution in DPBS containing 0.1% BSA and 4% DMSO were delivered within 1 second by an integrated 96-well pipettor to a final concentration of 1 nM. Fluorescence emissions were measured for another 3 minutes as above. During this time the antagonist effects of compounds on ghrelin-stimulated calcium flux were recorded and expressed as % inhibition of the maximal ghrelin response (10 nM). Sigmoidal curves were fitted-by Assay Explorer (MDL) software and IC₅₀ values determined. GHS-R antagonist [D-Lys3]-GHRP-6 (H-His-D-Trp-D-Lys-Trp-D-Phe-Lys) was purchased from Bachem and used as a positive control.

For the antagonists of ghrelin receptor, the compounds of the present invention were found to inhibit the activition of ghrelin receptor with IC₅₀ in a range of about 0.001 μM to about 10 μM in the FLIPR assays. In a preferred range, the compounds inhibit the activition of ghrelin receptor with IC₅₀ in a range of about 0.001 μM to about 1.0 μM; In a more preferred range, the compounds inhibit the activition of ghrelin receptor with IC₅₀ in a range of about 0.001 μM to about 0.1 μM.

Dihydrofolate Reductase (DHFR) Inhibition Assay

Human DHFR inhibition was assayed colorimetrically by following the nonenzymatic reduction of MTS (3-[4,5-dimethylthiazol-2-yl]-5-(3-carboxymethoxyphenyl]-2-[4-sulfophenyl-2-H-tetrazolium, inner salt), by tetrahydrofolate, to a soluble formazan. The final assay mix (total volume of 200 μL) included potassium phosphate buffer (66 mM, pH 7.0), potassium chloride (150 mM), EDTA (1.2 mM), 2-mercaptoethanol (1 mM), NADPH (40 μM), MTS (0.025 mg/mL), dihydrofolate (30 μM), BSA (0.1 mg/mL), 1% DMSO, and 0.47 μg/mL human DHFR (Sigma, St. Louis, Mo.). A reagent mix, including the assay buffer (EDTA+KPO4+KCl), 2-mercaptoethanol, NADPH, and MTS was combined, protected from light, kept on ice, and added to the test plate via a Titertek Multidrop 384. DHFR was added using a Beckman Coulter Multimek 96. The reaction was initiated with the addition of the final reagent, dihydrofolate (FAH₂), using a Multimek 96. A chiller block was used on a Multimek in a darkened room in order to prevent enzyme and substrate degradation. The test plate was then immediately transferred to a Molecular Devices Spectramax Plus 384 and read kinetically at 490 nm over 2 minutes. The IC₅₀ is determined based upon the amount of drug that inhibits the rate by 50% of the control without drug. Methotrexate (Sigma, St. Louis, Mo.) was used as the positive drug control in this study.

TABLE 1
Functional Activity of Representative Ghrelin Antagonists and
Their Selectivity against Human Dihydrofolate Reductase
(hDHFR)

| | | Percentage | Percentage | Percentage |
|---|---|---|---|---|
| | h-GHS-R | of Inhibition | of Inhibition | of Inhibition |
| | FLIPR | of hDHFR | of hDHFR | of hDHFR |
| Compound | IC₅₀ (μM) | at 1.0 μM | at 10.0 μM | at 30.0 μM |
| A | 0.0093 | 9.0 | 5.0 | ND |
| B | 0.21 | 1.4 | 6.0 | ND |
| C | 0.014 | ND | 28.5 | 11.3 |
| D | 0.119 | ND | 13.4 | 6.2 |
| E | 0.0096 | −3.7 | −2.3 | ND |
| F | 0.015 | 10.0 | 0.0 | ND |
| G | 0.022 | 4.0 | 9.0 | ND |
| H | 0.035 | 1.0 | 9.5 | ND |
| I | 0.010 | −10.3 | −1.5 | ND |

ND: not determined.

The results shown in Table 1 clearly demonstrate that Compounds A, B, C, D, E, F, G, H and I are at least 100 fold selective for ghrelin receptor over dihydrofolate reductase. Moreover, the compounds of the present invention were found to antagonize the function of ghrelin in a range of about 0.001 μM to about 0.1 μM without inhibiting human dihydrofolate reductase (hDHFR).
Effect of Ghrelin Antagonist on Weight Loss in Diet-Induced Obese Mice

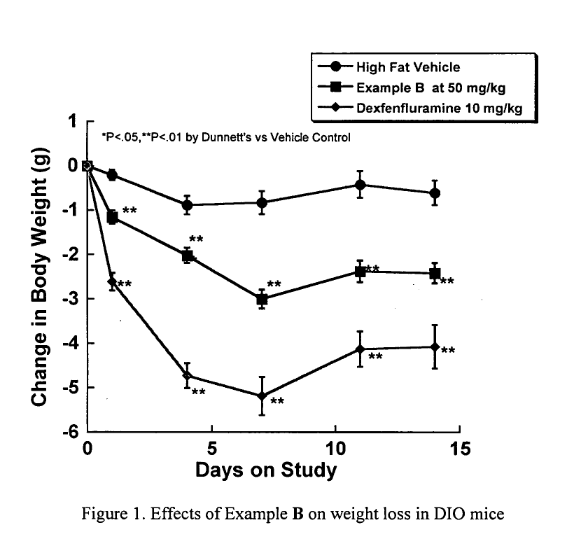

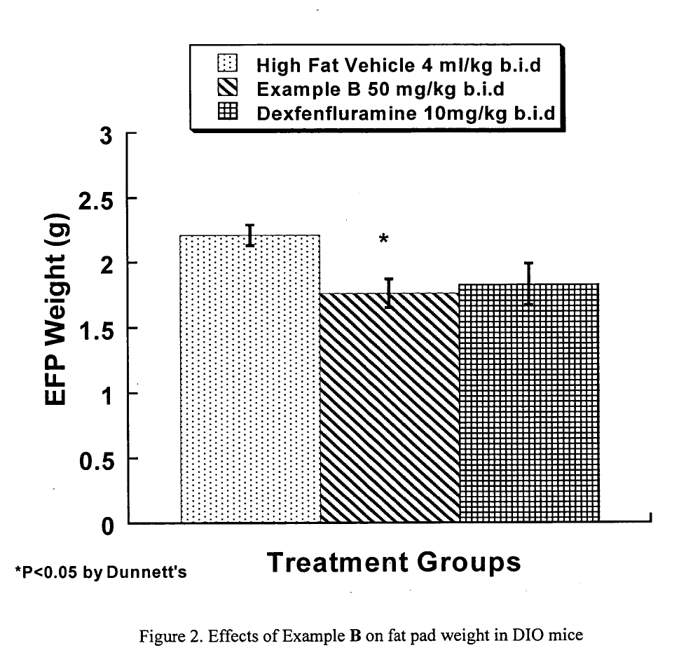

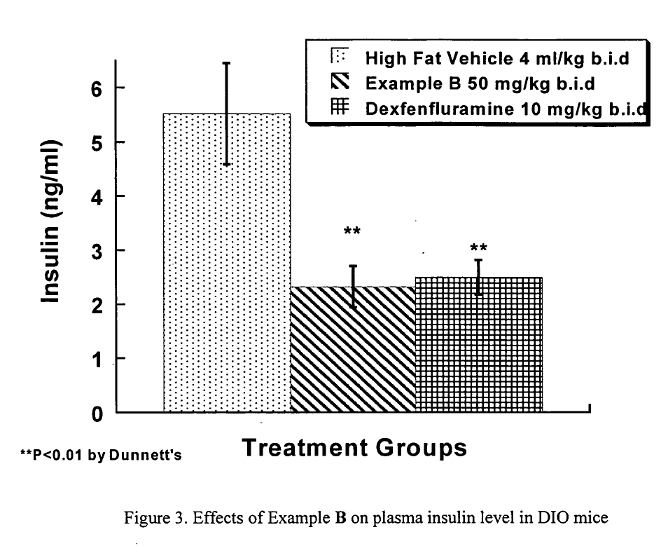

Diet-induced obese male C57BL/6J mice (Jackson Lab, 25 weeks of age) were dosed orally twice a day either a vehicle or Example B at 50 mg/kg at approx 08:00 and 15:00 h. Dexfenfluramine dosed at 10 mg/kg, p.o., b.i.d was used as the positive control. Body weight and food intake were monitored periodically throughout the study. After 2 weeks of drug treatment, mice were euthanized. As shown in FIG. 1, the drug treatment with Example B resulted in a body weight loss of 5.4% in comparison to vehicle treated mice by the end of the two week study. As shown in FIG. 2, drug treatment with Example B resulted in significantly decreased epididymal fat pad weight. Finally, as shown in FIG. 3, the drug treatment with Example B significantly lowered plasma insulin level at the end of two-week study.

Synthetic Methods

Abbreviations which have been used in the descriptions of the scheme and the examples that follow are: BBr₃ for boron tribromide; m-CPBA for meta-chloroperoxy-benzoic acid; DMF for N,N-dimethylformamide; DMSO for dimethylsulfoxide; DEAD for diethyl azodicarboxylate; EDAC for 1-(3-dimethylaminopropyl)-3-ethylcarbodiimide hydrochloride; HATU for O-(7-azabenzotriazol-1-yl)-N,N,N′N′-tetramethyluronium hexafluorophosphate; HOBT for 1-hydroxybenzotriazole hydrate; NMP for N-methylpyrrolidinone; NCS for N-chlorosuccinimide; MeONa for sodium methoxide; MeOH for methanol; MTBE for methyl tert butyl ether; THF for tetrahydrofuran; TFA for trifluoroacetic acid; TMSCHN₂ for trimethylsilyldiazomethane; TBAF for tetra butylammonium fluoride; Pd(dppf)Cl₂ for (diphenylphospino)ferrocenyl palladium chloride; Ph₃P for triphenylphosphine; Pr₂Net for diisopropyl ethylamine; and TBTU for (benzotriazol-1-yloxy)-dimethylamino-methylene)-dimethyl-ammonium tetrafluoroborate.

The compounds and processes of the present invention will be better understood in connection with the following synthetic schemes which illustrate the methods by which the compounds of the invention may be prepared.

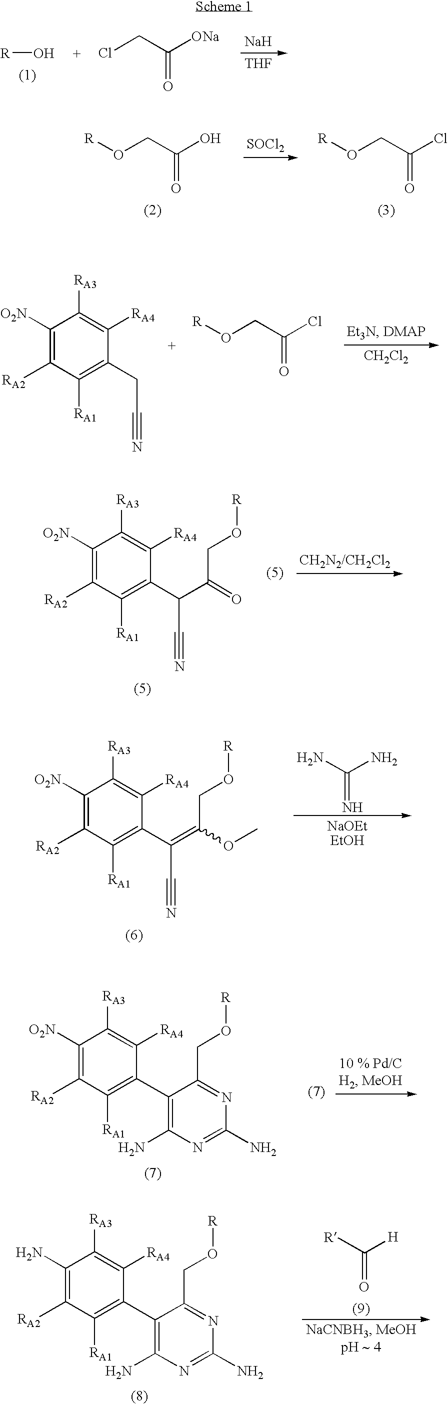

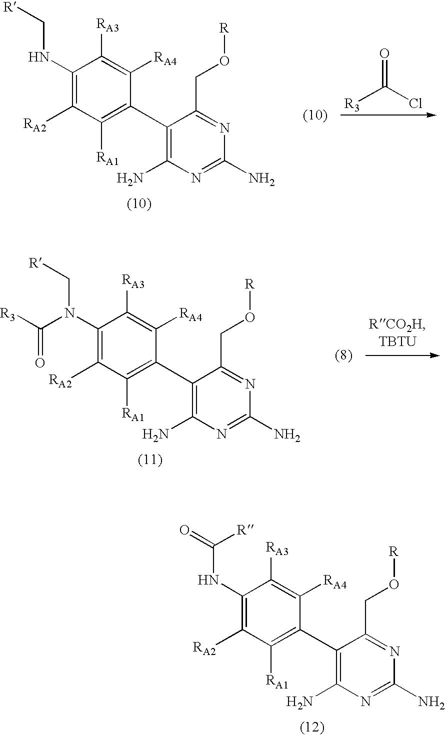

Compounds of the present invention of general formula (10), (11), (12) and (13), wherein R_{A1}, R_{A2}, R_{A3} and R_{A4},are as defined in formula (I), R is alkenyl, alkoxyalkoxyalkyl, alkoxyalkyl, alkoxycarbony, alkoxycarbonylalkyl, alkoxysulfonyl, alkylcarbonyl, alkylcarbonylalkyl, alkylthioalkyl, alkynyl, aryl, arylalkoxyalkyl, arylalkyl, arylalkylthioalkyl, aryloxyalkyl, arylthioalkyl, cyanoalkyl, cycloalkenyl, cycloalkenylalkoxyalkyl, cycloalkenylalkyl, cycloalkenylalkylthioalkyl, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, (R_{E}R_{H}N)alkyl, (R_{E}R_{H}N)carbonylalkenyl, (R_{E}R_{H}N)carbonylalkyl, (R_{E}R_{H}N)sulfonyl, or (R_{E}R_{H}N)sulfonylalkyl, R′ and R″ are each independently selected from hydrogen, alkoxyalkyl, alkyl, alkylthioalkyl, aryl, arylalkoxyalkyl, arylalkyl, cycloalkyl, cycloalkylalkoxyalkyl, cycloalkylalkyl, heteroaryl, heteroarylalkoxyalkyl, heteroarylalkyl, heterocycle, heterocyclealkoxyalkyl, or heterocyclealkyl and R_{E} and R_{F} are as defined in formula (I), can be prepared as described in Scheme 1. Phenols or alcohols of general formula (1) can be treated with sodium chloroacetate to provide acids of general formula (2). Acids of general formula (2) can be treated with thionyl chloride to provide acid chlorides of general formula (3). Acid chlorides of general formula (3) can be treated with cyano compounds of general formula (4) to provide ketones of general formula (5). Ketones of general formula (5) can be treated with diazomethane followed by treatment with guanidine to provide nitrophenylpyrimidines of general formula (7). Nitrophenylpyrimidines of general formula (7) can be reduced under conditions well known to those of skill in the art including, but not limited to, a palladium catalyst under varying atmosphere of hydrogen to provide aminophenylpyrimidines of general formula (8). Aminophenylpyrimidines of general formula (8) can be treated with aldehydes of general formula (9) (or ketones) under reductive amination conditions to provide secondary-aminophenylpyrimidines of general formula (10). Both the primary and the secondary-aminophenylpyrimidines of general formula (10) and (8) can be coupled with acids, acid chlorides, or carbonyl compounds to provide compounds of general formula (11) and (12).

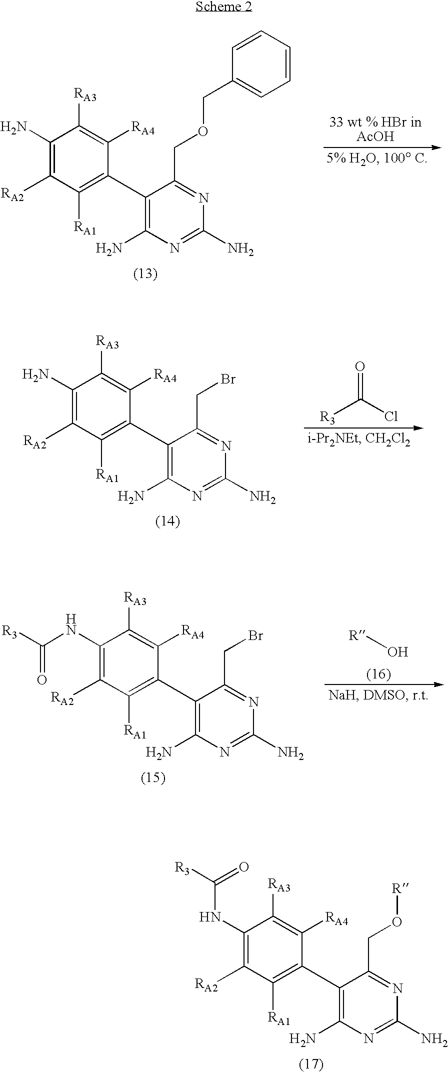

Compounds of the present invention of general formula (17) can be prepared as described in Scheme 2. Aminophenylpyrimidines of general formula (13), prepared as described in Scheme 1, can be treated with 33% wt of HBr in AcOH at 100° C. to provide diaminopyrimidine methylbromide of general formula (14). The anilino group can then be acylated under standard conditions to provide amide derivatives of general formula (15). The bromide can then be subject to standard Williamson ether synthesis to provide either compounds of general formula (17). Similarly, other nucleophiles, such as amines, mercaptans, heterocycles, heteroaryls, can also be used to displace the bromide to provide other compounds representative of the present invention.

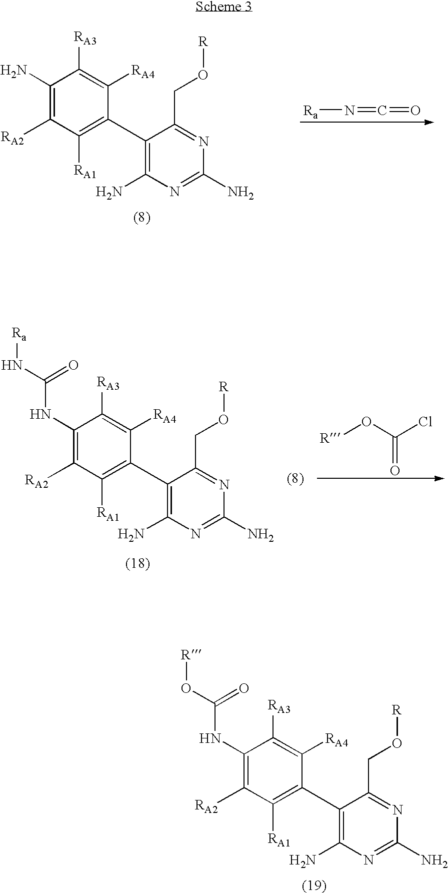

Compounds of general formula (8) can be treated with isocyanate to provide ureas of general formula (18). Compounds of general formula (8) can also be treated with chloroformate to provide carbamates of general formula (19).

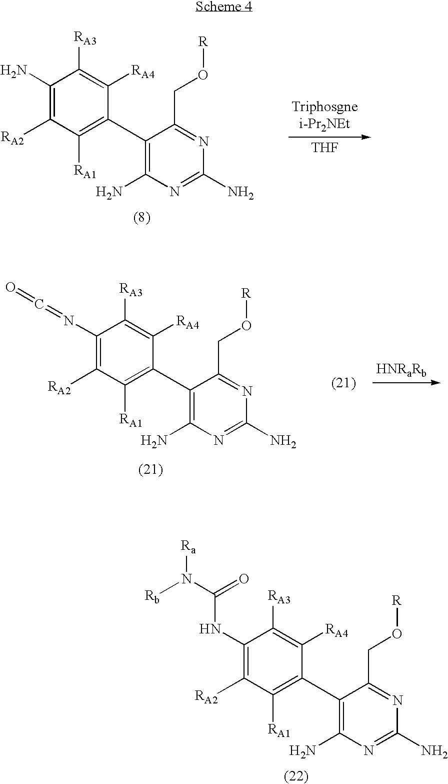

Compounds of the present invention of general formula (22) can be prepared as described in Scheme 4. Compounds of general formula (8), prepared as described in Example 1 herein, can be treated with triphosgene and i-Pr₂NEt to provide isocyanates of general formula (21). Diaminopyrimidines of general formula (21) can be treated with amines as described in Scheme 4 to provide ureas of general formula (22). Alternatively, diaminopyrimidines of general formula (21) can be treated with alcohols as described in Scheme 4 to provide ureas of general formula (23).

The present invention will now be described in connection with certain embodiments which are not intended to limit its scope. On the contrary, the present invention covers all alternatives, modifications and equivalents as can be included within the scope of the claims. Thus, the following examples, which include preferred embodiments, will illustrate the preferred practice of the present invention, it being understood that the examples are for the purposes of illustration of certain preferred embodiments.

Compounds of the invention were named by ACD/ChemSketch version 5.01 (developed by Advanced Chemistry Development, Inc., Toronto, ON, Canada) or were given names which appeared to be consistent with ACD nomenclature.

### EXAMPLE 1

### N-[4-(2,4-diamino-6-ethylpyrimidin-5-yl)phenyl]indane-2-carboxamide

### EXAMPLE 1A

### 2-(4-Nitro-phenyl)-3-oxo-pentanenitrile

To a solution of 8.10 g (50.0 mmol) of 4-nitrophenylacetonitrile in 100 mL of CH₂Cl₂ was added 610 mg (5 mmol) of 4-N,N-dimethylaminopyridine. The solution was cooled with an ice bath, then 8.7 mL (100 mmol) of propionyl chloride was added dropwise to avoid reflux of the solvent. After 45 minutes, the solvent was removed in vacuo and the residue was taken up in 200 mL of 0.5 M HCl. The mixture was extracted with diethyl ether (3×50 mL), then the combined ether layers were back extracted with water (1×50 mL), brine (1×50 mL), dried over MgSO₄, filtered and concentrated under reduced pressure to an oil.

The oil was taken up in 250 mL of methanol and to the solution was added 200 mL of 2M NaOH. The solution was stirred for 15 minutes, then 1 L of water was added, followed by 12M HCl until precipitation was complete. The suspension was extracted with diethyl ether (2×200 mL), then the combined ether layers were back extracted with brine (1×100 mL), dried over MgSO₄, filtered and concentrated under reduced pressure to provide the titled compound (9.3 g, 85%) as a solid. This material may be used in the next step without further purification, or maybe recrystallized from toluene to give a crystalline product.

### EXAMPLE 1B

### 6-Ethyl-5-(4-nitro-phenyl)-pyrimidine-2,4-diamine

To 1.91 g (8.75 mmol) of 2-(4-nitro-phenyl)-3-oxo-pentanenitrile from Example 1A in 20 mL of ethyl acetate was added ethereal CH₂N₂ until excess CH₂N₂ was present. The reaction was concentrated to an oil. This was taken up in 5 mL of ethanol, then treated with a premixed solution of 955 mg (10 mmol) of guanidine hydrochloride and potassium ethoxide (10 mmol) in 14 mL of ethanol. (The guanidine solution contained precipitated KCl). The reaction was stirred at reflux for 2 hours, then concentrated under reduced pressure. The residue was taken up in 20 mL of water and filtered to give a black precipitate. The precipitate was washed with 100 mL of water, recrystallized from 25 mL of ethanol. The recrystallized product was filtered and washed with 10 mL of cold ethanol to provide the titled compound (700 mg, 27%) as green crystals.

### EXAMPLE 1C

### 5-(4-Amino-phenyl)-6-ethyl-pyrimidine-2,4-diamine

To a solution of 1.95 g (7.52 mmol) of 6-ethyl-5-(4-nitro-phenyl)-pyrimidine-2,4-diamine from Example 1B in 60 mL of glacial acetic acid was added 200 mg of 10% Pd—C. The reaction was stirred under 1 atmosphere of H₂ for 5 hours. The catalyst was filtered and the solvent was removed under reduced pressure at 40° C. to provide a crystalline solid. The solid was dissolved in 25 mL of water and the solution was made basic (pH=14) by the addition of 2M NaOH. The precipitate was filtered and washed with water until the washings were pH=8. The product was dried on the filter to provide 1.55 g (90%) of light yellow crystals.

### EXAMPLE 1D

### N-[4-(2,4-diamino-6-ethylpyrimidin-5-yl)phenyl]indane-2-carboxamide

To a stirred mixture of aniline from Example 1C (55 mg, 0.2 mmol), indan-2-carboxylic acid (32 mg, 0.2 mmol) and Et₃N (30 mg, 0.3 mmol) in DMF (1.5 mL) was added TBTU (64 mg, 0.2 mmol). The reaction mixture was stirred at r.t overnight and purified by reverse phase preparative HPLC to give the titled compound (50 mg, 67%). ¹H NMR (300 MHz, DMSO-d₆) δ 10.11 (1H), 7.69 (d, J=8.4 Hz, 2H), 7.25-7.13 (m, 4H), 7.11 (d, J=8.4 Hz, 2H) 5.86 (s, 2H), 5.46 (s, 2H), 3.43 (quintet, J=9.0 Hz, 1H), 3.18 (d, J=8.1 Hz, 4H), 2.13 (q, 7.5 Hz, 2H) and 0.96 (t, J=7.8 Hz, 3H). MS (ESI) m/e positive ion 374 (M+H)⁺; negative ion 372 (M−H)⁺.

### EXAMPLE 2

### N-[4-(2,4-diamino-6-ethylpyrimidin-5-yl)phenyl]-N′-(2-phenylethyl)urea

To a solution of aniline from Example 1C (11.5 mg, 0.05 mmol) in THF (1 mL) was added diisopropylethylamine (17 μL, 0.1 mol), followed by triphosgene (5 mg, 0.017 mmol). The suspension was stirred at room temperature for 5 minutes. Phenethylamine was then added and stirred for 5 minutes before adding DMSO to make a clear solution. The mixture was purified by reverse phase HPLC (0-70% CH₃CN in aq. NH₄OAc) to provide the titled compound as an off-white solid (12 mg, 64%). ¹H NMR (300 MHz, DMSO-d₆) δ 8.55 (s, 1H), 7.43 (d, J=8.6 Hz, 2H), 7.36-7.18 (m, 5H), 7.01 (d, J=8.6 Hz, 2H), 6.14 (t, J=6.0 Hz, 1H), 5.78 (s. 2H), 5.35 (bs, 2H), 3.42-3.30 (m, 2H), 2.76 (t, J=7.1 Hz, 2H), 2.12 (q, J=7.5 Hz, 2H), 0.95 (t, J=7.5 Hz, 3H). MS (ESI) positive ion 377 (M+H)⁺; negative ion 375 (M−H)⁻.

### EXAMPLE 3

### N-(4-chlorobenzyl)-N′-[4-(2,4-diamino-6-ethylpyrimidin-5-yl)phenyl]urea

The titled compound was synthesized according to the procedure described in Example 2, substituting 4-chlorobenzylamine for phenethylamine. ¹H NMR (300 MHz, DMSO-d₆) δ 8.68 (s, 1H), 7.45 (d, J=8.5 Hz, 2H), 7.43-7.30 (m, 4H), 7.03 (d, J=8.5 Hz, 2H), 6.68 (t, J=6.0 Hz, 1H), 5.78 (s, 2H), 5.36 (bs, 2H), 4.30 (d, J=6.1 Hz, 2H), 2.12 (q, J=7.5 Hz, 2H), 0.95 (t, J=7.5 Hz, 3H). MS (ESI) positive ion 397 (M+H)⁺; negative ion 395 (M−H)⁻.

### EXAMPLE 4

### N-[4-(2,4-diamino-6-ethylpyrimidin-5-yl)phenyl]-N′-(1-phenylethyl)urea

The titled compound was synthesized according to the procedure described in Example 2, substituting α-methylbenzylamine for phenethylamine. ¹H NMR (300 MHz, DMSO-d₆) δ 8.45 (s, 1H), 7.42 (d, J=8.5 Hz, 2H), 7.37-7.20 (m, 5H), 7.01 (d, J=8.5 Hz, 2H), 6.62 (d, J=7.8 Hz, 1H), 5.82 (s, 2H), 5.39 (bs, 2H), 4.88-4.77 (m, 1H), 2.11 (q, J=7.5 Hz, 2H), 1.39 (d. J=7.1 Hz, 3H), 0.95 (t, J=7.5 Hz, 3H). MS (ESI) positive ion 377 (M+H)⁺; negative ion 375 (M−H)⁻.

### EXAMPLE 5

### N-[1-(4-chlorophenyl)ethyl]-N′-[4-(2,4-diamino-6-ethylpyrimidin-5-yl)phenyl]urea

The titled compound was synthesized according to the procedure described in Example 2, substituting α-methyl-4-chloro-benzylamine for phenethylamine. ¹H NMR (300 MHz, DMSO-d₆) δ 8.53 (s, 1H), 7.41 (d, J=8.5 Hz, 2H), 7.37-7.20 (m, 4H), 7.01 (d, J=8.5 Hz, 2H), 6.74 (jd, J=7.8 Hz, 1H), 5.78 (s, 2H), 5.34 (bs, 2H), 4.88-4.77 (m, 1H), 2.11 (q, J=7.5 Hz, 2H) 1.38 (d, J=7.1 Hz, 3H), 0.94 (t, J=7.5 Hz, 3H). MS (ESI) positive ion 411 (M+H)⁺; negative ion 409 (M−H)⁻.

### EXAMPLE 6

### N-[1-(4-chlorophenyl)ethyl]-N′-(4-{2,4-diamino-6-[(benzyloxy)methyl]pyrimidin-5-yl}phenyl)urea

### EXAMPLE 6A

### 4-Benzyloxy-2-(4-nitro-phenyl)-3-oxo-butyronitrile

4-Nitrophenylacetonitrile (10.0 g, 61.7 mmol), triethylamine (14.5 g, 144 mmol) and 4-(dimethylamino)pyridine (800 mg, 6.56 mmol) were dissolved in CH₂Cl₂ (150 mL). The solution was cooled to 0° C. and benzyloxyacetyl chloride (12.0 g, 64.8 mmol) was added dropwise over a 30 minutes. The reaction mixture was warmed to room temperature and stirred for 2 hours. CH₂Cl₂ was removed under reduced pressure and the mixture was dissolved in ethyl acetate (150 mL) and washed with aqueous NaHCO₃ (150 mL) and aqueous HCl (10%, 2×150 mL). The solcents were removed under reduced pressure to provide crude 4-benzyloxy-2-(4-nitro-phenyl)-3-oxo-butyronitrile (19.6 g). R_{f}=0.11 (50% ethyl acetate in hexanes).

### EXAMPLE 6B

### 6-Benzyloxymethyl-5-(4-nitro-phenyl)-pyrimidine-2,4-diamine

4-Benzyloxy-2-(4-nitro-phenyl)-3-oxo-butyronitrile (9.72 g, 31.4 mmol) from Example 6A was dissolved in CH₂Cl₂ (80 mL) and TMSCHN₂ (30 mL, 2M in Et₂O, 60 mmol) was add slowly. HOAc (glacial) was added dropwise until excess TMSCHN₂ was destroyed as evidenced by the cessation of N₂ evolution. The solution was concentrated under reduced pressure and the residue dissolved in 60 mL EtOH. Guanidine hydrochloride (3.605 g, 37.5 mmol) was mixed with 60 mL EtOH followed by addition of NaOEt in EtOH (14.2 mL, 37.6 mmol). After stirring the guanidine solution for 5 minutes the solution was added to the enol ether/ethanol solution resulting in a very dark, purple mixture. The reaction mixture was heated to reflux for 3 hours. The solution was concentrated under reduced pressure followed by addition of EtOAc (150 mL) and aqueous NaOH (200 mL, 0.5M). The mixture was stirred and the formed precipitate was filtered providing 6-benzyloxymethyl-5-(4-nitro-phenyl)-pyrimidine-2,4-diamine (8.78 g, 79.5%) as a light brown solid.

### EXAMPLE 6C

### 5-(4-Amino-phenyl)-6-benzyloxymethyl-pyrimidine-2,4-diamine

6-Benzyloxymethyl-5-(4-nitro-phenyl)-pyrimidine-2,4-diamine (5.00 g, 14.25 mmol) from Example 6B and Pd(OH)₂/C (600 mg) in MeOH (140 mL) in a heavy walled reaction vessel was charged with H₂ (60 psi) and the mixture shaken at room temperature for 14 hour. The mixture was filtered to remove the catalyst and the solution concentrated to provide 5-(4-amino-phenyl)-6-benzyloxymethyl-pyrimidine-2,4-diamine (4.34 g, 95%) as a light brown solid.

### EXAMPLE 6D

### N-[1-(4-chlorophenyl)ethyl]-N′-(4-{2,4-diamino-6-[(benzyloxy)methyl]pyrimidin-5-yl}phenyl)urea

The titled compound was synthesized according to the procedure described in Example 2, substituting aniline from Example 6C for aniline from Example 1C and α-methyl-4-chloro-benzylamine for phenethylamine. ¹H NMR (300 MHz, DMSO-d₆) δ 8.47 (s, 1H), 7.42-7.16 (m, 11H), 7.05 (d, J=8.5 Hz, 2H), 6.67 (d, J=7.8 Hz, 1H), 5.98 (s, 2H), 5.63 (bs, 2H), 4.88-4.77 (m, 1H), 4.33 (s, 2H), 3.96 (s, 2H), 1.38 (d, J=6.8Hz, 3H). MS (ESI) positive ion 503 (M+H)⁺; negative ion 501 (M−H)⁻.

### EXAMPLE 7

### 4-chloro-N-(4-{2,4-diamino-6-[(benzyloxy)methyl]pyrimidin-5-yl}phenyl)benzamide

5-(4-Amino-phenyl)-6-benzyloxymethyl-pyrimidine-2,4-diamine (50 mg, 0.16 mmol) from example 6C, 4-chlorobenzoic acid (24 mg, 0.16 mmol) and TBTU (70 mg, 0.22 mmol) were dissolved in DMF (1 mL). The mixture was stirred for 5 minutes followed by the addition of Et₃N (0.27 mL, 1.6 mmol). The reaction mixture was stirred for 2 hours at room temperature and separated by reverse phase HPLC (0-70% CH₃CN in aq. NH₄OAc) providing 4-chloro-N-[4-(2,4-diamino-6-benzyloxymethyl-pyrimidin-5-yl)-phenyl]-benzamide (25 mg, 35%) as an off-white solid. ¹H NMR (300 MHz, DMSO-d₆) δ 3.99 (s, 2 H), 4.34 (s, 2 H), 5.65 (s, 2 H), 5.99 (s, 2 H), 7.20 (m, 4 H), 7.28 (m, 3 H), 7.63 (m, 2 H), 7.83 (d, J=8.48 Hz, 2 H), 8.00 (ddd, J=8.99, 2.37, 2.20 Hz, 2 H) and 10.38 (s, 1 H). MS (ESI) positive ion 460 (M+H)⁺; negative ion 458 (M−H)³¹.

### EXAMPLE 8

### 2-butoxy-N-(4-{2,4-diamino-6-[(benzyloxy)methyl]pyrimidin-5-yl}phenyl)acetamide

The title compound was-synthesized according to the procedure described in Example 7, substituting n-butoxyacetic acid for 4-chlorobenzoic acid. ¹H NMR (300 MHz, DMSO-d₆) δ 0.91 (t, J=7.29 Hz, 3 H) 1.37 (m, 2 H) 1.58 (m, 2 H) 3.52 (t, J=6.61 Hz, 2 H) 3.98 (s, 2 H) 4.05 (s, 2 H) 4.34 (s, 2 H) 5.81 (s, 2 H) 6.12 (s, 2 H) 7.15 (d, J=8.48 Hz, 2 H) 7.16 (m, 2 H), 7.26 (m, 3 H) 7.70 (d, J=8.81 Hz, 2 H) 9.75 (s, 1 H). MS (ESI) positive ion 436 (M+H)⁺; negative ion 434 (M−H)⁻.

### EXAMPLE 9

### N-(4-{2,4-diamino-6-[(benzyloxy)methyl]pyrimidin-5-yl}phenyl)propanamide

The title compound was synthesized according to the procedure described in Example 7, substituting propionic acid for 4-chlorobenzoic acid. ¹H NMR (300 MHz, DMSO-d₆) δ 1.10 (d, J=7.60 Hz, 3 H), 2.34 (q, J=7.57 Hz, 2 H), 3.96 (s, 2 H), 4.33 (s, 2 H), 5.64 (s, 2 H), 5.99 (s. 2 H), 7.12 (d, J=8.81 Hz, 2 H), 7.15 (m, 2 H), 7.26 (m, 3 H), 7.63 (d, J=8.48 Hz, 2 H), and 9.91 (s, 1 H). MS (ESI) positive ion 378 (M+H)⁺; negative ion 376 (M−H)⁻.

### EXAMPLE 10

N-(4-chlorobenzyl)-N-(4-{2,4-diamino-6-[(benzyloxy)methyl]pyrimidin-5-yl}phenyl)acetamide

### EXAMPLE 10A

### 6-Benzyloxymethyl-5-[4-(4-chloro-benzylamino)-phenyl]-pyrimidine-2,4-diamine

5-(4-Amino-phenyl)-6-benzyloxymethyl-pyrimidine-2,4-diamine (1.62 g, 5.05 mmol) from Example 6C was dissolved in MeOH/NaOAc/HOAc (80 mL, 1M, pH 4). 4-Chlorobenzaldehyde (851 mg, 6.06 mmol) was added and the mixture stirred for 15 minutes. NaBH₃CN (375 mg, 6.06) was then added and the reaction mixture was stirred for 16 hours at 25° C. EtOAc (180 mL) was added and the mixture was washed with HCl (10%, 75 mL), NaOH (2M, 2×100 mL) and brine (100 mL). The crude material was purified by silica gel chromatography (EtOAc to 10% MeOH in EtOAc gradient) providing 6-benzyloxymethyl-5-[4-(4-chloro-benzylamino)-phenyl]-pyrimidine-2,4-diamine (1.56 g, 69.5%). ¹H NMR (300 MHz, DMSO-d₆) δ 3.94 (s, 2 H), 4.28 (d, J=5.8 Hz, 2 H), 4.32 (s, 2 H), 5.49 (s, 2 H), 6.37 (t, J=5.9 Hz, 1 H), 6.60 (d, J=8.5 Hz, 2 H), 6.89 (d, J=8.5 Hz, 2 H), 7.17 (m, 2 H), 7.27 (m, 3 H) and 7.40 (m, 4 H). MS (ESI) positive ion 446 (M+H)⁺; negative ion 444 (M−H)⁻.

### EXAMPLE 10B

### N-(4-Chlorobenzyl)-N-(4-{2,4-diamino-6-[(benzyloxy)methyl]pyrimidin-5-yl}phenyl)acetamide

To a solution of 4-chlorobenzyl(4-{2,4-diamino-6-[(benzyloxy)methyl]pyrimidin-5-yl}aniline from Example 10A (35 mg,0.08 mmol) in CH₂Cl₂ (2 mL) at 0° C. was added acetyl chloride (0.09 mmol). The mixture was stirred at 0° C. for 10 minutes, at room temperature for 0.5 hour and concentrated under reduced pressure. The residue was purified by silica gel flash column chromatography to provide the title compound (35.1 mg, 90%). ¹H NMR (300 MHz, DMSO-d₆) δ 7.34-7.14 (m, 13H), 6.0 (s, 2H), 5.72 (s, 2H), 4.84 (s, 2H), 4.24 (s, 2H), 3.90 (s, 2H), 1.87 (s, 3H). MS (ESI) positive ion 488 (M+H)⁺; negative ion 486 (M−H)⁻.

### EXAMPLE 11

### 4-chlorobenzyl(4-{2,4-diamino-6-[(benzyloxy)methyl]pyrimidin-5-yl]phenyl)formamide

A mixture of 4-chlorobenzyl(4-{2,4-diamino-6-[(benzyloxy)methyl]pyrimidin-5-yl}aniline from Example 10A ( 44.5 mg, 0.1 mmol), formic acid (140 mg, 3 mmol) and acetic anhydride (102 mg, 1 mmol) in 10 mL flask was heated at 60° C. for 1 hour. The mixture was concentrated under reduced pressure, the residue diluted with water, basified with 5% NaOH to a pH of 10 and extracted with CH₂Cl₂. The combined organic extracts were dried over MgSO4, filtered, concentrated under reduced pressure and then purified by column chromatography to provide the title compound (42 mg, 88%). ¹H NMR (300 MHz, DMSO-d₆) δ 8.69 (s, 1H), 7.36-7.12 (m, 13H), 5.98 (s, 2H), 5.63 (s, 2H), 5.02 (s, 2H), 4.25 (s, 2H), 3.90 (s, 2H). MS (ESI) positive ion 474 (M+H)⁺; negative ion 472 (M−H)⁻.

### EXAMPLE 12

### N′-allyl-N-(4-chlorobenzyl)-N-(4-{2,4-diamino-6-[(benzyloxy)methyl]pyrimidin-5-yl}phenyl)urea

To a solution of 6-benzyloxymethyl-5-[4-(4-chloro-benzylamino)-phenyl]-pyrimidine-2,4-diamine (40 mg, 0.090 mmol) from Example 10A in THF (1 mL) was added allyl isocyanate (7.45 mg, 0.090 mmol) in THF (0.1 mL) and stirred for 1 h at room temperature. LC-MS of the reaction mixture showed conversion to be low. Allyl isocyanate (7.45 mg, 0.090 mmol) in THF (0.1 mL) was then added and the reaction was stirred overnight at room temperature. The solvent was removed in vacuo and the crude mixture was dissolved in DMF (1 mL) and methanol (1 mL) to purify by RP-HPLC (5-100% CH₃CN in aqueous NH₄OAc). The titled compound (3.7 mg, 7.9%) was recovered as a white solid. ¹H NMR (300 MHz, DMSO-d₆) δ 3.68 (t, J=5.26 Hz, 2 H), 3.93 (s, 2 H), 4.26 (s, 2 H), 4.81 (s, 2 H), 4.97-5.11 (m, 2 H), 5.70-5.88 (m, 3 H), 5.88-6.03 (m, 3 H), 7.10-7.19 (m, 6 H), 7.20-7.26 (m, 5 H), 7.29-7.35 (m, 2 H). MS (ESI) positive ion 529 (M+H)⁺; negative ion 527 (M−H)⁻.

### EXAMPLE 13

### 2-(2-bromothien-3-yl)-N-(4-{2,4-diamino-6-[(benzyloxy)methyl]pyrimidin-5-yl}phenyl)acetamide

The titled compound was prepared following the same procedure as described in Example 7, substituting (2-bromo-thiophen-3-yl)-acetic acid for 4-chlorobenzoic acid used in Example 7. ¹H NMR (300 MHz, DMSO-d₆) δ 10.27 (s, 1H), 7.64 (d, J=8.5 Hz, 2H), 7.58 (d, J=5.8 Hz, 1H), 7.32-7.16 (m, 5H), 7.14 (d, J=8.5 Hz, 2H), 7.06 (d, J=5.4 Hz, 1H), 5.96 (s, 2H), 5.62 (s, 2H), 4.33 (s, 2H), 3.95 (s, 2H), 3.68 (s, 2H). MS (ESI) m/e 524, 526 (M+H)⁺.

### EXAMPLE 14

### N-(4-{2,4-diamino-6-[(benzyloxy)methyl]pyrimidin-5-yl}phenyl)-2-methylcyclopropanecarboxamide

5-(4-Amino-phenyl)-6-benzyloxymethyl-pyrimidine-2,4-diamine (22 mg, 0.07 mmol) from example 6C, 2-methylcyclopropanecarboxylic acid (9 mg, 0.09 mmol), HOBT (11.7 mg, 0.09 mmol) and DIEA (18 mg, 0.14 mmol) were dissolved in NMP (4.4 mL). The resulting mixture was added to PS-DCC resin (156 mg, 1.33 mmol/g). The reaction was heated in the microwave to 100° C. for 420 sec. The crude mixture was purified using reverse phase HPLC. 1H NMR (500 MHz, DMSO-d₆) δ ppm 0.60-0.69 (m, 1 H), 0.95-1.04 (m, 1 H), 1.06-1.13 (m, 3 H), 1.20-1.26 (m, 1 H), 1.53-1.57 (m, 1 H), 4.16 (s, 2 H), 4.46 (s, 2 H), 6.89-6.98 (m, 1 H), 7.09-7.17 (m, 2 H), 7.26-7.35 (m, 6 H), 7.63-7.72 (m, 2 H), 8.26 (s, 1 H), 10.26 (s, 1 H), 11.68 (s, 1 H). MS (ESI) positive ion 404 (M+H); negative ion 402 (M−H).

### EXAMPLE 15

### N-(4-{2,4-diamino-6-[(benzyloxy)methyl]pyrimidin-5-yl}phenyl)-4-phenylbutanamide

The titled compound was synthesized according to the procedure described in Example 14, substituting 4-phenylbutyric acid for 2-methylcyclopropanecarboxylic acid. 1H NMR (500 MHz, DMSO-d₆) δ ppm 1.88-1.96 (m, 2 H), 2.31-2.39 (m, 2 H), 2.58-2.67 (m, 2 H), 4.17 (s, 2 H), 4.47 (s, 2 H), 6.90-6.98 (m, 1 H), 7.13-7.16 (m, 2 H), 7.18-7.24 (m, 3 H), 7.26-7.35 (m, 8 H), 7.66-7.74 (m, 2 H), 8.26 (s, 1 H), 10.05 (s, 1 H), 11.70 (s, 1 H). MS (ESI) positive ion 468 (M+H); negative ion 466 (M−H).

### EXAMPLE 16

### N-(4-{2,4-diamino-6-[(benzyloxy)methyl]pyrimidin-5-yl}-2-ethylphenyl)cyclopentanecarboxamide

### EXAMPLE 16A

### 5-(4-Amino-3-bromo-phenyl)-6-benzyloxymethyl-pyrimidine-2,4-diamine

To a solution of aniline from Example 6C (124 mg, 0.386 mmol) in methanol (3 mL) and acetic acid (glacial) (0.5 mL) was added bromine (20 mL, 0.386 mmol) dropwise at 0° C. The reaction mixture stirred at 0° C. for 30 minutes. It was concentrated under reduced pressure followed by addition of EtOAc (150 mL) and saturated NaHCO₃. The organic phase was separated and washed with brine, dried over MgSO4, filtered, concentrated and then purified by column chromatography to provide the titled compound (55 mg, 36%).

### EXAMPLE 16B

### 5-(4-Amino-3-ethyl-phenyl)-6-benzyloxymethyl-pyrimidine-2,4-diamine

To a mixture of Example 16A (20 mg, 0.05 mmol), (dppf)PdCl₂( 4 mg, 0.005 mmol), Cs₂CO₃ (98 mg, 0.3 mmol) in DMF was added 1M Et₃B in hexane (55 μL, 0.055 mmol). The reaction mixture was heated in a microwave oven at 100° C. for 10 minutes and then was filtered and purified by reverse phase HPLC (0-70% CH₃CN in aq. NH₄OAc) to provide the titled compound (7 mg, 41%).

### EXAMPLE 16C

### N-(4-{2,4-diamino-6-[(benzyloxy)methyl]pyrimidin-5-yl}-2-ethylphenyl)cyclopentanecarboxamide

The titled compound was synthesized according to the procedure described in Example 1, substituting aniline from Example 16B for aniline from Example 1C and cyclopentanecarboxylic acid for indan-2-carboxylic acid. ¹H NMR (300 MHz, DMSO-d₆) δ 9.16 (s, 1H), 7.53-6.40 (m, 12H), 4.40 (s, 2H), 4.08 (s, 2H), 2.93-2.88 (m, 1H), 2.60 (q, J=7.2 Hz, 2H), 1.90-1.52 (m, 8H), 1.09 (t, J=7.5 Hz, 3H). MS (ESI) positive ion 446 (M+H)⁺; negative ion 444 (M−H)⁻.

### EXAMPLE 17

### N-(4-{2,4-diamino-6-[(benzyloxy)methyl]pyrimidin-5-yl}phenyl)-2-(2,5-dichlorothien-3-yl)acetamide

The titled compound was prepared following the same procedure as described in Example 7, substituting (2,5-dichloro-thiophen-3-yl)-acetic acid for 4-chlorobenzoic acid used in Example 7. ¹H NMR (300 MHz, DMSO-d₆) δ 10.30 (s, 1H), 7.65 (d, J=8.4 Hz, 2H), 7.34-7.18 (m, 5H), 7.15 (d, J=8.4 Hz, 2H), 7.12 (s, 1H), 6.43 (s, 2H), 6.20 (s, 2H), 4.37 (s, 2H), 4.03 (s, 2H), 3.68 (s, 2H). MS (ESI) m/e 514, 516, 518 (M+H)⁺.

### EXAMPLE 18

### N-(4-{2,4-diamino-6-[(benzyloxy)methyl]pyrimidin-5-yl}phenyl)-2-phenylacetamide

The titled compound was synthesized according to the procedure described in Example 7, substituting phenylacetic acid for 4-chlorobenzoic acid. ¹H NMR (500 MHz, DMSO-d₆) δ 3.66 (s, 2 H), 3.95 (s, 2 H), 4.32 (s, 2 H), 5.60 (s, 2 H), 5.94 (s, 2 H), 7.14 (dd, J=12.16, 7.80 Hz, 4 H), 7.18-7.30 (m, 4 H), 7.30-7.39 (m, 4 H), 7.64 (d, J=8.42 Hz, 2 H), 10.21 (s, 1 H). MS (ESI) positive ion 440 (M+H)⁺; negative ion 438 (M−H)⁻.

### EXAMPLE 19

### N-(4-{2,4-diamino-6-[(benzyloxy)methyl]pyrimidin-5-yl}phenyl)-3,3-dimethylbutanamide

The titled compound was synthesized according to the procedure described in Example 14, substituting tert-butylacetic acid for 2-methylcyclopropanecarboxylic acid. 1H NMR (500 MHz, DMSO-d₆) δ ppm 1.04 (s, 10 H), 2.21 (s, 2 H), 4.17 (s, 2 H), 4.47 (s, 2 H), 6.95-7.00 (m, 1 H), 7.09-7.17 (m, 2 H), 7.26-7.34 (m, 5 H), 7.66-7.73 (m, 2 H), 8.25 (s, 1 H), 9.93 (s, 1 H), 11.62 (s, 1 H). MS (ESI) positive ion 420 (M+H); negative ion 418 (M−H).

### EXAMPLE 20

### N-(4-{2,4-diamino-6-[(benzyloxy)methyl]pyrimidin-5-yl}phenyl)-4-(4-methoxyphenyl)butanamide

The titled compound was prepared following the same procedure as described in Example 7, substituting 4-(4-methoxy-phenyl)-butyric acid for 4-chlorobenzoic acid used in Example 7. 1H NMR (300 MHz, DMSO-D6) δ ppm 1.85-1.92 (m, 2 H), 2.27-2.37 (m, 2 H), 2.57 (t, J=9 Hz, 2 H), 3.72 (s, 3H), 3.95 (s, 2 H), 4.33 (s, 2 H), 5.60 (bs, 2 H), 5.96 (s, 2 H), 6.86 (d, J=9 Hz, 2 H), 7.05-7.33 (m, 9 H), 7.63 (d, J=8.81 Hz, 2 H), 9.95 (s, 1 H). MS (ESI) m/e 498 (M+H)⁺.

### EXAMPLE 21

### N-(4-{2,4-diamino-6-[(benzyloxy)methyl]pyrimidin-5-yl}phenyl)cyclopentanecarboxamide

The titled compound was synthesized according to the procedure described in Example 14, substituting cyclopentanecarboxylic acid for 2-methylcyclopropanecarboxylic acid. 1H NMR (500 MHz, DMSO-d₆) δ ppm 1.48-1.96 (m, 8 H), 2.74-2.86 (m, 1 H), 4.14-4.23 (s, 2 H), 4.43-4.52 (s, 2 H), 6.66-6.72 (m, 1 H), 6.88-6.98 (m, 2 H), 7.11-7.18 (m, 2 H), 7.22-7.38 (m, 6 H), 7.67-7.76 (m, 2 H), 9.99-10.05 (s, 1 H), 11.65-11.74 (s, 1 H). MS (ESI) positive ion 418 (M+H); negative ion 416 (M−H).

### EXAMPLE 22

### N-(4-{2,4-diamino-6-[(benzyloxy)methyl]pyrimidin-5-yl}phenyl)cyclopropanecarboxamide

The titled compound was synthesized according to the procedure described in Example 14, substituting cyclopropanecarboxylic acid for 2-methylcyclopropanecarboxylic acid. 1H NMR (500 MHz, DMSO-d₆) δ ppm 0.67-0.92 (m, 4 H), 1.71-1.87 (m, 1 H), 4.16 (s, 2 H), 4.47 (s, 2 H), 6.86-7.02 (m, 2 H), 7.06-7.20 (m, 2 H), 7.19-7.41 (m, 5 H), 7.59-7.76 (m, 2 H), 8.10-8.37 (m, 1 H), 10.32 (s, 1 H), 11.67 (s, 1 H). MS (ESI) positive ion 390 (M+H); negative ion 388 (M−H).

### EXAMPLE 23

### N-(4-{2,4-diamino-6-[(benzyloxy)methyl}pyrimidin-5-yl}phenyl)-2-thien-3-ylacetamide

The titled compound was prepared following the same procedure as described in Example 7, substituting thiophen-3-yl-acetic acid for 4-chlorobenzoic acid used in Example 7. 1H NMR (300 MHz, DMSO-d₆) δ ppm 3.67 (s, 2 H), 3.95 (s, 2 H), 4.32 (s, 2 H), 5.61 (bs, 2 H), 5.95 (s, 2 H), 7.08-7.36 (m, 9 H), 7.49 (dd, J=4.92, 2.88 Hz, 1 H), 7.63 (d, J=8.48 Hz, 2 H), 10.19 (s, 1 H). MS (ESI) m/e 446 (M+H)⁺.

### EXAMPLE 24

### N-(4-{2,4-diamino-6-[(benzyloxy)methyl]pyrimidin-5-yl}phenyl)-3-phenylpropanamide

The titled compound was synthesized according to the procedure described in Example 14, substituting hydrocinnamic acid for 2-methylcyclopropanecarboxylic acid. 1H NMR (500 MHz, DMSO-d₆) δ ppm 2.60-2.76 (m, 2 H), 2.83-3.04 (m, 3 H), 4.17 (s, 2 H), 4.49 (s, 2 H), 6.88-7.05 (m, 1 H), 7.09-7.40 (m, 12 H), 7.61-7.80 (m, 2 H), 8.25 (s, 1 H), 10.08 (s, 1 H), 11.67 (s, 1 H). MS (ESI) positive ion 454 (M+H); negative ion 452 (M−H).

### EXAMPLE 25

### N-(4-{2,4-diamino-6-[(benzyloxy)methyl]pyrimidin-5-yl}phenyl)-2-methylcyclohexanecarboxamide

The titled compound was synthesized according to the procedure described in Example 14, substituting 2-methyl-1-cyclohexanecarboxylic acid for 2-methylcyclopropanecarboxylic acid. 1H NMR (500 MHz, DMSO-d₆) δ ppm 0.85-0.93 (m, 4 H), 1.27-1.33 (m, 1 H), 1.3-1.40 (m, 1 H), 1.47-1.55 (m, 4 H), 1.66-1.75 (m, 3 H), 4.18 (s, 2 H), 4.47 (s, 2 H), 6.89-6.98 (m, 1 H), 7.09-7.17 (m, 2 H), 7.26-7.36 (m, 6 H), 7.66-7.76 (m, 2 H), 8.25 (s, 1 H), 9.90 (s, 1 H), 11.68 (s, 1 H). MS (ESI) positive ion 446 (M+H); negative ion 444 (M−H).

### EXAMPLE 26

### N-(4-{2,4-diamino-6-[(benzyloxy)methyl]pyrimidin-5-yl}phenyl)-3-methylbutanamide

The titled compound was synthesized according to the procedure described in Example 14, substituting isovaleric acid for 2-methylcyclopropanecarboxylic acid. 1H NMR (500 MHz, DMSO-d₆) δ ppm 0.91-0.99 (m, 6 H), 2.05-2.14 (m, 1 H), 2.17-2.25 (m, 2 H), 4.18 (s, 2 H), 4.48 (s, 2 H), 6.94 (s, 1 H), 7.10-7.18 (m, 2 H), 7.26-7.35 (m, 6 H), 7.66-7.75 (m, 2 H), 8.26 (s, 1 H), 10.01 (s, 1 H), 11.69 (s, 1 H). MS (ESI) positive ion 406 (M+H); negative ion 404 (M−H)

### EXAMPLE 27

### N-(4-{2,4-diamino-6-[(benzyloxy)methyl]pyrimidin-5-yl}phenyl)-4-methylcyclohexanecarboxamide

The titled compound was synthesized according to the procedure described in Example 14, substituting 4-methyl-1-cyclohexanecarboxylic acid for 2-methylcyclopropanecarboxylic acid. 1H NMR (500 MHz, DMSO-d₆) δ ppm 0.86-0.95 (m, 4 H), 1.15-1.22 (m, 2 H), 1.40-1.48 (m, 2 H), 1.49-1.58 (m, 3 H), 1.72-1.81 (m, 3 H), 4.15 (s, 2 H), 4.44 (s, 2 H), 6.61-6.67 (m, 1 H), 6.84-6.93 (m, 1 H), 7.09-7.17 (m, 2 H), 7.24-7.33 (m, 7 H), 7.66-7.75 (m, 2 H). MS (ESI) positive ion 446 (M+H); negative ion 444 (M−H).

### EXAMPLE 28

### N-(4-{2,4-diamino-6-[(benzyloxy)methyl]pyrimidin-5-yl}phenyl)cycloheptanecarboxamide

The titled compound was synthesized according to the procedure described in Example 14, substituting cycloheptanecarboxylic acid for 2-methylcyclopropanecarboxylic acid. 1H NMR (500 MHz, DMSO-d₆) δ ppm 1.42-1.59 (m, 5 H), 1.59-1.69 (m, 1 H), 1.69-1.79 (m, 3 H), 1.78-1.94 (m, 3 H), 4.18 (s, 2 H), 4.48 (s, 2 H), 6.66-6.71 (m, 1 H), 6.88-6.98 (m, 1 H), 7.10-7.17 (m, 2 H), 7.25-7.37 (m, 6 H), 7.67-7.74 (m, 2 H), 8.25 (s, 1 H), 9.94 (s, 1 H), 11.66 (s, 1 H). MS (ESI) positive ion 446 (M+H); negative ion 444 (M−H).

### EXAMPLE 29

### 2-cyclopentyl-N-(4-{2,4-diamino-6-[(benzyloxy)methyl]pyrimidin-5-yl}phenyl)acetamide

The titled compound was synthesized according to the procedure described in Example 14, substituting cyclopentylacetic acid for 2-methylcyclopropanecarboxylic acid. 1H NMR (500 MHz, DMSO-d₆) δ ppm 1.15-1.24 (m, 2 H), 1.50-1.57 (m, 2 H), 1.58-1.64 (m, 2 H), 1.72-1.81 (m, 2 H), 2.22-2.28 (m, 1 H), 2.31-2.35 (m, 2 H), 4.18 (s, 2 H), 4.47 (s, 2 H), 6.90-6.98 (m, 1 H), 7.10-7.18 (m, 2 H), 7.26-7.35 (m, 6 H), 7.65-7.74 (m, 2 H), 8.26 (s, 1 H), 10.01 (s, 1H), 11.68 (s, 1 H). MS (ESI) positive ion 432 (M+H).

### EXAMPLE 30

### 2-(benzyloxy)-N-(4-{2,4-diamino-6-[(benzyloxy)methyl]pyrimidin-5-yl}phenyl)acetamide

The titled compound was synthesized according to the procedure described in Example 14, substituting benzyloxyacetic acid for 2-methylcyclopropanecarboxylic acid. 1H NMR (500 MHz, DMSO-d₆) δ ppm 4.16 (d, 4 H), 4.48 (s, 2 H), 4.61 (s, 2 H), 6.92-7.00 (m, 1 H), 7.13-7.22 (m, 2 H), 7.27-7.35 (m, 7 H), 7.37-7.42 (m, 4 H), 7.72-7.81 (m, 2 H), 8.26 (s, 1 H), 9.98 (s, 1 H), 11.69 (s, 1 H). MS (ESI) positive ion 470 (M+H); negative ion 468 (M−H).

### EXAMPLE 31

### N-(4-{2,4-diamino-6-[(benzyloxy)methyl]pyrimidin-5-yl}phenyl)cyclopent-3-ene-1-carboxamide

The titled compound was synthesized according to the procedure described in Example 7, substituting 3-cyclopentene carboxylic acid for 4-chlorobenzoic acid. ¹H NMR (300 MHz, DMSO-d₆) δ 2.55-2.64 (m, 4 H), 3.13-3.27 (m, 1 H), 3.98 (s, 2 H), 4.34 (s, 2 H) 5.60-5.66 (m, 2 H), 5.69 (s, 2 H), 6.07 (s, 2 H), 7.13 (d, J=8.48 Hz, 2 H), 7.16-7.33 (m, 5 H), 7.66 (d, J=8.48 Hz, 2 H), 10.00 (s, 1 H). MS (ESI) positive ion 416 (M+H)⁺; negative ion 414(M−H)⁻.

### EXAMPLE 32

### (2R)-N-(4-{2,4-diamino-6-[(benzyloxy)methyl]pyrimidin-5-yl}phenyl)-5-oxotetrahydrofuran-2-carboxamide

The titled compound was prepared as a white solid (75% yield) according to the procedure described in Example 1, substituting aniline from Example 6C for aniline from Example 1C and 5-oxo-tetrahydro-furan-2-carboxylic acid for indan-2-carboxylic acid used in Example 1D. ¹H NMR (300 MHz, DMSO-d₆) δ 1.86 (m, 1 H), 1.99 (m, 1 H), 2.56 (m, 1 H), 1.99 (m, 1 H), 3.96 (s, 2 H), 4.08 (m, 1 H), 4.33 (s, 2 H), 5.61 (s, 2 H), 5.96 (s, 2 H), 7.14 (d, J=9.0 Hz, 2 H), 7.18-7.31 (m, 5 H), 7.76 (d, J=9.0 Hz, 2 H) and 9.74 (s, 1 H). MS (ESI) positive ion 434 (M+H)⁺; negative ion 432 (M−H)⁻.

### EXAMPLE 33

### N-(4-{2,4-diamino-6-[(benzyloxy)methyl]pyrimidin-5-yl}phenyl)-2-[4-(methylsulfonyl)phenyl]acetamide

The titled compound was prepared as a white solid (70% yield) according to the procedure described in Example 1D, substituting aniline from Example 6C for aniline from Example 1C and (4-methanesulfonyl-phenyl)-acetic acid for indan-2-carboxylic acid used in Example 1D and HATU for TBTU used in Example 1D. ¹H NMR (300 MHz, DMSO-d₆) δ 3.20 (s, 3 H), 3.81 (s, 2 H), 3.97 (s, 2 H), 4.33 (s, 2 H), 5.81 (s, 2 H), 6.17 (s, 2 H), 7.14-7.90 (d, J=9.0Hz, 2 H), 7.18-7.29 (m, 5 H), 7.63 (q, J₁=6.0Hz, J₂=3.0Hz, 4 H), 7.90 (d, J=9.0 Hz, 2 H) and 10.33 (s, 1 H). MS (ESI) positive ion 518 (M+H)⁺; negative ion 516 (M−H)⁻.

### EXAMPLE 34

### N-(4-{2,4-diamino-6-[(benzyloxy)methyl]pyrimidin-5-yl}phenyl)-4-(3,4-dimethoxyphenyl)butanamide

The titled compound was prepared following the same procedure as described in Example 7, substituting 4-(3,4-dimethoxy-phenyl)-butyric acid for 4-chlorobenzoic acid used in Example 7. 1H NMR (500 MHz, DMSO-D6) δ ppm 2.02-2.08 (m, 2H), 2.49 (t, J=5 Hz, 2 H), 2.73 (t, J=5 Hz, 2 H), 3.87 (s, 3 H), 3.89 (s, 3 H), 4.12 (s, 2 H), 4.48 (s, 2 H), 5.76 (bs, 2 H), 6.11 (s, 2 H), 6.82-7.04 (m, 4 H), 7.24-7.47 (m, 6 H), 7.73-7.83 (m, 2 H), 10.10 (s, 1 H). MS (ESI) m/e 528 (M+H)⁺.

### EXAMPLE 35

### 2-cyclopropyl-N-(4-{2,4-diamino-6-[(benzyloxy)methyl]pyrimidin-5-yl}phenyl)acetamide

The titled compound was synthesized according to the procedure described in Example 14, substituting cyclopropylacetic acid for 2-methylcyclopropanecarboxylic acid. 1H NMR (500 MHz, DMSO-d₆) δ ppm 0.17-0.25 (m, 2 H), 0.46-0.54 (m, 2 H), 1.03-1.11 (m, 1 H), 2.19-2.26 (m, 2 H), 4.18 (s, 2 H), 4.47 (s, 2 H), 6.94 (s, 1 H), 7.11-7.18 (m, 2 H), 7.26-7.36 (m, 6 H), 7.67-7.75 (m, 2 H), 8.26 (s, 1 H), 9.97 (s, 1 H), 11.70 (s, 1 H). MS (ESI) positive ion 404 (M+H); negative ion 402(M−H).

### EXAMPLE 36

### N-(4-{2,4-diamino-6-[(benzyloxy)methyl]pyrimidin-5-yl}phenyl)thiophene-3-carboxamide

The titled compound was prepared following the same procedure as described in Example 7, substituting 3-thiophene carboxylic acid for 4-chlorobenzoic acid used in Example 7. 1H NMR (300 MHz, DMSO-d₆) δ ppm 3.98 (s, 2 H), 4.34 (s, 2 H), 5.64 (bs, 2 H), 5.97 (s, 2 H), 7.15-7.33 (m, 6 H), 7.66 (d, J=2.71 Hz, 2 H), 7.81 (d, J=8.48 Hz, 2 H), 8.33-8.39 (m, 1 H), 10.11 (s, 1 H); MS (ESI) m/e 432 (M+H)⁺.

### EXAMPLE 37

5-chloro-N-(4-{2,4-diamino-6-[(benzyloxy)methyl]pyrimidin-5-yl}phenyl)thiophene-3-carboxamide

### EXAMPLE 37A 5-Chloro-thiophene-3-carboxylic acid

To a suspension of N-chlorosuccinimide (0.8 g, 6 mmol) in CH₂Cl₂, was added a solution of thiophene-3-carboxylic acid (0.64 g, 5 mmol) in 1 mL CH₂Cl₂, followed by a few drops of HClO₄. After stirring at room temperature overnight, the solvent was removed in vacco. The crude reaction mixture was then purified via flash column chromatography using gradient eluent 5% to 15% to 100% ethyl acetate and hexane to give the titled compounds.

### EXAMPLE 37B

### 5-chloro-N-(4-{2,4-diamino-6-[(benzyloxy)methyl]pyrimidin-5-yl}phenyl)thiophene

The titled compound was prepared following the same procedure as described in Example 7, substituting 5-chloro-3-thiophene carboxylic acid for 4-chlorobenzoic acid used in Example 7. 1H NMR (300 MHz, DMSO-d₆) δ ppm 3.98 (s, 2 H), 4.34 (s, 2 H), 5.64 (bs, 2 H), 5.97 (s, 2 H), 7.15-7.30 (m, 7 H), 7.67 (d, J=1.70 Hz, 1 H), 7.79 (d, J=8.81 Hz, 2 H), 8.24 (d, J=2.03 Hz, 1 H), 10.13 (s, 1 H). MS (ESI) m/e 466 (M+H)⁺.

### EXAMPLE 38

### (2S)-N-(4-{2,4-diamino-6-[(benzyloxy)methyl]pyrimidin-5-yl}phenyl)-2-phenylbutanamide

The titled compound was synthesized according to the procedure described in Example 14, substituting (S)-2-phenyl-butyric acid for 2-methylcyclopropanecarboxylic acid. 1H NMR (500 MHz, DMSO-₆) δ ppm 10.25 (s, 1H), 7.71 (d, J=8.1 Hz, 2H), 7.45-7.20 (m, 10H), 7.13 (d, J=8.7 Hz, 2H), 4.45 (s, 2H), 4.16 (s, 2H), 3.60 (dd, J=8.7, 6.2 Hz, 1H), 2.16-2.01 (m, 2H), 0.89 (td, J=7.2, 2.2 Hz, 3H). MS (ESI) m/e 468 (M+H)⁺.

### EXAMPLE 39

### N-(4-{2,4-diamino-6-[(3-methylbutoxy)methyl]pyrimidin-5-yl}phenyl)cyclopentanecarboxamide

### EXAMPLE 39A

### 3-(Methylbutoxy)acetic acid

Allyl isoamyl glycolate (0.5 g, 2.68 mmol) was dissolved in MeOH (6 mL) and 2 M NaOH (6 mL) was added. After 1 hour, the mixture was concentrated under reduced pressure and the remainder acidified with 1 M HCl to pH 3, The solution was extracted with EtOAc (3×10 mL) and the combined organic layers washed with brine, dried over MgSO₄ filtered and concentrated to provide the title compound as a clear oil (371 mg, 95%).

### EXAMPLE 39B

### 3-(Methylbutoxy)acetyl chloride

3-(Methylbutoxy)acetic acid (1.6 g, 10.9 mmol) from Example 39A was dissolved in SOCl₂ (8 mL, 100 mmol) and heated to reflux for 3 hours. The mixture was cooled to room temperature and concentrated under reduced pressure. The resulting acid chloride was taken on to the next step without further purification.

### EXAMPLE 39C

### 4-(3-Methylbutoxy)-2-(4-nitrophenyl)-3-oxo-butyronitrile

To a solution of 4-nitrophenylacetonitrile (500 mg, 3.0 mmol) in CH₂Cl₂ (5 mL) at 0° C. was added Et₃N (0.86 mL, 6.0 mmol) and DMAP (38 mg, 0.3 mmol). A solution of 3 (methylbutoxy)acetyl chloride (10 mmol) from Example 39B in CH₂Cl₂ (2 mL) was slowly added. The reaction was warmed to room temperature and stirred for 1 hour. The mixture was diluted with EtOAc (20 mL) and washed with 1 M HCl (10 mL), brine (10 mL), dried over MgSO₄, filtered and concentrated to provide the titled compound as a dark green solid (580 mg, 66%).

### EXAMPLE 39D

### 6-(3-Methylbutoxymethyl)-5-(4-nitrophenyl)-pyrimidine-2,4-diamine

To a solution of 4-(3-methylbutoxy)-2-(4-nitrophenyl)-3-oxo-butyronitrile (580 mg, 2.0 mmol) from Example 39C in CH₂Cl₂ (4.5 mL) and MeOH (0.5 mL) at 0° C. was added trimethylsilyl-diazomethane (2.0 M in Et₂O, 3 mL, 6.0 mmol). The reaction was stirred at room temperature for 1 hour. Glacial acetic acid (3 mL) was slowly added to quench excess TMS-diazomethane. The mixture was diluted with EtOAc (20 mL) and washed with aqueous NaHCO₃ solution (2×10 mL), brine (10 mL), dried over MgSO₄, filtered and concentrated under reduced pressure. The residue was taken up in EtOH (10 mL) followed by the addition of guanidine HCl (190 mg, 2.0 mmol) in EtOH (2 mL) and KOEt (1.0 mL, 2.0 mmol). The mixture was heated to refluxed for 1 hour after which it was concentrated under reduced pressure, taken up in 2M NaOH (30 mL) and filtered. The resulting black solid was recrystallized from EtOH to provide the titled compound as a yellow solid (160 mg, 24%).

### EXAMPLE 39E

### 5-(4-Aminophenyl)-6-(3-methylbutoxymethyl)-pyrimidine-2,4-diamine

To a flask containing 6-(3-methylbutoxymethyl)-5-(4-nitrophenyl)-pyrimidine-2,4-diamine (150 mg, 0.453 mmol) from Example 39D was added 10% Pd/C (15 mg, 0.014 mmol) and glacial acetic acid (4.5 mL). The mixture was placed under an atmosphere of H₂ and stirred at room temperature for 4 hours. The mixture was filtered through Celite and concentrated under reduced pressure to provide the titled compound as a clear yellow oil (125 mg, 92%).

### EXAMPLE 39F

### N-(4-{2,4-diamino-6-[(3-methylbutoxy)methyl]pyrimidin-5-yl}phenyl)cyclopentanecarboxamide

The titled compound was synthesized according to the procedure described in Example 7, substituting cyclopentane carboxylic acid for 4-chlorobenzoic acid and 5-(4-aminophenyl)-6-(3-methylbutoxymethyl)-pyrimidine-2,4-diamine for the aniline from Example 6C. ¹H NMR (300 MHz, DMSO-d₆) δ 0.80 (d, J=6.78 Hz, 6 H), 1.26 (q, J=6.78 Hz, 2 H), 1.48-1.90 (m, 9 H), 2.71-2.87 (m, 1 H), 3.24 (t, J=6.78 Hz, 2 H), 3.87 (s, 2 H), 5.75 (s, 2 H), 6.06 (s, 2 H), 7.11 (d, J=8.48 Hz, 2 H), 7.66 (d, J=8.48 Hz, 2 H), 9.92 (s, 1 H). MS (ESI) positive ion 398 (M+H)⁺; negative ion 396 (M−H)⁻.

### EXAMPLE 40

### 2-(2-chlorothien-3-yl)-N-(4-{2,4-diamino-6-[(benzyloxy)methyl]pyrimidin-5-yl}phenyl)acetamide

The titled compound was prepared following the same procedure as described in Example 7, substituting (2-chloro-thiophen-3-yl)-acetic acid for 4-chlorobenzoic acid used in Example 7. 1H NMR (300 MHz, DMSO-d₆) δ ppm 10.29 (s, 2H), 7.66 (d, J=8.7 Hz, 2H), 7.4 (d, J=5.9 Hz, 1H), 7.33-7.17 (m, 5H), 7.15 (d, J=8.4 Hz, 2H), 7.06 (d, J=5.6 Hz, 1H), 6.44 (s, 2H), 6.24 (s, 2H), 4.38 (s, 2H), 4.03 (s, 2H), 3.69 (s, 2H). MS (ESI) m/e480, 482 (M+H)³⁰ .

### EXAMPLE 41

### N-(4-{2,4-diamino-6-[(benzyloxy)methyl]pyrimidin-5-yl}phenyl)-2,2,3,3-tetramethylcyclopropanecarboxamide

The titled compound was prepared as a white solid (40% yield) according to the procedure described in Example 1D, substituting aniline from Example 6C for aniline from Example 1C and 2,2,3,3-tetramethylcyclopropane carboxylic acid for indan-2-carboxylic acid used in Example 1D and HATU for TBTU used in Example 1D. ¹H NMR (300 MHz, DMSO-d₆) δ 1.20 (s, 6 H), 1.26 (s, 6 H), 3.97 (s, 2 H), 4.34 (s, 2 H), 5.68 (s, 2 H), 6.03 (s, 2 H), 7.10 (d, J=9.0 Hz, 2H), 7.17-7.32 (m, 5 H), 7.62 (d, J=9.0 Hz, 2 H) and 9.93 (s, 1 H). MS (ESI) positive ion 446 (M+H)⁺; negative ion 444 (M−H)⁻.

### EXAMPLE 42

### 2-(4-chlorophenyl)-N-(4-{2,4-diamino-6-[(benzyloxy)methyl]pyrimidin-5-yl}phenyl)acetamide

The titled compound was synthesized according to the procedure described in Example 1, substituting aniline from Example 6C for aniline from Example 1C and (4-chloro-phenyl)-acetic acid for indan-2-carboxylic acid. ¹H NMR (300 MHz, DMSO-d₆) δ 10.24 (s, 1H), 7.63 (d, J=8.8 Hz, 2H), 7.43-7.12 (m, 11H), 5.96 (s, 2H), 5.60 (bs, 2H), 4.32 (s, 2H), 3.95 (s, 2H), 3.67 (s, 2H). MS (ESI) positive ion 474 (M+H)⁺; negative ion 472 (M−H)⁻.

### EXAMPLE 43

### 2-(2-chlorophenyl)-N-(4-{2,4-diamino-6-[(benzyloxy)methyl]pyrimidin-5-yl}phenyl)acetamide

The titled compound was synthesized according to the procedure described in Example 1, substituting Example 6C for Example 1C and (2-chloro-phenyl)-acetic acid for indan-2-carboxylic acid. ¹H NMR (300 MHz, DMSO-d₆) δ 10.28 (s, 1H), 7.65 (d, J=8.5 Hz, 2H), 7.43-7.12 (m, 11H), 5.97 (s, 2H), 5.62 (bs, 2H), 4.33 (s, 2H), 3.96 (s, 2H), 3.86 (s, 2H). MS (ESI) positive ion 474 (M+H)⁺; negative ion 472 (M−H)⁻.

### EXAMPLE 44

### N-(4-{2,4-diamino-6-[(benzyloxy)methyl]pyrimidin-5-yl}phenyl)-2-(2-methylphenyl)acetamide

The titled compound was synthesized according to the procedure described in Example 1, substituting aniline from Example 6C for aniline from Example 1C and (2-methyl-phenyl)-acetic acid for indan-2-carboxylic acid. ¹H NMR (300 MHz, DMSO-d₆) δ 10.21 (s, 1H), 7.65 (d, J=8.5 Hz, 2H), 7.31-7.12 (m, 11H), 6.03 (s, 2H), 5.62 (bs, 2H), 4.33 (s, 2H), 3.97 (s, 2H), 3.70 (s, 2H), 2.31 (s, 3H). MS (ESI) positive ion 454 (M+H)⁺; negative ion 452 (M−H)⁻.

### EXAMPLE 45

### N-(4-{2,4-diamino-6-[(benzyloxy)methyl]pyrimidin-5-yl}phenyl)-2-thien-2-ylacetamide

The titled compound was synthesized according to the procedure described in Example 1, substituting Example 6C for Example 1C and thiophen-2-yl-acetic acid for indan-2-carboxylic acid. ¹H NMR (300 MHz, DMSO-d₆) δ 10.26 (s, 1H), 7.63 (d, J=8.5 Hz, 2H), 7.42-6.96 (m, 10H), 5.95 (s, 2H), 5.62 (bs, 2H), 4.32 (s, 2H), 3.95 (s, 2H), 3.89 (s, 2H). MS (ESI) positive ion 446 (M+H)⁺; negative ion 444 (M−H)⁻.

### EXAMPLE 46

### N-(4-{2,4-diamino-6-[(benzyloxy)methyl]pyrimidin-5-yl}phenyl)-4-oxo-4-phenylbutanamide

The titled compound was synthesized according to the procedure described in Example 7, substituting 4-oxo-4-phenyl-butyric acid for 4-chlorobenzoic acid. ¹H NMR (300 MHz, DMSO-d₆) δ 10.15 (s, 1H), 8.01 (d, J=6.8 Hz, 2H), 7.70-7.61 (m, 3H), 7.55 (t, J=7.5, 2H), 7.35-7.18 (m, 5H), 7.13 (d, J=8.8 Hz, 2H), 6.54 (bs, 4H), 4.39 (s, 2H), 4.05 (s, 2H), 3.37 (t, J=6.3 Hz, 2H), 2.75 (t, J=6.3 Hz, 2H). MS (ESI) m/e 482 (M+H)⁺.

### EXAMPLE 47

### isobutyl 4-{2,4-diamino-6-[(benzyloxy)methyl]pyrimidin-5-yl}phenylcarbamate

5-(4-Amino-phenyl)-6-benzyloxymethyl-pyrimidine-2,4-diamine (50 mg, 0.16 mmol) from Example 6C was added to DMF (0.7 mL). The mixture was cooled to 0° C. and isobutylchloroformate (22 mg, 0.16 mmol) was added dropwise and allowed to stir for 5 min. MeOH (1 mL) was added to the mixture and the reaction mixture was purified by RP-HPLC (5-100% CH₃CN in aqueous NH₄OAc). The titled compound (25 mg, 37%) was isolated as a white solid. ¹H NMR (300 MHz, DMSO-d₆) δ 0.95 (d, J=6.78 Hz, 6 H), 1.77-2.10 (m, 1 H), 3.89 (d, J=6.78 Hz, 2 H), 3.95 (s, 2 H), 4.33 (s, 2 H), 5.61 (s, 2 H), 5.96 (s, 2 H), 7.11 (d, J=8.48 Hz, 2 H), 7.14-7.33 (m, 5 H), 7.50 (d, J=8.48 Hz, 2 H), 9.68 (s, 1 H). MS (ESI) positive ion 422 (M+H)⁺; negative ion 420 (M−H)⁻.

### EXAMPLE 48

### N-(4-{2,4-diamino-6-[(benzyloxy)methyl]pyrimidin-5-yl}phenyl)-N′-isopropylurea

The titled compound was synthesized according to the procedure described in Example 6, substituting isopropylamine for α-methyl-4-chloro-benzylamine. ¹H NMR (300 MHz, DMSO-d₆) δ 8.35 (s, 1H), 7.41 (d, J=8.8 Hz, 2H), 7.32-7.16 (m, 5H), 7.06 (d, J=8.5 Hz, 2H), 6.00 (d, J=7.8 Hz, 1H), 5.93 (s, 2H), 5.58 (bs, 2H), 4.33 (s, 2H), 3.96 (s, 2H), 3.84-3.71 (m, 1H), 1.10 (d, J=6.5 Hz, 3H). MS (ESI) positive ion 407 (M+H)⁺; negative ion 405 (M−H)⁻.

### EXAMPLE 49

### N-(4-{2,4-diamino-6-[(benzyloxy)methyl]pyrimidin-5-yl}phenyl)-2-[4-(trifluoromethyl)phenyl]acetamide

The titled compound was synthesized according to the procedure described in Example 1, substituting aniline from Example 6C for aniline from Example 1C and (4-trifluoromethyl-phenyl)-acetic acid for indan-2-carboxylic acid. ¹H NMR (300 MHz, DMSO-d₆) δ 10.31 (s, 1H), 7.75-7.12 (m, 13H), 5.98 (s, 2H), 5.62 (bs, 2H), 4.32 (s, 2H), 3.95 (s, 2H), 3.79 (s, 2H). MS (ESI) positive ion 508 (M+H)⁺; negative ion 506 (M−H)³¹.

### EXAMPLE 50

### but-3-enyl 4-{2,4-diamino-6-[(benzyloxy)methyl]pyrimidin-5-yl}phenylcarbamate

The titled compound was synthesized according to the procedure described in Example 47, substituting allyl chloroformate for isobutylchloroformate used in Example 47. ¹H NMR (300 MHz, DMSO-d₆) δ 2.41 (q, J=6.78 Hz, 2 H), 3.95 (s, 2 H), 4.16 (t, J=6.61 Hz, 2 H), 4.33 (s, 2 H), 5.02-5.26 (m, 2 H), 5.60 (s, 2 H), 5.77-5.92 (m, 1 H), 5.95 (s, 2 H), 7.11 (d, J=8.48 Hz, 2 H), 7.14-7.33 (m, 5 H), 7.50 (d, J=8.82 Hz, 2 H), 9.70 (s, 1 H). MS (ESI) positive ion 420 (M+H)⁺; negative ion 418 (M−H)⁻.

### EXAMPLE 51

### N-(4-{2,4-diamino-6-[(benzyloxy)methyl]pyrimidin-5-yl}phenyl)-2,2-dimethylpentanamide

The titled compound was prepared as a white solid (35% yield) according to the procedure described in Example 1D, substituting aniline from Example 6C for aniline from Example 1C and 2,2,-dimethyl-pentanoic acid for indan-2-carboxylic acid used in Example 1D and HATU for TBTU used in Example 1D. ¹H NMR (300 MHz, DMSO-d₆) δ 0.87 (q, J=9.0 Hz, 3H), 1.07 (s, 3 H), 1.20 (s, 3 H), 1.42 (m, 2H), 1.60 (m, 2H), 3.96 (s, 2 H), 4.34 (s, 2 H), 5.62 (s, 2 H), 5.96 (s, 2 H), 7.12 (d, J=9.0 Hz, 2H), 7.14-7.31 (m, 5 H), 7.71 (d, J=9.0 Hz, 2 H) and 9.20 (s, 1 H). MS (ESI) positive ion 434 (M+H)⁺; negative ion 432 (M−H)⁻.

### EXAMPLE 52

### N-(4-{2,4-diamino-6-[(benzyloxy)methyl]pyrimidin-5-yl}phenyl)-2-(2-fluorophenyl)acetamide

The titled compound was synthesized according to the procedure described in Example 1, substituting Example 6C for Example 1C and (2-fluoro-phenyl)-acetic acid for indan-2-carboxylic acid. ¹H NMR (300 MHz, DMSO-d₆) δ 10.28 (s, 1H), 7.64 (d, J=8.5 Hz, 2H), 7.45-7.12 (m, 11H), 5.96 (s, 2H), 5.61 (bs, 2H), 4.32 (s, 2H), 3.95 (s, 2H), 3.75 (s, 2H). MS (ESI) positive ion 458 (M+H)⁺; negative ion 456 (M−H)⁻.

### EXAMPLE 53

### 2-(3-chlorophenyl)-N-(4-{2,4-diamino-6-[(benzyloxy)methyl]pyrimidin-5-yl}phenyl)acetamide

The titled compound was synthesized according to the procedure described in Example 1, substituting aniline from Example 6C for aniline from Example 1C and (3-chloro-phenyl)-acetic acid for indan-2-carboxylic acid. ¹H NMR (300 MHz, DMSO-d₆) δ 10.26 (s, 1H), 7.64 (d, J=8.5 Hz, 2H), 7.43-7.12 (m, 11H), 6.00 (s, 2H), 5.64 (bs, 2H), 4.32 (s, 2H), 3.95 (s, 2H) 3.69 (s, 2H). MS (ESI) positive ion 474 (M+H)⁺; negative ion 472 (M−H)⁻.

### EXAMPLE 54

### N-(tert-butyl)-N′-(4-{2,4-diamino-6-[(benzyloxy)methyl]pyrimidin-5-yl}phenyl)urea

The titled compound was synthesized according to the procedure described in Example 6, substituting t-butylamine for α-methyl-4-chloro-benzylamine. ¹H NMR (300 MHz, DMSO-d₆) δ 8.30 (s, 1H), 7.39 (d, J=8.5 Hz, 2H), 7.35-7.16 (m, 5H), 7.05 (d, J=8.5 Hz, 2H), 6.02 (s, 2H), 6.00 (s, 1H), 5.64 (bs, 2H), 4.35 (s, 2H), 3.98 (s, 2H), 1.30 (s, 9H). MS (ESI) positive ion 421 (M+H)⁺; negative ion 419 (M−H)⁻.

### EXAMPLE 55

### N-(4-{2,4-diamino-6-[(benzyloxy)methyl]pyrimidin-5-yl}phenyl)-1-hydroxycyclopropanecarboxamide

The titled compound was prepared as a white solid (38% yield) according to the procedure described in Example 1D, substituting aniline from Example 6C for aniline from Example 1C and 1-hydroxy-cyclopropanecarboxylic acid for indan-2-carboxylic acid used in Example 1D and HATU for TBTU used in Example 1D. ¹H NMR (300 MHz, DMSO-d₆) δ 0.98 (q, J=4.5 Hz, 2H), 1.17 (q, J=4.5 Hz, 2H), 3.96 (s, 2 H), 4.33 (s, 2 H), 5.61 (s, 2 H), 5.97 (s, 2 H), 6.57 (s, 1 H), 7.14 (d, J=9.0 Hz, 2H), 7.17-7.32 (m, 5 H), 7.80 (d, J=9.0 Hz, 2 H) and 9.83 (s, 1 H). MS (ESI) positive ion 406 (M+H)⁺; negative ion 404 (M−H)⁻.

### EXAMPLE 56

### N-(4-{2,4-diamino-6-[(benzyloxy)methyl]pyrimidin-5-yl}phenyl)-N′-phenylurea

The titled compound was synthesized according to the procedure described in Example 6, substituting aniline for α-methyl-4-chloro-benzylamine. ¹H NMR (300 MHz, DMSO-d₆) δ 3.97 (s, 2 H), 4.35 (s, 2 H), 5.60 (s, 2 H), 5.95 (s, 2 H), 6.97 (t, J=7.29 Hz, 1 H), 7.12 (d, J=8.48 Hz, 2 H), 7.17-7.22 (m, 2 H), 7.23-7.34 (m, 5 H), 7.48 (t, J=7.97 Hz, 4 H), 8.74 (s, 1 H), 8.80 (s, 1 H). MS (ESI) positive ion 441 (M+H)⁺; negative ion 439 (M−H)⁻.

### EXAMPLE 57

### N-(4-{2,4-diamino-6-[(benzyloxy)methyl]pyrimidin-5-yl}phenyl)indane-2-carboxamide

The titled compound was synthesized according to the procedure described in Example 1, substituting aniline from Example 6C for aniline from Example 1C. ¹H NMR (300 MHz, DMSO-d₆) δ 10.11 (s, 1H), 7.68 (d, J=8.8 Hz, 2H), 7.32-7.12 (m, 11H), 5.96 (s, 2H), 5.62 (bs, 2H), 4.33 (s, 2H), 3.96 (s, 2H), 3.48-3.36 (m, 1H), 3.20, 3.17 (s, s, 4H). MS(ESI) positive ion 466 (M+H)⁺; negative ion 464(M−H)⁻.

### EXAMPLE 58

### N-(4-{2,4-diamino-6-[(benzyloxy)methyl]pyrimidin-5-yl}phenyl)bicyclo[4.2.0]octa-1,3,5-triene-7-carboxamide

The titled compound was synthesized according to the procedure described in Example 1, substituting aniline from Example 6C for aniline from Example 1C and bicyclo[4.2.0]octa-1,3,5-triene-7-carboxylic acid for indan-2-carboxylic acid. ¹H NMR (300 MHz, DMSO-d₆) δ 10.28 (s, 1H), 7.69 (d, J=8.5 Hz, 2H), 7.31-7.12 (m, 11H), 5.96 (s, 2H), 5.62 (bs, 2H), 4.48-4.3 (m, 1H), 4.33 (s, 2H), 3.96 (s, 2H), 3.48-3.30 (m, 2H). MS (ESI) positive ion 452 (M+H)⁺; negative ion 450 (M−H)⁻.

### EXAMPLE 59

### N-cyclopentyl-N′-(4-{2,4-diamino-6-[(benzyloxy)methyl]pyrimidin-5-yl}phenyl)urea

The titled compound was synthesized according to the procedure described in Example 6, substituting cyclopentylamine for α-methyl-4-chloro-benzylamine. ¹H NMR (300 MHz, DMSO-d₆) δ 8.31 (s, 1H), 7.41 (d, J=8.5 Hz, 2H), 7.34-7.17 (m, 5H), 7.06 (d, J=8.5 Hz, 2H), 6.16 (d, J=7.1 Hz, 1H), 5.94 (s, 2H), 5.58 (bs, 2H), 4.33 (s, 2H), 3.98 (s, 2H), 4.00-3.88 (m, 1H), 1.91-1.35 (m, 8H). MS (ESI) positive ion 433 (M+H)⁺; negative ion 431 (M−H)⁻.

### EXAMPLE 60

### N-(sec-butyl)-N′-(4-{2,4-diamino-6-[(benzyloxy)methyl]pyrimidin-5-yl}phenyl)urea

The titled compound was synthesized according to the procedure described in Example 6, substituting sec-butylamine for α-methyl-4-chloro-benzylamine. ¹H NMR (300 MHz, DMSO-d₆) δ 8.35 (s, 1H), 7.42 (d, J=8.8 Hz, 2H), 7.35-7.17 (m, 5H), 7.05 (d, J=8.8 Hz, 2H), 5.97 (d, J=7.8 Hz, 1H), 5.96 (s, 2H), 5.60 (bs, 2H), 4.34 (s, 2H), 3.96 (s, 2H), 3.68-3.56 (m, 1H), 1.49-1.37 (m, 2H), 1.07 (d, J=6.8 Hz, 3H), 0.88 (t, J=7.5 Hz, 3H). MS (ESI) positive ion 421 (M+H)⁺negative ion 419 (M−H)⁻.

### EXAMPLE 61

### N-(4-{2,4-diamino-6-[(benzyloxy)methyl]pyrimidin-5-yl}phenyl)-N′-(1-methylbutyl)urea

The titled compound was synthesized according to the procedure described in Example 6, substituting 1-methyl-butylamine for α-methyl-4-chloro-benzylamine. ¹H NMR (500 MHz, DMSO-d₆) δ ppm 0.89 (t, 3H, J=7.2 Hz), 1.07 (d, 3H, J=6.6 Hz), 1.36 (m, 4 H), 3.7 (m, 1 H), 4.19 (s, 2 H), 4.48 (s, 2 H), 6.04 (d, 1 H, J=8.4 Hz), 6.91 (s, 1 H), 7.06 (d, 2 H, J=8.4 Hz), 7.31 (m, 6 H), 7.49 (d, 2 H, J=8.4 Hz), 8.25 (s, 1 H), 8.50 (s, 1 H), 11.64 (s, 1 H); MS (ESI) 435.1 (M+H).

### EXAMPLE 62

### N-(4-{2,4-diamino-6-[(benzyloxy)methyl]pyrimidin-5-yl}phenyl)-N′-(2-methylbutyl)urea

The titled compound was synthesized according to the procedure described in Example 6, substituting 2-methyl-butylamine for α-methyl-4-chloro-benzylamine. ¹H NMR (500 MHz, DMSO-d₆) δ ppm 0.86 (m, 6H), 1.13 (m, 1H,), 1.37 (m, 1 H), 1.48 (m, 1H), 2.95 (m, 1H), 3.04 (m, 1H), 4.19 (s, 2 H), 4.48 (s, 2 H), 6.24 (t, 1 H, J=6.0 Hz) 6.91 (s, 1 H) 7.06 (d, 2 H, J=8.4 Hz) 7.31 (m, 6 H) 7.49 (d, 2 H, J=8.4 Hz), 8.25 (s, 1 H), 8.61 (s, 1 H), 11.64 (s, 1 H); MS (ESI) 435.1 (M+H).

### EXAMPLE 63

### N-(4-{2,4-diamino-6-[(benzyloxy)methyl]pyrimidin-5-yl}phenyl)-N′-isobutylurea

The titled compound was synthesized according to the procedure described in Example 6, substituting isobutylamine for α-methyl-4-chloro-benzylamine. ¹H NMR (300 MHz, DMSO-d₆) δ 8.46 (s, 1H), 7.42 (d, J=8.8 Hz, 2H), 7.35-7.17 (m, 5H), 7.05 (d, J=8.8 Hz, 2H), 6.18 (t, J=6.0 Hz, 1H), 6.00 (s, 2H), 5.63 (bs, 2H), 4.34 (s, 2H), 3.97 (s, 2H), 2.94 (t, J=6.2 Hz, 2H), 1.78-1.62 (m, 1H), 0.88 (t, J=6.8 Hz, 6H). MS(ESI) positive ion 421 (M+H)⁺; negative ion 419 (M−H)⁻.

### EXAMPLE 64

### N-butyl-N′-(4-{2,4-diamino-6-[(benzyloxy)methyl]pyrimidin-5-yl}phenyl)urea

The titled compound was synthesized according to the procedure described in Example 6, substituting n-butylamine for α-methyl-4-chloro-benzylamine. ¹H NMR (500 MHz, DMSO-d₆) δ ppm 0.9 (t, 3H, J=7.2 Hz), 1.30 (m, 2 H), 1.42 (m, 2 H), 3.10 (m, 2 H), 4.18 (s, 2 H), 4.48 (s, 2 H), 6.22 (t, 1 H, J=5.7 Hz), 6.91 (s, 1 H), 7.06 (d, 2 H, J=8.8 Hz), 7.31 (m, 6 H), 7.49 (d, 2 H, J=8.8 Hz), 8.25 (s, 1 H), 8.62 (s, 1 H), 11.64 (s, 1 H); MS (ESI) 421.1 (M+H).

### EXAMPLE 65

### N-(4-{2,4-diamino-6-[(benzyloxy)methyl]pyrimidin-5-yl}phenyl)-N′-(1-ethylpropyl)urea

The titled compound was synthesized according to the procedure described in Example 6, substituting 1-ethylpropylamine for α-methyl-4-chloro-benzylamine. ¹H NMR (500 MHz, DMSO-d₆) δ ppm 0.86 (t, 6H, J=7.3 Hz), 1.35 (m, 2 H), 1.49 (m, 2 H), 3.50 (m, 1 H), 4.19 (s, 2 H), 4.48 (s, 2 H), 6.00 (d, 1 H, J=8.6 Hz), 6.90 (s, 1 H), 7.06 (d, 2 H, J=8.7 Hz) 7.31 (m, 6 H), 7.49 (d, 2 H, J=8.7 Hz), 8.25 (s, 1 H), 8.52 (s, 1 H), 11.61 (s, 1 H); MS (ESI) 435.4 (M+H)

### EXAMPLE 66

### N-(4-{2,4-diamino-6-[(benzyloxy)methyl]pyrimidin-5-yl}phenyl)-N′-neopentylurea

The titled compound was synthesized according to the procedure described in Example 6, substituting 2,2-dimethylpropylamine for α-methyl-4-chloro-benzylamine. ¹H NMR (500 MHz, DMSO-d₆) δ ppm 0.86 (s, 9H,), 2.93 (d, 2 H, J=6.2 Hz), 4.19 (s, 2 H), 4.48 (s, 2 H), 6.26 (t, 1 H, J=5.9 Hz), 6.90 (s, 1 H), 7.07 (d, 2 H, J=8.5 Hz), 7.31 (m, 6 H), 7.49 (d, 2 H, J=8.5 Hz), 8.25 (s, 1 H), 8.63 (s, 1 H), 11.63 (s, 1 H); MS (ESI) 435.4 (M+H)

### EXAMPLE 67

### N-(4-{2,4-diamino-6-[(benzyloxy)methyl]pyrimidin-5-yl}phenyl)-N′-(3-methylbutyl)urea

The titled compound was synthesized according to the procedure described in Example 6, substituting 3-methyl-butylamine for α-methyl-4-chloro-benzylamine. ¹H NMR (500 MHz, DMSO-d₆) δ ppm 0.89 (t, 6H, J=6.6 Hz), 1.34 (m, 2 H), 1.61 (m, 1 H), 3.12 (m, 2 H), 4.18 (s, 2 H), 4.48 (s, 2 H), 6.18 (t, 1 H, J=5.6 Hz), 6.90 (s, 1 H), 7.06 (d, 2 H, J=8.7 Hz), 7.31 (m, 6 H), 7.49 (d, 2 H, J=8.7 Hz), 8.25 (s, 1 H), 8.61 (s, 1 H), 11.63 (s, 1 H); MS (ESI) 435.1 (M+H)

### EXAMPLE 68

### N-(cyclohexylmethyl)-N′-(4-{2,4-diamino-6-[(benzyloxy)methyl]pyrimidin-5-yl}phenyl)urea

The titled compound was synthesized according to the procedure described in Example 6, substituting aminomethylcyclohexane for α-methyl-4-chloro-benzylamine. ¹H NMR (500 MHz, DMSO-d₆) δ ppm 0.92 (m, 2H), 1.16 (m, 3 H), 1.38 (m, 1 H), 1.67 (m, 5H), 2.95 (t, 2 H, J=6.2 Hz), 4.19 (s, 2 H), 4.48 (s, 2 H), 6.23 (t, 1 H, J=5.9 Hz), 6.91 (s, 1 H), 7.06 (d, 2 H, J=8.4 Hz), 7.30 (m, 6 H), 7.49 (d, 2 H, J=8.7 Hz), 8.25 (s, 1 H), 8.61 (s, 1 H), 11.63 (s, 1 H); MS (ESI) 461.1 (M+H).

### EXAMPLE 69

### N-benzyl-N′-(4-{2,4-diamino-6-[(benzyloxy)methyl]pyrimidin-5-yl}phenyl)urea

6-Benzyloxymethyl-5-(4-nitro-phenyl)-pyrimidine-2,4-diamine from Example 6C (14 mg, 0.04 mmol), was dissolved in THF (0.5 mL) followed by the addition of DIEA neat (11 mg, 0.09 mmol). A solution of triphosgene (4 mg, 0.01 mmol) in THF (0.5 mL) was then added slowly and a white precipitation was observed right away. The resulting mixture was shaken for 20 minutes at room temperature. Then benzylamine (9 mg, 0.09 mmol) dissolved in THF (0.4 mL) was added to above mixture and was shaken for another 30 minutes at room temperature. The crude mixture was purified using reverse phase HPLC. 1H NMR (500 MHz, DMSO-d₆) δ ppm 1.97-2.14 (m, 1 H), 4.14-4.24 (m, 3 H), 4.23-4.37 (m, 3 H), 4.44-4.53 (m, 3 H), 7.02-7.13 (m, 2 H), 7.23-7.41 (m, 12 H), 7.45-7.56 (m, 2 H). MS (ESI) positive ion 455 (M+H); negative ion 453 (M−H).

### EXAMPLE 70

### N-(allyloxy)-N′-(4-{2,4-diamino-6-[(benzyloxy)methyl]pyrimidin-5-yl}phenyl)urea

The titled compound was synthesized according to the procedure described in Example 6, substituting O-allyl-hydroxylamine for α-methyl-4-chloro-benzylamine. ¹H NMR (300 MHz, DMSO-d₆) δ ppm 9.48 (s, 1H), 8.84 (s, 1H), 7.63 (d, J=8.5 Hz, 2H), 7.11 (d, J=8.48 Hz, 2H), 7.34-7.15 (m, 5H), 6.13-5.99 (m, 1H), 5.97 (s, 2H), 5.62 (s, 2H), 5.36 (dd, J=17.3, 1.7 Hz, 1H), 5.27 (dd, J=10.3, 1.9 Hz, 1H), 4.34 (s, 2H), 431 (d, J=6.1 Hz, 2H), 3.97 (s, 2H). MS (ESI), m/e (M+H)⁺ 421.

### EXAMPLE 71

### N-(1-adamantylmethyl)-N′-(4-{2,4-diamino-6-[(benzyloxy)methyl]pyrimidin-5-yl}phenyl)urea

The titled compound was synthesized according to the procedure described in Example 6, substituting 1-adamantanemethylamine for α-methyl-4-chloro-benzylamine. ¹H NMR (500 MHz, DMSO-d₆) δ ppm 1.46 (m, 6H), 1.60 (m, 3 H), 1.69 (m, 3 H), 1.95 (m, 3H), 2.81 (d, 2 H, J=6.3 Hz), 4.19 (s, 2 H), 4.48 (s, 2 H), 6.20 (t, 1 H, J=6.2 Hz), 6.91 (s, 1 H), 7.06 (d, 2 H, J=8.4 Hz), 7.32 (m, 6 H), 7.49 (d, 2 H, J=8.4 Hz), 8.25 (s, 1 H), 8.61 (s, 1 H), 11.63 (s, 1 H); MS (ESI) 513.0 (M+H).

### EXAMPLE 72

### N-(4-{2,4-diamino-6-[(benzyloxy)methyl]pyrimidin-5-yl}phenyl)-N′-(3-methoxybenzyl)urea

The titled compound was synthesized according to the procedure described in Example 69, substituting 3-methoxybenzylamine for benzylamine used in Example 69. ¹H NMR (500 MHz, DMSO-d₆) δ ppm 2.04-2.07 (m, 1 H), 3.74-3.76 (m, 3 H), 4.16-4.22 (m, 3 H), 4.26-4.32 (m, 3 H), 4.47-4.51 (m, 3 H), 6.80-6.91 (m, 3 H), 7.07-7.12 (m, 2 H), 7.25-7.37 (m, 8 H), 7.48-7.53 (m, 2 H). MS (ESI) positive ion 485 (M+H); negative ion 483 (M−H).

### EXAMPLE 73

### N-(4-{2,4-diamino-6-[(benzyloxy)methyl]pyrimidin-5-yl}phenyl)-N′-(2-phenylethyl)urea

The titled compound was synthesized according to the procedure described in Example 69, substituting phenethylamine for benzylamine used in Example 69. ¹H NMR (500 MHz, DMSO-d₆) δ ppm 2.55-2.58 (m, 1 H), 2.76 (t, 2 H), 3.35 (t, 2 H), 3.65-3.71 (m, 1 H), 4.13-4.25 (m, 3 H), 4.44-4.53 (m, 3 H), 7.02-7.13 (m, 2 H), 7.20-7.37 (m, 12 H), 7.43-7.51 (m, 2 H). MS (ESI) positive ion 469 (M+H); negative ion 467 (M−H).

### EXAMPLE 74

### N-(4-{2,4-diamino-6-[(benzyloxy)methyl]pyrimidin-5-yl}phenyl)-N′-(1-phenylethyl)urea

The titled compound was synthesized according to the procedure described in Example 6, substituting α-methyl-benzylamine for α-methyl-4-chloro-benzylamine. ¹H NMR (300 MHz, DMSO-d₆) δ 8.45 (s, 1H), 7.42-7.16 (m, 12H), 7.05 (d, J=8.5 Hz, 2H), 6.62 (d, J=7.8 Hz, 1H), 5.93 (s, 2H), 5.58 (bs, 2H), 4.88-4.77 (m, 1H), 4.33 (s, 2H), 3.95 (s, 2H), 1.40 (d, J=7.1 Hz, 3H). MS (ESI) positive ion 469 (M+H)⁺; negative ion 467 (M−H)⁻.

### EXAMPLE 75

### N-(4-{2,4-diamino-6-[(benzyloxy)methyl]pyrimidin-5-yl}phenyl)-N′-[2-(methylthio)ethyl]urea

The titled compound was synthesized according to the procedure described in Example 6, substituting 2-methylsulfanyl-ethylamine for α-methyl-4-chloro-benzylamine. ¹H NMR (300 MHz, DMSO-d₆) δ 8.65 (s, 1H), 7.43 (d, J=8.5 Hz, 2H), 7.32-7.17 (m, 5H), 7.06 (d, J=8.5 Hz, 2H), 6.27 (t, J=6.0 Hz, 1H), 5.99 (s, 2H), 5.61 (bs, 2H), 4.34 (s, 2H), 3.97 (s, 2H), 3.35-3.28 (m, 2H), 2.58 (t, J=6.8 Hz, 2H), 2.09 (s, 3H). MS (ESI) positive ion 439 (M+H)⁺; negative ion 437 (M−H)⁻.

### EXAMPLE 76

### N-(4-{2,4-diamino-6-[(benzyloxy)methyl]pyrimidin-5-yl}phenyl)-N′-(2,2,2-trifluoroethyl)urea

The titled compound was synthesized according to the procedure described in Example 6, substituting 2,2,2-trifluoro-ethylamine for α-methyl-4-chloro-benzylamine. ¹H NMR (300 MHz, DMSO-d₆) δ 8.80 (s, 1H), 7.45 (d, J=8.4 Hz, 2H), 7.30-7.16 (m, 5H), 7.09 (d, J=8.4 Hz, 2H), 6.74 (t, J=6.6 Hz, 1H), 5.92 (s, 2H), 5.57 (bs, 2H), 4.34 (s, 2H), 3.96 (s, 2H), 3.98-3.90 (m, 2H). MS (ESI) positive ion 447 (M+H)⁺; negative ion 445 (M−H)⁻.

### EXAMPLE 77

### N-(cyclopropylmethyl)-N′-(4-{2,4-diamino-6-[(benzyloxy)methyl]pyrimidin-5-yl}phenyl)urea

The titled compound was synthesized according to the procedure described in Example 6, substituting cyclopropyl-methylamine for α-methyl-4-chloro-benzylamine. ¹H NMR (300 MHz, DMSO-d₆) δ 8.50 (s, 1H), 7.43 (d, J=8.5 Hz, 2H), 7.35-7.17 (m, 5H), 7.06 (d, J=8.5 Hz, 2H), 6.20 (t, J=6.0 Hz, 1H), 6.02 (s, 2H), 5.68 (bs, 2H), 4.35 (s, 2H), 3.98 (s, 2H), 2.98 (t, J=6.2 Hz, 2H), 1.00-0.16 (m, 5H). MS (ESI) positive ion 419 (M+H)⁺; negative ion 417 (M−H)⁻.

### EXAMPLE 78

### N-(4-{2,4-diamino-6-[(benzyloxy)methyl]pyrimidin-5-yl}phenyl)-N′-ethylurea

The titled compound was synthesized according to the procedure described in Example 6, substituting ethylamine HCl salt for α-methyl-4-chloro-benzylamine. ¹H NMR (300 MHz, DMSO-d₆) δ 1.06 (t, J=7.12 Hz, 3 H), 3.12 (dt, J=12.63, 7.25 Hz, 2 H), 3.96 (s, 2 H), 4.33 (s, 2 H), 5.56 (s, 2 H), 5.94 (s, 2 H), 6.09 (t, J=5.59 Hz, 1 H), 7.05 (d, J=8.48 Hz, 2 H), 7.14-7.34 (m, 5 H), 7.42 (d, J=8.48 Hz, 2 H), 8.47 (s, 1 H). MS (ESI) positive ion 393 (M+H)⁺; negative ion 391 (M−H)⁻.

### EXAMPLE 79

### N-[4-(2,4-diamino-6-{[(2,6-difluorobenzyl)oxy]methyl}pyrimidin-5-yl)phenyl]cyclopropanecarboxamide

### EXAMPLE 79A

### 5-(4-amino-phenyl)-6-bromomethyl-pyrimidine-2,4-diamine

5-(4-Amino-phenyl)-6-benzyloxymethyl-pyrimidine-2,4-diamine from Example 6C (642 mg, 2 mmol), H₂O (0.5 ml) and HBr in HOAc (9.5 ml, 33 wt %) were mixed in a sealed tube. The mixture was heated at 100° C. in a microwave for 2 h and cooled down to room temperature. The mixture was filtered and the solid was washed with acetonitrile (2 ml) to give a white salt of 5-(4-amino-phenyl)-6-bromomethyl-pyrimidine-2,4-diamine hydrogen bromine salt (656 mg, 72%). ¹H NMR (300 MHz, DMSO-d₆) δ 4.06 (s, 2H), 7.06 (d, J=9.0 Hz, 2H), 7.17 (d, J=9.0 Hz, 2H. MS (ESI) positive ion 295 (M+H).

### EXAMPLE 79B

### Cyclopropanecarboxylic acid [4-(2,4-diamino-6-bromomethyl-pyrimidin-5-yl)-phenyl]-amide

To a suspension of 5-(4-amino-phenyl)-6-bromomethyl-pyrimidine-2,4-diamine hydrogen bromine salt from Example 79A (454 mg, 1 mmol) in CH₂Cl₂ (10 ml) was added cyclopropanecarbonyl chloride (156.8 mg, 1.5 mmol), following diisopropylethylamine (580.5 mg, 4.5 mmol) dropwise at room temperature. The reaction mixture was stirred for 2 h at room temperature and then evaporated to remove off solvent. Water and ethyl acetate (30 ml, 1:1) were added, the organic layer was washed with water, dried over MgSO₄ and evaporated. The crude product was recrystalized from ethyl acetate and ether to give pure title compound (332 mg, 91%). ¹H NMR (300 MHz, DMSO-d₆) δ 0.80 (m, 4H), 1.80 (m, 1H), 3.93 (s, 2 H), 5.75 (s, 2 H), 6.05 (s, 2 H), 7.16 (d, J=9.0 Hz, 2H), 7.68 (d, J=9.0 Hz, 2 H) and 10.28 (s, 1 H). MS (ESI) positive ion 363.8 (M+H)⁺; negative ion 359.9 (M−H)⁻.

### EXAMPLE 79C

### N-[4-(2,4-diamino-6-{[(2,6-difluorobenzyl)oxy]methyl}pyrimidin-5-yl)phenyl]-cyclopropanecarboxamide

To 2,6-diflorobenzyl alcohol (72 mg, 0.5 mmol) in DMSO (5 ml) was added NaH (20 mg, 0.5 mmol, 60% in oil). The mixture was stirred at room temperature for 20 min and then cyclopropanecarboxylic acid [4-(2,4-diamino-6-bromomethyl-pyrimidin-5-yl)-phenyl]-amide from Example 79B (36 mg, 0.1 mmol) was added. The reaction mixture was stirred at room temperature for 2 h. LC/MS indicated the disappearance of starting material. Water and ethyl acetate (20 ml, 1:1) were added, the organic layer was washed with water and evaporated. The crude product was purified on reverse phase HPLC (NH₄OAc, 5-100%) to give the titled compound (28.5 mg, 67%). ¹H NMR (300 MHz, DMSO-d₆) δ 0.81 (m, 4H), 1.80 (m, 1H), 3.91 (s, 2H), 4.38 (s, 2H), 5.62 (s, 2H), 5.95 (s, 2 H), 7.04-7.10 (m, 3H), 7.42 (m, 2H), 7.60 (d, J=9.0 Hz, 2H) and 10.21 (s, 1H). MS (ESI) positive ion 426 (M+H)⁺; negative ion 424 (M−H)⁻.

### EXAMPLE 80

### N-[4-(2,4-diamino-6-{[(2,5-difluorobenzyl)oxy]methyl}pyrimidin-5-yl)phenyl]cyclopropanecarboxamide

The titled compound was made from cyclopropanecarboxylic acid [4-(2,4-diamino-6-bromomethyl-pyrimidin-5-yl)-phenyl]-amide from Example 79B and 2,5-diflorobenzyl alcohol as described in Example 79C. Yield 75%. ¹H NMR (300 MHz, DMSO-d₆) δ 0.81 (m, 4H), 1.81 (m, 1H), 4.00 (s, 2H), 4.39 (s, 2H), 5.62 (s, 2H), 5.96 (s, 2 H), 7.11 (d, J=9.0 Hz, 2H), 7.047.32 (m, 3H), 7.62 (d, J=9.0 Hz, 2H) and 10.22 (s, 1H). MS (ESI) positive ion 426 (M+H)⁺; negative ion 424 (M−H)⁻.

### EXAMPLE 81

### N-[4-(2,4-diamino-6-{[(2,3-difluorobenzyl)oxy]methyl}pyrimidin-5-yl)phenyl]cyclopropanecarboxamide

The titled compound was prepared from cyclopropanecarboxylic acid [4-(2,4-diamino-6-bromomethyl-pyrimidin-5-yl)-phenyl]-amide from Example 79B and 2,3-diflorobenzyl alcohol as described in Example 79C. Yield 70%. ¹H NMR (300 MHz, DMSO-d₆) δ 0.81 (m, 4H), 1.82 (m, 1H), 3.98 (s, 2H), 4.44 (s, 2H), 5.62 (s, 2H), 5.96 (s, 2 H), 7.11 (d, J=9.0 Hz, 2H), 7.08-7.35 (m, 3H), 7.61 (d, J=9.0 Hz, 2H), and 10.22 (s, 1H). MS (ESI) positive ion 426 (M+H)⁺; negative ion 424 (M−H)⁻.

### EXAMPLE 82

### N-[4-(2,4-diamino-6-{[(5-methyl-2-furyl)methoxy]methyl}pyrimidin-5-yl)phenyl]cyclopentanecarboxamide

The titled compound was synthesized according to the procedure described in Example 79, substituting (5-methyl-furan-2-yl)-methanol for (2,6-difluoro-phenyl)-methanol and cyclopentane carboxylic acid for cyclopropane carboxylic acid. ¹H NMR (300 MHz, DMSO-d₆) δ 9.92 (s, 1H), 7.65 (d, J=8.8 Hz, 2H), 7.10 (d, J=8.8 Hz, 2H), 6.16 (d, J=2.7 Hz, 1H), 6.01 (s, 2H), 5.97-5.93 (m, 1H), 5.64 (bs, 2H), 4.21 (s, 2H), 3.91 (s, 2H), 2.86-2.74 (m, 1H), 2.21 (s, 3H), 1.90-1.52 (m, 8H). MS (ESI) positive ion 422 (M+H)⁺; negative ion 420 (M−H)⁻.

### EXAMPLE 83

### N-[4-(2,4-diamino-6-{[(3-fluorobenzyl)oxy]methyl}pyrimidin-5-yl)phenyl]cyclopropanecarboxamide

The titled compound was prepared from cyclopropanecarboxylic acid [4-(2,4-diamino-6-bromomethyl-pyrimidin-5-yl)-phenyl]-amide of Example 79B and 3-florobenzyl alcohol as described in Example 79C. Yield 68%. ¹H NMR (300 MHz, DMSO-d₆) δ 0.81 (m, 4H), 1.81 (m, 1H), 3.98 (s, 2H), 4.36 (s, 2H), 5.64 (s, 2H), 5.98 (s, 2 H), 6.97-7.09 (m, 3H), 7.12 (d, J=9.0 Hz, 2H), 7.28-7.36 (m, 1H), 7.63 (d, J=9.0 Hz, 2H) and 10.24 (s, 1H). MS (ESI) positive ion 408 (M+H)⁺; negative ion 406 (M−H)⁻.

### EXAMPLE 84

### N-[4-(2,4-diamino-6-{[(3-methylbut-2-enyl)oxy]methyl}pyrimidin-5-yl)phenyl]cyclopropanecarboxamide

The titled compound was synthesized according to the procedure described in Example 79, substituting 3-methyl-but-2-en-1-ol for (2,6-difluoro-phenyl)-methanol. ¹H NMR (300 MHz, DMSO-d₆) δ 10.26 (s, 1H), 7.65 (d, J=8.5 Hz, 2H),7.12 (d, J=8.5 Hz, 2H), 6.22 (bs, 2H), 6.02 (bs, 2H), 5.17-5.09 (m, 1H), 3.90 (s, 2H), 3.79 (d, J=6.8 Hz, 2H), 1.94-1.84 (m, 1H), 1.64 (s, 3H), 1.53 (s, 3H), 0.82-0.78 (m, 4H). MS (ESI) positive ion 368 (M+H)⁺; negative ion 366 (M−H)⁻.

### EXAMPLE 85

### N-(4-{2,4-diamino-6-[(2-phenylethoxy)methyl]pyrimidin-5-yl}phenyl)cyclopropanecarboxamide

The titled compound was prepared from cyclopropanecarboxylic acid [4-(2,4-diamino-6-bromomethyl-pyrimidin-5-yl)-phenyl]-amide of Example 79B and phenethyl alcohol as described in Example 79C. Yield 72%. ¹H NMR (300 MHz, DMSO-d₆) δ 0.80 (m, 4H), 1.81 (m, 1H), 2.68 (t, J=6.0 Hz, 2H), 3.42 (d, J=6.0 Hz, 2H), 3.88 (s, 2H), 5.55 (s, 2H), 5.89 (s, 2 H), 7.13-7.26 (m, 5H), 7.59 (d, J=9.0 Hz, 2H) and 10.21 (s, 1H). MS (ESI) positive ion 404 (M+H)⁺; negative ion 402 (M−H)⁻.

1. A compound of formula (I), 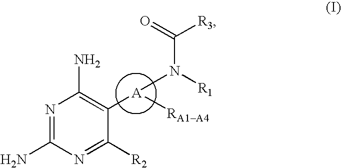
or a therapeutically suitable salt or prodrug thereof, wherein
A is a member selected from the group consisting of aryl, heteroaryl and heterocycle;
R₁ is a member selected from the group consisting of hydrogen, alkoxyalkyl, alkyl, aryl, arylalkyl, cycloalkyl, cycloalkylalkyl, haloalkyl, heteroaryl, heteroarylalkyl, heterocycle and heterocyclealkyl;
R₂ is a member selected from the group consisting of alkenyl, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylsulfinyl, alkylsulfinylalkyl, alkylsulfonyl, alkylsulfonylalkyl, alkylthio, alkylthioalkyl, alkynyl, alkynylalkoxyalkyl, aryl, arylalkenyl, arylalkenyloxyalkyl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, haloalkoxy, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxyalkyl, R_{C}R_{D}N—, (R_{C}R_{D}N)alkyl, (R_{C}R_{D}N)carbonylalkenyl, (R_{C}R_{D}N)carbonylalkyl, (R_{C}R_{D}N)sulfonyl and (R_{C}R_{D}N)sulfonylalkyl;
R₃ is a member selected from the group consisting of hydrogen, alkenyl, alkenyloxy, alkoxy, alkoxyalkoxy, alkoxyalkoxyalkyl, alkoxyalkyl, alkyl, alkylcarbonylalkyl, alkylsulfonylalkyl, alkylthioalkyl, alkynyl, aryl, arylalkoxy, arylalkoxyalkyl, arylalkyl, arylcarbonylalkyl, aryloxy, aryloxyalkyl, arylthioalkyl, cyanoalkyl, cycloalkenyl, cycloalkenylalkoxy, cycloalkenylalkoxyalkyl, cycloalkenylalkyl, cycloalkyl, cycloalkylalkoxy, cycloalkylalkoxyalkyl, cycloalkylalkyl, cycloalkylalkylthioalkyl, cycloalkyloxy, cycloalkyloxyalkyl, cycloalkylthioalkyl, haloalkoxy, haloalkyl, heteroaryl, heteroarylalkoxy, heteroarylalkoxyalkyl, heteroarylalkyl, heteroarylalkylthioalkyl, heteroaryloxy, heteroaryloxyalkyl, heteroarylthioalkyl, heterocycle, heterocyclealkoxy, heterocyclealkoxyalkyl, heterocyclealkyl, heterocyclealkylthioalkyl, heterocycleoxy, heterocycleoxyalkyl, heterocyclethioalkyl, hydroxyalkyl, R_{G}R_{H}N— and (R_{G}R_{H}N)alkyl;
R_{C} and R_{D} are each independently a member selected from the group consisting of hydrogen, alkoxyalkyl, alkoxyalkylcarbonyl, alkoxycarbonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (R_{E}R_{F}N)alkyl and (R_{E}R_{F}N)carbonyl, or R_{C} and R_{D} together with the nitrogen atom to which they are attached form a heterocycle;
R_{E} and R_{F} are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl;
R_{G} and R_{H} are each independently a member selected from the group consisting of hydrogen, alkenyl, alkenyloxy, alkoxyalkyl, alkoxyalkylcarbonyl, alkoxycarbonyl, alkoxysulfonyl, alkyl, alkylcarbonyl, alkyloxy, alkylsulfonyl, alkylthioalkyl, alkylthioalkylcarbonyl, alkylthiocarbonyl, aryl, arylalkoxy, arylalkoxyalkyl, arylalkyl, arylcarbonyl, cycloalkyl, cycloalkylalkoxyalkyl, cycloalkylalkyl, cycloalkylcarbonyl, cycloalkylalkoxy, formyl, haloalkyl, heteroaryl, heteroarylalkoxyalkyl, heteroarylalkyl, heteroarylcarbonyl, heteroarylalkoxy, heterocycle, heterocyclealkoxyalkyl, heterocyclealkyl, heterocyclecarbonyl, heterocyclealkoxy, (R_{J}R_{K}N)alkyl and (R_{J}R_{K}N)carbonyl, or R_{G} and R_{H} together with the nitrogen atom to which they are attached form a heterocycle;
R_{J} and R_{K} are each independently a member selected from the group consisting of hydrogen, alkoxycarbonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl and heterocyclecarbonyl;
R_{A1}, R_{A2}, R_{A3} and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, formyl, haloalkoxy, haloalkyl, halogen, heteroaryl, heterocycle, hydroxy, hydroxyalkyl, nitro, R_{M}R_{N}N—, (R_{M}R_{N}N)alkyl, (R_{M}R_{N}N)carbonyl and (R_{M}R_{N}N)sulfonyl; and
R_{M} and R_{N} are each independently a member selected from the group consisting of hydrogen, alkoxycarbonyl, alkyl, alkylcarbonyl, alkoxysulfonyl, alkylsulfonyl, aryl, arylalkyl and formyl, or R_{M} and R_{N} together with the nitrogen atom to which they are attached form a heterocycle.

2. The compound according to claim 1, wherein
A is phenyl.

3. The compound according to claim 1, wherein
A is phenyl;
R₁ is a member selected from the group consisting of hydrogen, alkoxyalkyl, alkyl, arylalkyl, cycloalkylalkyl, haloalkyl, heteroarylalkyl, heterocyclealkyl; and
R₂ is a member selected from the group consisting of alkenyl, alkenyloxyalkyl, alkoxyalkyl, alkoxycarbonyl, alkyl, alkylcarbonyl, aryl, arylalkoxyalkyl, arylalkyl, aryloxyalkyl, carboxy, carboxyalkyl, haloalkoxy, heteroaryl, heteroarylalkoxyalkyl, heteroarylalkyl, heteroaryloxyalkyl, heterocycle, heterocyclealkoxyalkyl, heterocyclealkyl, heterocycleoxyalkyl, hydroxyalkyl, R_{C}R_{D}N—, R_{C}R_{D}N)alkyl and (R_{C}R_{D}N)carbonylalkyl.

4. The compound according to claim 1, wherein
A is phenyl;
R₁ is a member selected from the group consisting of hydrogen, alkoxyalkyl, alkyl, arylalkyl, cycloalkylalkyl, haloalkyl, heteroarylalkyl, heterocyclealkyl;
R₂ is a member selected from the group consisting of alkenyl, alkenyloxyalkyl, alkoxyalkyl, alkoxycarbonyl, alkyl, alkylcarbonyl, aryl, arylalkoxyalkyl, arylalkyl, aryloxyalkyl, carboxy, carboxyalkyl, haloalkoxy, heteroaryl, heteroarylalkoxyalkyl, heteroarylalkyl, heteroaryloxyalkyl, heterocycle, heterocyclealkoxyalkyl, heterocyclealkyl, heterocycleoxyalkyl, hydroxyalkyl, —R_{C}R_{D}N, (R_{C}R_{D}N)alkyl and (R_{C}R_{D}N)carbonylalkyl; and
R₃ is a member selected from the group consisting of hydrogen, alkenyl, alkenyloxy, alkoxy, alkoxyalkoxyalkyl, alkoxyalkyl, alkyl, alkylcarbonylalkyl, aryl, arylalkoxyalkyl, arylalkyl, arylcarbonylalkyl, aryloxyalkyl, cycloalkenyl, cycloalkenylalkoxyalkyl, cycloalkyl, cycloalkylalkoxyalkyl, cycloalkylalkyl, cycloalkyloxyalkyl, haloalkyl, heteroaryl, heteroarylalkoxyalkyl, heteroarylalkyl, heteroaryloxyalkyl, heterocycle, heterocyclealkoxyalkyl, heterocyclealkyl, heterocycleoxyalkyl, hydroxy, hydroxyalkyl, nitro, R_{G}R_{H}N— and (R_{G}R_{H}N)alkyl.

5. The compound according to claim 1, wherein
A is phenyl;
R₁ is a member selected from the group consisting of hydrogen, alkoxyalkyl, alkyl, arylalkyl and haloalkyl;
R₂ is a member selected from the group consisting of alkenyloxyalkyl, alkoxyalkyl, alkoxycarbonyl, alkyl, alkylcarbonyl, arylalkoxyalkyl, arylalkyl, aryloxyalkyl, carboxy, carboxyalkyl, haloalkoxy, heteroarylalkoxyalkyl, heteroarylalkyl, heteroaryloxyalkyl, heterocyclealkoxyalkyl, heterocyclealkyl, heterocycleoxyalkyl, hydroxyalkyl, R_{C}R_{D}N—, (R_{C}R_{D}N)alkyl and (R_{C}R_{D}N)carbonylalkyl; and
R₃ is a member selected from the group consisting of hydrogen, alkenyl, alkenyloxy, alkoxy, alkoxyalkoxyalkyl, alkoxyalkyl, alkyl, alkylcarbonylalkyl, aryl, arylalkoxyalkyl, arylalkyl, arylcarbonylalkyl, aryloxyalkyl, cycloalkenyl, cycloalkenylalkoxyalkyl, cycloalkyl, cycloalkylalkoxyalkyl, cycloalkylalkyl, cycloalkyloxyalkyl, formyl, haloalkyl, heteroaryl, heteroarylalkoxyalkyl, heteroarylalkyl, heteroaryloxyalkyl, heterocycle, heterocyclealkoxyalkyl, heterocyclealkyl, heterocycleoxyalkyl, hydroxy, hydroxyalkyl, nitro, R_{G}R_{H}N— and (R_{G}R_{H}N)alkyl.

6. The compound according to claim 1, wherein
A is phenyl;
R₁ is a member selected from the group consisting of hydrogen and arylalkyl;
R₂ is a member selected from the group consisting of alkenyloxyalkyl, alkoxyalkyl, alkyl, arylalkoxyalkyl and heteroarylalkoxyalkyl;
R₃ is a member selected from the group consisting of hydrogen, alkenyloxy, alkoxy, alkoxyalkyl, alkyl, aryl, arylalkoxyalkyl, arylalkyl, arylcarbonylalkyl, cycloalkenyl, cycloalkyl, cycloalkylalkyl, formyl, heteroarylalkyl, heterocycle and R_{G}R_{H}N—; and
R_{G} and R_{H} are each independently a member selected from the group consisting of hydrogen, alkyl, alkylthioalkyl, aryl, arylalkyl, cycloalkyl, cycloalkylalkyl and haloalkyl.

7. The compound according to claim 1, that is a member selected from the group consisting of
N-[4-(2,4-diamino-6-ethylpyrimidin-5-yl)phenyl]indane-2-carboxamide;
N-[4-(2,4-diamino-6-ethylpyrimidin-5-yl)phenyl]-N′-(2-phenylethyl)urea;
N-(4-chlorobenzyl)-N′-[4-(2,4-diamino-6-ethylpyrimidin-5-yl)phenyl]urea;
N-[4-(2,4-diamino-6-ethylpyrimidin-5-yl)phenyl]-N′-( 1-phenylethyl)urea;
N-[1-(4-chlorophenyl)ethyl]-N′-[4-(2,4-diamino-6-ethylpyrimidin-5-yl)phenyl]urea;
N-[1-(4-chlorophenyl)ethyl]-N′-(4-{2,4-diamino-6-[(benzyloxy)methyl]pyrimidin-5-yl}phenyl)urea;
4-chloro-N-(4-{2,4-diamino-6-[(benzyloxy)methyl]pyrimidin-5-yl}phenyl)benzamide;
2-butoxy-N-(4-{2,4-diamino-6-[(benzyloxy)methyl]pyrimidin-5-yl}phenyl)acetamide;
N-(4-{2,4-diamino-6-[(benzyloxy)methyl]pyrimidin-5-yl}phenyl)propanamide;
N-(4-chlorobenzyl)-N-(4-{2,4-diamino-6-[(benzyloxy)methyl]pyrimidin-5-yl}phenyl)acetamide;
4-chlorobenzyl(4-{2,4-diamino-6-[(benzyloxy)methyl]pyrimidin-5-yl}phenyl)formamide;
N′-allyl-N-(4-chlorobenzyl)-N-(4-{2,4-diamino-6-[(benzyloxy)methyl]pyrimidin-5-yl}phenyl)urea;
2-(2-bromothien-3-yl)-N-(4-{2,4-diamino-6-[(benzyloxy)methyl]pyrimidin-5-yl}phenyl)acetamide;
N-(4-{2,4-diamino-6-[(benzyloxy)methyl]pyrimidin-5-yl}phenyl)-2-methylcyclopropanecarboxamide;
N-(4-{2,4-diamino-6-[(benzyloxy)methyl]pyrimidin-5-yl}phenyl)-4-phenylbutanamide;
N-(4-{2,4-diamino-6-[(benzyloxy)methyl]pyrimidin-5-yl}-2-ethylphenyl)cyclopentanecarboxamide;
N-(4-{2,4-diamino-6-[(benzyloxy)methyl]pyrimidin-5-yl}phenyl)-2-(2,5-dichlorothien-3-yl)acetamide;
N-(4-{2,4-diamino-6-[(benzyloxy)methyl]pyrimidin-5-yl}phenyl)-2-phenylacetamide;
N-(4-{2,4-diamino-6-[(benzyloxy)methyl]pyrimidin-5-yl}phenyl)-3,3-dimethylbutanamide;
N-(4-{2,4-diamino-6-[(benzyloxy)methyl]pyrimidin-5-yl}phenyl)-4-(4-methoxyphenyl)butanamide;
N-(4-{2,4-diamino-6-[(benzyloxy)methyl]pyrimidin-5-yl}phenyl)cyclopentanecarboxamide;
N-(4-{2,4-diamino-6-[(benzyloxy)methyl]pyrimidin-5-yl}phenyl)cyclopropanecarboxamide;
N-(4-{2,4-diamino-6-[(benzyloxy)methyl]pyrimidin-5-yl}phenyl)-2-thien-3-ylacetamide;
N-(4-{2,4-diamino-6-[(benzyloxy)methyl]pyrimidin-5-yl}phenyl)-3-phenylpropanamide;
N-(4-{2,4-diamino-6-[(benzyloxy)methyl]pyrimidin-5-yl}phenyl)-2-methylcyclohexanecarboxamide;
N-(4-{2,4-diamino-6-[(benzyloxy)methyl]pyrimidin-5-yl}phenyl)-3-methylbutanamide;
N-(4-{2,4-diamino-6-[(benzyloxy)methyl]pyrimidin-5-yl}phenyl)-4-methylcyclohexanecarboxamide;
N-(4-{2,4-diamino-6-[(benzyloxy)methyl]pyrimidin-5-yl}phenyl)cycloheptanecarboxamide;
2-cyclopentyl-N-(4-{2,4-diamino-6-[(benzyloxy)methyl]pyrimidin-5-yl}phenyl)acetamide;
2-(benzyloxy)-N-(4-{2,4-diamino-6-[(benzyloxy)methyl]pyrimidin-5-yl}phenyl)acetamide;
N-(4-{2,4-diamino-6-[(benzyloxy)methyl]pyrimidin-5-yl}phenyl)cyclopent-3-ene-1-carboxamide;
(2R)-N-(4-{2,4-diamino-6-[(benzyloxy)methyl]pyrimidin-5-yl}phenyl)-5-oxotetrahydrofuran-2-carboxamide;
N-(4-{2,4-diamino-6-[(benzyloxy)methyl]pyrimidin-5-yl}phenyl)-2-[4-(methylsulfonyl)phenyl]acetamide;
N-(4-{2,4-diamino-6-[(benzyloxy)methyl]pyrimidin-5-yl}phenyl)-4-(3,4-dimethoxyphenyl)butanamide;
2-cyclopropyl-N-(4-{2,4-diamino-6-[(benzyloxy)methyl]pyrimidin-5-yl}phenyl)acetamide;
N-(4-{2,4-diamino-6-[(benzyloxy)methyl]pyrimidin-5-yl}phenyl)thiophene-3-carboxamide;
5-chloro-N-(4-{2,4-diamino-6-[(benzyloxy)methyl]pyrimidin-5-yl}phenyl)thiophene-3-carboxamide;
(2S)-N-(4-{2,4-diamino-6-[(benzyloxy)methyl]pyrimidin-5-yl}phenyl)-2-phenylbutanamide;
N-(4-{2,4-diamino-6-[(3-methylbutoxy)methyl]pyrimidin-5-yl}phenyl)cyclopentanecarboxamide;
2-(2-chlorothien-3-yl)-N-(4-{2,4-diamino-6-[(benzyloxy)methyl]pyrimidin-5-yl}phenyl)acetamide;
N-(4-{2,4-diamino-6-[(benzyloxy)methyl]pyrimidin-5-yl}phenyl)-2,2,3,3-tetramethylcyclopropanecarboxamide;
2-(4-chlorophenyl)-N-(4-{2,4-diamino-6-[(benzyloxy)methyl]pyrimidin-5-yl}phenyl)acetamide;
2-(2-chlorophenyl)-N-(4-{2,4-diamino-6-[(benzyloxy)methyl]pyrimidin-5-yl}phenyl)acetamide;
N-(4-{2,4-diamino-6-[(benzyloxy)methyl]pyrimidin-5-yl}phenyl)-2-(2-methylphenyl)acetamide;
N-(4-{2,4-diamino-6-[(benzyloxy)methyl]pyrimidin-5-yl}phenyl)-2-thien-2-ylacetamide;
N-(4-{2,4-diamino-6-[(benzyloxy)methyl]pyrimidin-5-yl}phenyl)-4-oxo-4-phenylbutanamide;
isobutyl 4-{2,4-diamino-6-[(benzyloxy)methyl]pyrimidin-5-yl}phenylcarbamate;
N-(4-{2,4-diamino-6-[(benzyloxy)methyl]pyrimidin-5-yl}phenyl)-N′-isopropylurea;
N-(4-{2,4-diamino-6-[(benzyloxy)methyl]pyrimidin-5-yl}phenyl)-2-[4-(trifluoromethyl)phenyl]acetamide;
but-3-enyl 4-{2,4-diamino-6-[(benzyloxy)methyl]pyrimidin-5-yl}phenylcarbamate;
N-(4-{2,4-diamino-6-[(benzyloxy)methyl]pyrimidin-5-yl}phenyl)-2,2-dimethylpentanamide;
N-(4-{2,4-diamino-6-[(benzyloxy)methyl]pyrimidin-5-yl}phenyl)-2-(2-fluorophenyl)acetamide;
2-(3-chlorophenyl)-N-(4-{2,4-diamino-6-[(benzyloxy)methyl]pyrimidin-5-yl}phenyl)acetamide;
N-(tert-butyl)-N′-(4-{2,4-diamino-6-[(benzyloxy)methyl]pyrimidin-5-yl}phenyl)urea;
N-(4-{2,4-diamino-6-[(benzyloxy)methyl]pyrimidin-5-yl}phenyl)-1-hydroxycyclopropanecarboxamide;
N-(4-{2,4-diamino-6-[(benzyloxy)methyl]pyrimidin-5-yl}phenyl)-N′-phenylurea;
N-(4-{2,4-diamino-6-[(benzyloxy)methyl]pyrimidin-5-yl}phenyl)indane-2-carboxamide;
N-(4-{2,4-diamino-6-[(benzyloxy)methyl]pyrimidin-5-yl}phenyl)bicyclo[4.2.0]octa-1,3,5-triene-7-carboxamide;
N-cyclopentyl-N′-(4-{2,4-diamino-6-[(benzyloxy)methyl]pyrimidin-5-yl}phenyl)urea;
N-(sec-butyl)-N′-(4-{2,4-diamino-6-[(benzyloxy)methyl]pyrimidin-5-yl}phenyl)urea;
N-(4-{2,4-diamino-6-[(benzyloxy)methyl]pyrimidin-5-yl}phenyl)-N′-(1-methylbutyl)urea;
N-(4-{2,4-diamino-6-[(benzyloxy)methyl]pyrimidin-5-yl}phenyl)-N′-(2-methylbutyl)urea;
N-(4-{2,4-diamino-6-[(benzyloxy)methyl]pyrimidin-5-yl}phenyl)-N′-isobutylurea;
N-butyl-N′-(4-{2,4-diamino-6-[(benzyloxy)methyl]pyrimidin-5-yl}phenyl)urea;
N-(4-{2,4-diamino-6-[(benzyloxy)methyl]pyrimidin-5-yl}phenyl)-N′-(1-ethylpropyl)urea;
N-(4-{2,4-diamino-6-[(benzyloxy)methyl]pyrimidin-5-yl}phenyl)-N′-neopentylurea;
N-(4-{2,4-diamino-6-[(benzyloxy)methyl]pyrimidin-5-yl}phenyl)-N′-(3-methylbutyl)urea;
N-(cyclohexylmethyl)-N′-(4-{2,4-diamino-6-[(benzyloxy)methyl]pyrimidin-5-yl}phenyl)urea;
N-benzyl-N′-(4-{2,4-diamino-6-[(benzyloxy)methyl]pyrimidin-5-yl}phenyl)urea;
N-(allyloxy)-N′-(4-{2,4-diamino-6-[(benzyloxy)methyl]pyrimidin-5-yl}phenyl)urea;
N-(1-adamantylmethyl)-N′-(4-{2,4-diamino-6-[(benzyloxy)methyl]pyrimidin-5-yl}phenyl)urea;
N-(4-{2,4-diamino-6-[(benzyloxy)methyl]pyrimidin-5-yl}phenyl)-N′-(3-methoxybenzyl)urea;
N-(4-{2,4-diamino-6-[(benzyloxy)methyl]pyrimidin-5-yl}phenyl)-N′-(2-phenylethyl)urea;
N-(4-{2,4-diamino-6-[(benzyloxy)methyl]pyrimidin-5-yl}phenyl)-N′-(1-phenylethyl)urea;
N-(4-{2,4-diamino-6-[(benzyloxy)methyl]pyrimidin-5-yl}phenyl)-N′-[2-(methylthio)ethyl]urea;
N-(4-{2,4-diamino-6-[(benzyloxy)methyl]pyrimidin-5-yl}phenyl)-N′-(2,2,2-trifluoroethyl)urea;
N-(cyclopropylmethyl)-N′-(4-{2,4-diamino-6-[(benzyloxy)methyl]pyrimidin-5-yl}phenyl)urea;
N-(4-{2,4-diamino-6-[(benzyloxy)methyl]pyrimidin-5-yl}phenyl)-N′-ethylurea;
N-[4-(2,4-diamino-6-{[(2,6-difluorobenzyl)oxy]methyl}pyrimidin-5-yl)phenyl]cyclopropanecarboxamide;
N-[4-(2,4-diamino-6-{[(2,5-difluorobenzyl)oxy]methyl}pyrimidin-5-yl)phenyl]cyclopropanecarboxamide;
N-[4-(2,4-diamino-6-{[(2,3-difluorobenzyl)oxy]methyl}pyrimidin-5-yl)phenyl]cyclopropanecarboxamide;
N-[4-(2,4-diamino-6-{[(5-methyl-2-furyl)methoxy]methyl}pyrimidin-5-yl)phenyl]cyclopentanecarboxamide;
N-[4-(2,4-diamino-6-{[(3-fluorobenzyl)oxy]methyl}pyrimidin-5-yl)phenyl]cyclopropanecarboxamide;
N-[4-(2,4-diamino-6-{[(3-methylbut-2-enyl)oxy]methyl}pyrimidin-5-yl)phenyl]cyclopropanecarboxamide; and
N-(4-{2,4-diamino-6-[(2-phenylethoxy)methyl]pyrimidin-5-yl}phenyl)cyclopropanecarboxamide.

8. A pharmaceutical composition comprising a therapeutically effective amount of a compound of formula (I) and a pharmaceutically suitable carrier.

9. A method of treating a disorder regulated by GHS-Rs in a mammal, comprising administring of a compound of formula (I).

10. A method of treating disorders regulated by the activation of GHS-R, including Prader-Willi syndrome, eating disorder, weight gain, weight-loss maintainance following diet and exercise, obesity and disorders associated with obesity such as non-insulin dependent diabetes mellitus in a mammal comprising administrating a compound of formula (I).
